SEQUENCE LISTING (22 sequences)

SEQ ID NO: 1 - protein, 7 aa, Artificial Sequence
  Synthetic Peptide
GGGPGKR

SEQ ID NO: 2 - protein, 7 aa, Artificial Sequence
  Synthetic Peptide
RRGPGGG

SEQ ID NO: 3 - protein, 7 aa, Artificial Sequence
  Synthetic Peptide
GGGGGKR

SEQ ID NO: 4 - protein, 7 aa, Artificial Sequence
  Synthetic Peptide
RRGGGGG

SEQ ID NO: 5 - protein, 9 aa, Artificial Sequence
  Synthetic Peptide
GGAPGDVKR

SEQ ID NO: 6 - protein, 9 aa, Artificial Sequence
  Synthetic Peptide
RRAPGDVGG

SEQ ID NO: 7 - protein, 9 aa, Artificial Sequence
  Synthetic Peptide
GGYPGDVKR

SEQ ID NO: 8 - protein, 9 aa, Artificial Sequence
  Synthetic Peptide
RRYPGDVGG

SEQ ID NO: 9 - protein, 9 aa, Artificial Sequence
  Synthetic Peptide
GGHPGDVKR

SEQ ID NO: 10 - protein, 9 aa, Artificial Sequence
  Synthetic Peptide
RRHPGDVGG

SEQ ID NO: 11 - protein, 12 aa, Artificial Sequence
  Synthetic Peptide
QPLALEGSLQKR

SEQ ID NO: 12 - protein, 12 aa, Artificial Sequence
  Synthetic Peptide
GYGSSSRRAPQT

SEQ ID NO: 13 - protein, 11 aa, Artificial Sequence
  Synthetic Peptide
GYGSSSRRAPQ

SEQ ID NO: 14 - protein, 12 aa, Artificial Sequence
  Synthetic Peptide
GYGSSSAAAPQT

SEQ ID NO: 15 - protein, 7 aa, Artificial Sequence
  Synthetic Peptide
VGSSSGX

SEQ ID NO: 16 - protein, 7 aa, Artificial Sequence
  Synthetic Peptide
VGSSSXK

SEQ ID NO: 17 - protein, 7 aa, Artificial Sequence
  Synthetic Peptide
VGSSXGK

SEQ ID NO: 18 - protein, 7 aa, Artificial Sequence
  Synthetic Peptide
VGSXSGK

SEQ ID NO: 19 - protein, 7 aa, Artificial Sequence
  Synthetic Peptide
VGXSSGK

SEQ ID NO: 20 - protein, 7 aa, Artificial Sequence
  Synthetic Peptide
VXSSSGK

SEQ ID NO: 21 - protein, 7 aa, Artificial Sequence
  Synthetic Peptide
XGSSSGK

SEQ ID NO: 22 - protein, 44 aa, Artificial Sequence
  Synthetic construct
HGEGTFTSDLSKQMEEEAVRLFIEWLKNGGPSSGAPPSKKKKKK

(19) United States
(12) Patent Application Publication
(10) Pub. No.: US 2008/0280814 A1
(43) Pub. Date: Nov. 13, 2008

(54) Pharmaceutical Compositions Containing Insulin and Insulinotropic Peptide

(75) Inventors: Svend Ludvigsen, Lynge (DK); Morten Schlein, Kobenhavn S (DK)

Correspondence Address: NOVO NORDISK, INC.;INTELLECTUAL PROPERTY DEPARTMENT, 100 COLLEGE ROAD WEST, PRINCETON, NJ, 08540, US

(73) Assignee: Novo Nordisk A/S, Bagsvaerd (DK)

(21) Appl. No.: 11/663,111

(22) PCT Filed: Sep. 19, 2005
(86) PCT No.: PCT/DK05/00589

§ 371 Date: Jan. 24, 2008

(30) Foreign Application Priority Data
Sep. 17, 2004 (DK) PA 2004 01414

(51) Int. Cl. A61K 38/28; A61P 3/10

(52) U.S. Cl. 514/4

(57) ABSTRACT

Pharmaceutical composition for parenteral administration comprising insulin peptide an insulinotropic peptide and a ligand for the His^{b10} anion site.

### FIELD OF THE INVENTION

The present invention relates to the field of pharmaceutical compositions. More specifically the invention pertains to pharmaceutical compositions comprising two different pharmaceutically active peptides.

### BACKGROUND OF THE INVENTION

Diabetes mellitus is a metabolic disorder in which the ability to utilize glucose is partly or completely lost. About 5% of all people suffer from diabetes and the disorder approaches epidemic proportions. Since the introduction of insulin in the 1920's, continuous efforts have been made to improve the treatment of diabetes mellitus. Since people suffering from diabetes are subject to chronic treatment over several decades, there is a major need for safe, convenient and life quality improving insulin formulations.

In the treatment of diabetes mellitus, many varieties of insulin formulations have been suggested and used, such as regular insulin, isophane insulin (designated NPH), insulin zinc suspensions (such as Semilente®, Lente®, and Ultralente®), and biphasic isophane insulin.

Some of the commercial available insulin formulations are characterized by a fast onset of action and other formulations have a relatively slow onset but show a more or less prolonged action. Fast-acting insulin formulations are usually solutions of insulin, while retarded acting insulin formulations can be suspensions containing insulin in crystalline and/or amorphous form precipitated by addition of zinc salts alone or by addition of protamine or by a combination of both, or they may be soluble but precipitate upon injection.

Normally, insulin formulations are administered by subcutaneous injection. What is important for the patient is the action profile of the insulin formulation which is the action of insulin on the glucose metabolism as a function of the time from the injection. In this profile various parameters are important, e.g. the time for the onset, the maximum value, and the total duration of action. A variety of insulin formulations with different action profiles are desired and requested by the patients.

Human insulin consists of two polypeptide chains, the so-called A and B chains which contain 21 and 30 amino acid residues, respectively. The A and B chains are interconnected by two cysteine disulphide bridges. Insulin from most other species has a similar construction, but may not contain the same amino acid residues at the same positions. Within the last decade a number of human insulin analogues have been developed. They are designed for particular profiles of action, i.e. fast acting or prolonged action.

Insulin may be present in hexamer form. The insulin hexamer is an allosteric protein that exhibits both positive and negative cooperativity and half-of-the-sites reactivity in ligand binding. This allosteric behaviour consists of two interrelated allosteric transitions designated L^{A}₀ and L^{B}₀, three inter-converting allosteric conformation states (eq. 1),

$${T}_{6} {↔}^{ {L}_{0}^{A} } {T}_{3} {R}_{3} {↔}^{ {L}_{0}^{B} } {R}_{6} \qquad ( 1 )$$

designated T₆, T₃R₃, and R₆ and two classes of allosteric ligand binding sites designated as the phenolic pockets and the His^{B10} anion sites. These allosteric sites are associated only with insulin subunits in the R conformation. It has recently been found that presence of extended ligands for the His^{B10} anion sites may be utilised to obtain soluble insulin preparations with a prolonged action profile (WO 03/27081). Stabilisers for insulin preparations have been shown in WO 2004/056347.

Another peptide expected to become very important in the treatment of diabetes is glucagon-like peptide-1 (GLP-1). Human GLP-1 is a 37 amino acid residue peptide originating from preproglucagon which is synthesized i.a. in the L-cells in the distal ileum, in the pancreas and in the brain. GLP-1 is an important gut hormone with regulatory function in glucose metabolism and gastrointestinal secretion and metabolism. GLP-1 stimulates insulin secretion in a glucose-dependant manner, stimulates insulin biosynthesis, promotes beta cell rescue, decreases glucagon secretion, gastric emptying and food intake. A simple system is used to describe fragments and analogues of this peptide. Thus, for example, Gly⁸-GLP-1(7-37) designates an analogue of GLP-1(7-37) formally derived from GLP-1(7-37) by substituting the naturally occurring amino acid residue in position 8 (Ala) by Gly. Similarly, Lys³⁴(N^{ε}-tetradecanoyl)-GLP-1(7-37) designates GLP-1(7-37) wherein the 6-amino group of the Lys residue in position 34 has been tetradecanoylated. PCT publications WO 98/08871 and WO 99/43706 disclose stable derivatives of GLP-1 analogues, which have a lipophilic substituent. These stable derivatives of GLP-1 analogues have a protracted profile of action compared to the corresponding GLP-1 analogues.

A combination formulation comprising an insulin peptide and a GLP-1 peptide, may with a fixed ratio of the two pharmaceuticals, be a very efficacious treatment as well as one requiring less injections when administered to the same patient. However, such mixtures of the two peptides may present problems in terms of insufficient stability of the combined preparation. Thus, there is a big need for stable pharmaceutical compositions comprising insulin and a GLP-1 peptide in one combined formulation. The present invention provides preparations with increased stability comprising a ligand for the His^{B10} anion sites as well as insulin and a GLP-1 peptide.

### BRIEF DESCRIPTION OF THE DRAWINGS

FIG. 13 shows a 600 MHz proton NMR spectrum of mixtures of aspart and liraglutide with constant aspart concentration 0.6 mM and varying concentrations of liraglutide.

FIG. 14 shows a 600 MHz proton NMR spectrum of mixtures of aspart and liraglutide in the presence of 3 mM 5-Benzyl-2H-tetrazole.

FIG. 15 shows a 600 MHz proton NMR spectrum of mixtures of aspart and liraglutide in the presence of 3 mM 5-Naphthalen-1-ylmethylenethiazolidine-2,4-dione.

FIGS. 1-3 show the stability (i.e. the tendency to fibrillate) for a formulation A consisting of: 1.2 mM liraglutide, 0.6 mM insulin aspart, 0.2 mM Zn²⁺ (corresponding to 2 Zn²⁺ ions per insulin hexamer),14 mg/ml propylene glycol, 60 mM phenol, 5 mM phosphate, pH 7.7.

FIGS. 4-7 show the stability for a formulation B consisting of: 2.4 mM insulin detemir, 1.6 mM Zn²⁺ (corresponding to 4 Zn²⁺ ions/insulin hexamer), 1.2 mM liraglutide, 14 mg/ml propylene glycol, 60 mM phenol, 5 mM phosphate, pH 7.7.

FIGS. 8-12 show the stability for a formulation C consisting of: 2.4 mM insulin detemir, 2.0 mM Zn²⁺ (corresponding to 5 Zn²⁺ ions/insulin hexamer), 1.2 mM liraglutide, 14 mg/ml propylene glycol, 60 mM phenol, 5 mM phosphate, pH 7.7.

### SUMMARY OF THE INVENTION

The present invention relates to pharmaceutical compositions comprising an insulinotropic peptide, an insulin peptide and a ligand for the His^{B10} anion site. These ligands may be selected from the group consisting of carboxylates, dithiocarboxylates, phenolates, thiophenolates, alkylthiolates, sulfonamides, imidazoles, triazoles, 4-cyano-1,2,3-triazoles, pyrimidine-2,4,6-triones, benzimidazoles, benzotriazoles, purines, thiazolidinediones, tetrazoles, 5-mercaptotetrazoles, rhodanines, N-hydroxyazoles, hydantoines, thiohydantoines, barbiturates, naphthoic acids, salicylic acids, salts containing SCN⁻ anions and salts containing Cl⁻ anions.

### DEFINITIONS

The following is a detailed definition of the terms used in the specification.

The term “effective amount” as used herein means a dosage which is sufficient in order for the treatment of the patient to be effective compared with no treatment.

The term “medicament” as used herein means a pharmaceutical composition suitable for administration of the pharmaceutically active compounds to a patient.

The term “pharmaceutical composition” as used herein means a product comprising one or more active compounds or a salt thereof together with pharmaceutical excipients such as buffer, preservative and tonicity modifier, said pharmaceutical composition being useful for treating, preventing or reducing the severity of a disease or disorder by administration of said pharmaceutical composition to a person. Thus a pharmaceutical composition is also known in the art as a pharmaceutical formulation.

The term “soluble pharmaceutical composition” as used herein means an insulinotropic peptide which is substantially soluble, and an insulin peptide which is substantially soluble in the combined composition. Thus, a predissolved soluble pharmaceutical composition will be substantially soluble, and a soluble pharmaceutical composition which is to be reconstituted will be substantially soluble once it has been dissolved in the prescribed reconstitution liquid. It is to be understood that pH of a pharmaceutical composition which is to be reconstituted is the pH value which is measured on the reconstituted composition produced by reconstitution in the prescribed reconstitution liquid at room temperature.

The term “pharmaceutically acceptable” as used herein means suited for normal pharmaceutical applications, i.e. giving rise to no adverse events in patients etc.

The term “buffer” as used herein refers to a chemical compound in a pharmaceutical composition that reduces the tendency of pH of the composition to change over time as would otherwise occur due to chemical reactions. Buffers include chemicals such as sodium phosphate, TRIS, HEPES, glycine and sodium citrate, or a mixture thereof.

The term “preservative” as used herein refers to a chemical compound which is added to a pharmaceutical composition to prevent or delay microbial activity (growth and metabolism). Examples of pharmaceutically acceptable preservatives are phenol, m-cresol and a mixture of phenol and m-cresol.

The term “isotonicity agent” as used refers to a chemical compound in a pharmaceutical composition that serves to modify the osmotic pressure of the pharmaceutical composition so that the osmotic pressure becomes closer to that of human plasma. Isotonicity agents include NaCl, glycerol, mannitol, sorbitol, propylene glycol or a mixture thereof. etc.

The term “stabilizer” as used herein refers to chemicals added to peptide containing pharmaceutical compositions in order to stabilize the peptide, i.e. to increase the shelf life and/or in-ude time of such compositions. Examples of stabilizers used in pharmaceutical formulations are L-glycine, L-histidine, arginine, polyethylene glycol, and carboxymethylcellulose.

The term “surfactant” as used herein refers to any substance, in particular a detergent, that can adsorb at surfaces and interfaces, like liquid to air, liquid to liquid, liquid to container or liquid to any solid. The surfactant may be selected from a detergent, ethoxylated castor oil, polyglycolyzed glycerides, acetylated monoglycerides, sorbitan fatty acid esters, polysorbate, such as polysorbate-20, poloxamers, such as poloxamer 188 and poloxamer 407, polyoxyethylene sorbitan fatty acid esters, polyoxyethylene derivatives such as alkylated and alkoxylated derivatives (tweens, e.g. Tween-20, or Tween-80), monoglycerides or ethoxylated derivatives thereof, diglycerides or polyoxyethylene derivatives thereof, glycerol, cholic acid or derivatives thereof, lecithins, alcohols and phospholipids, glycerophospholipids (lecithins, kephalins, phosphatidyl serine), glyceroglycolipids (galactopyransoide), sphingophospholipids (sphingomyelin), and sphingoglycolipids (ceramides, gangliosides), DSS (docusate sodium, CAS registry no [577-11-7]), docusate calcium, CAS registry no [128-49-4]), docusate potassium, CAS registry no [7491-09-0]), SDS (sodium dodecyl sulfate or sodium lauryl sulfate), dipalmitoyl phosphatidic acid, sodium caprylate, bile acids and salts thereof and glycine or taurine conjugates, ursodeoxycholic acid, sodium cholate, sodium deoxycholate, sodium taurocholate, sodium glycocholate, N-Hexadecyl-N,N-dimethyl-3-ammonio-1-propanesulfonate, anionic (alkyl-aryl-sulphonates) monovalent surfactants, palmitoyl lysophosphatidyl-L-serine, lysophospholipids (e.g. 1-acyl-sn-glycero-3-phosphate esters of ethanolamine, choline, serine or threonine), alkyl, alkoxyl (alkyl ester), alkoxy (alkyl ether)-derivatives of lysophosphatidyl and phosphatidylcholines, e.g. lauroyl and myristoyl derivatives of lysophosphatidylcholine, dipalmitoylphosphatidylcholine, and modifications of the polar head group, that is cholines, ethanolamines, phosphatidic acid, serines, threonines, glycerol, inositol, and the positively charged DODAC, DOTMA, DCP, BISHOP, lysophosphatidylserine and lysophosphatidylthreonine, zwitterionic surfactants (e.g. N-alkyl-N,N-dimethylammonio-1-propanesulfonates, 3-cholamido-1-propyldimethylammonio-1-propanesulfonate, dodecylphosphocholine, myristoyl lysophosphatidylcholine, hen egg lysolecithin), cationic surfactants (quarternary ammonium bases) (e.g. cetyl-trimethylammonium bromide, cetylpyridinium chloride), non-ionic surfactants, polyethyleneoxide/polypropyleneoxide block copolymers (Pluronics/Tetronics, Triton X-100, Dodecyl β-D-glucopyranoside) or polymeric surfactants (Tween-40, Tween-80, Brij-35), fusidic acid derivatives—(e.g. sodium tauro-dihydrofusidate etc.), long-chain fatty acids and salts thereof C6-C12 (eg. oleic acid and caprylic acid), acylcarnitines and derivatives, N^{α}-acylated derivatives of lysine, arginine or histidine, or side-chain acylated derivatives of lysine or arginine, N^{α}-acylated derivatives of dipeptides comprising any combination of lysine, arginine or histidine and a neutral or acidic amino acid, N^{α}-acylated derivative of a tripeptide comprising any combination of a neutral amino acid and two charged amino acids, or the surfactant may be selected from the group of imidazoline derivatives, or mixtures thereof.

The term “insulin peptide” as used herein means a peptide which is either human insulin or a chemically modified human insulin, such as an analogue or a derivative of human insulin.

The term “human insulin” as used herein means the human hormone whose structure and properties are well known, see e.g. DSHW Nicol and L F Smith: Nature, (1960) 4736:483-485, which is hereby incorporated by reference. Human insulin has two polypeptide chains that are connected by disulphide bridges between cysteine residues, namely the A-chain and the B-chain. The A-chain is a 21 amino acid peptide and the B-chain is a 30 amino acid peptide, the two chains being connected by three disulphide bridges: one between the cysteines in position 6 and 11 of the A-chain, the second between the cysteine in position 7 of the A-chain and the cysteine in position 7 of the B-chain, and the third between the cysteine in position 20 of the A-chain and the cysteine in position 19 of the B-chain.

The term “analogue” as used herein referring to a peptide means a modified peptide wherein one or more amino acid residues of the peptide have been substituted by other amino acid residues and/or wherein one or more amino acid residues have been deleted from the peptide and/or wherein one or more amino acid residues have been added to the peptide. Such addition or deletion of amino acid residues can take place at the N-terminal of the peptide and/or at the C-terminal of the peptide. By “one or more” is meant for example one, two, three, four, five, or up to ten.

The term “derivative” as used herein in relation to a parent peptide means a chemically modified parent protein or an analogue thereof, wherein at least one substituent is not present in the parent protein or an analogue thereof, i.e. a parent protein which has been covalently modified. Typical modifications are amides, carbohydrates, alkyl groups, acyl groups, esters, PEGylations and the like. Examples of derivatives of human insulin are threonine methyl ester^{B30} human insulin and N^{εB29}-tetradecanoyl des(B30) human insulin.

The term “GLP-1 compound” as used herein means GLP-1(7-37), which is well known in the art, as well as an insulinotropic analogue thereof and insulinotropic derivatives thereof. Non-limiting examples of GLP-1 analogues are GLP-1(7-36) amide, Arg³⁴-GLP-1(7-37), Gly⁸-GLP-1(7-37), Val⁸-GLP-1(7-36)-amide and Val⁸Asp²²-GLP-1(7-37). Non-limiting examples of GLP-1 derivatives are desamino-His⁷, Arg²⁶, Lys³⁴(N^{ε}-(γ-Glu(N^{α}-hexadecanoyl)))GLP-1(7-37), desamino-His⁷, Arg²⁶, Lys³⁴(N^{ε}-octanoyl)-GLP-1(7-37), Arg^{26,34}, Lys³⁸(N^{ε}-(ω-carboxypentadecanoyl))-GLP-1(7-38), Arg^{26,34}, Lys³⁶ (N^{ε}-(γ-Glu(N^{α}-hexadecanoyl)))-GLP-1(7-36) and Arg³⁴, Lys²⁶(N^{ε}-(γ-Glu(N^{α}-hexadecanoyl)))-GLP-1(7-37).

The term “stable GLP-1 compound” as used herein means a chemically modified GLP-1(7-37), i.e. an analogue or a derivative which exhibits an in vivo plasma elimination half-life of at least 10 hours in man, as determined by the following method. The method for determination of plasma elimination half-life of a peptide in man is: The compound is dissolved in an isotonic buffer, pH 7.4, PBS or any other suitable buffer. The dose is injected peripherally, preferably in the abdominal or upper thigh. Blood samples for determination of active compound are taken at frequent intervals, and for a sufficient duration to cover the terminal elimination part (e.g. Pre-dose, 1, 2, 3, 4, 5, 6, 7, 8, 10, 12, 24 (day 2), 36 (day 2), 48 (day 3), 60 (day 3), 72 (day 4) and 84 (day 4) hours post dose). Determination of the concentration of active compound is performed as described in Wilken et al., Diabetologia 43(51):A143, 2000. Derived pharmacokinetic parameters are calculated from the concentration-time data for each individual subject by use of non-compartmental methods, using the commercially available software WinNonlin Version 2.1 (Pharsight, Cary, N.C., USA). The terminal elimination rate constant is estimated by log-linear regression on the terminal log-linear part of the concentration-time curve, and used for calculating the elimination half-life.

The term “dipeptidyl aminopeptidase IV protected GLP-1 compound” as used herein means a GLP-1 compound which is more resistant to the plasma peptidase dipeptidyl aminopeptidase IV (DPP-IV) than the native GLP-1 agonist, GLP-1(7-37). Resistance of a GLP-1 compound towards degradation by dipeptidyl aminopeptidase IV is determined by the following degradation assay:

Aliquots of the GLP-1 compound (5 nmol) are incubated at 37° C. with 1 μL of purified dipeptidyl aminopeptidase IV corresponding to an enzymatic activity of 5 mU for 10-180 minutes in 100 μL of 0.1 M triethylamine-HCl buffer, pH 7.4. Enzymatic reactions are terminated by the addition of 5 μL of 10% trifluoroacetic acid, and the peptide degradation products are separated and quantified using HPLC analysis. One method for performing this analysis is: The mixtures are applied onto a Vydac C18 widepore (30 nm pores, 5 μm particles) 250×4.6 mm column and eluted at a flow rate of 1 ml/min with linear stepwise gradients of acetonitrile in 0.1% trifluoroacetic acid (0% acetonitrile for 3 min, 0-24% acetonitrile for 17 min, 24-48% acetonitrile for 1 min) according to Siegel et al., Regul. Pept. 1999; 79:93-102 and Mentlein et al. Eur. J. Biochem. 1993; 214:829-35. Peptides and their degradation products may be monitored by their absorbance at 220 nm (peptide bonds) or 280 nm (aromatic amino acids), and are quantified by integration of their peak areas related to those of standards. The rate of hydrolysis of a GLP-1 compound by dipeptidyl aminopeptidase IV is estimated at incubation times which result in less than 10% of the GLP-1 compound being hydrolysed.

The term “insulinotropic” as used herein referring to a peptide or a compound means the ability to stimulate secretion of insulin in response to an increased plasma glucose level. Insulinotropic peptides and compounds are agonists of the GLP-1 receptor. The insulinotropic property of a compound may be determined by in vitro or in vivo assays known in the art. The following in vitro assay may be used to determine the insulinotropic nature of a compound such as a peptide. Preferably insulinotropic compounds exhibit an EC₅₀ value in below assay of less than 5 nM, even more preferably EC50 values less than 500 pM.

Baby hamster kidney (BHK) cells expressing the cloned human GLP-1 receptor (BHK 467-12A) are grown in DMEM media with the addition of 100 IU/mL penicillin, 100 μL/mL streptomycin, 10% foetal calf serum and 1 mg/mL Geneticin G-418 (Life Technologies). Plasma membranes are prepared by homogenization in buffer (10 mM Tris-HCl, 30 mM NaCl and 1 mM dithiothreitol, pH 7.4, containing, in addition, 5 mg/mL leupeptin (Sigma), 5 mg/L pepstatin (Sigma), 100 mg/L bacitracin (Sigma), and 16 mg/L aprotinin (Calbiochem-Novabiochem, La Jolla, Calif.)). The homogenate was centrifuged on top of a layer of 41% W7v sucrose. The white band between the two layers was diluted in buffer and centrifuged. Plasma membranes were stored at −80° C. until used.

The functional receptor assay is carried out by measuring cAMP as a response to stimulation by the insulinotropic peptide or insulinotropic compound. Incubations are carried out in 96-well microtiter plates in a total volume of 140 mL and with the following final concentrations: 50 mM Tris-HCl, 1 mM EGTA, 1.5 mM MgSO₄, 1.7 mM ATP, 20 mM GTP, 2 mM 3-isobutyl-1-methylxanthine (IBMX), 0.01% w/v Tween-20, pH 7.4. Compounds are dissolved and diluted in buffer. GTP is freshly prepared for each experiment: 2.5 μg of membrane is added to each well and the mixture is incubated for 90 min at room temperature in the dark with shaking. The reaction is stopped by the addition of 25 mL 0.5 M HCl. Formed cAMP is measured by a scintillation proximity assay (RPA 542, Amersham, UK). A dose-response curves is plotted for the compound and the EC₅₀ value is calculated using GraphPad Prism software.

The term “prodrug of an insulinotropic compound” as used herein means a chemically modified compound which following administration to the patient is converted to an insulinotropic compound. Such prodrugs are typically amino acid extended versions or esters of an insulinotropic compound.

The term “exendin-4 compound” as used herein is defined as exendin-4(1-39), which is well known in the art, insulinotropic fragments thereof, insulinotropic analogs thereof and insulinotropic derivatives thereof. Insulinotropic fragments of exendin-4 are insulinotropic peptides for which the entire sequence can be found in the sequence of exendin-4 and where at least one terminal amino acid has been deleted. Examples of insulinotropic fragments of exendin-4(1-39) are exendin-4(1-38) and exendin-4(1-31). The insulinotropic property of a compound may be determined by in vivo or in vitro assays well known in the art. For instance, the compound may be administered to an animal and monitoring the insulin concentration over time. Insulinotropic analogs of exendin-4(1-39) refer to the respective molecules wherein one or more of the amino acids residues have been exchanged with other amino acid residues and/or from which one or more amino acid residues have been deleted and/or from which one or more amino acid residues have been added with the proviso that said analogue either is insulinotropic or is a prodrug of an insulinotropic compound. An example of an insulinotropic analog of exendin-4(1-39) is Ser²Asp³-exendin-4(1-39) wherein the amino acid residues in position 2 and 3 have been replaced with serine and aspartic acid, respectively (this particular analog also being known in the art as exendin-3). Insulinotropic derivatives of exendin-4(1-39) and analogs thereof are what the person skilled in the art considers to be derivatives of these peptides, i.e. having at least one substituent which is not present in the parent peptide molecule with the proviso that said derivative either is insulinotropic or is a prodrug of an insulinotropic compound. Examples of substituents are amides, carbohydrates, alkyl groups, esters and lipophilic substituents. An example of an insulinotropic derivatives of exendin-4(1-39) and analogs thereof is Tyr³¹-exendin-4(1-31)-amide.

The term “stable exendin-4 compound” as used herein means a chemically modified exendin-4(1-39), i.e. an analogue or a derivative which exhibits an in vivo plasma elimination half-life of at least 10 hours in man, as determined by the method described under the definition of “stable GLP-1 compound”.

The term “dipeptidyl aminopeptidase IV protected exendin-4 compound” as used herein means an exendin-4 compound which is more resistant towards the plasma peptidase dipeptidyl aminopeptidase IV (DPP-IV) than exendin-4, as determined by the assay described under the definition of dipeptidyl aminopeptidase IV protected GLP-1 compound.

The term “isoelectric point” as used herein means the pH value where the overall net charge of a macromolecule such as a peptide is zero. In peptides there may be several charged groups, and at the isoelectric point the sum of all these charges is zero. At a pH above the isoelectric point the overall net charge of the peptide will be negative, whereas at pH values below the isoelectric point the overall net charge of the peptide will be positive.

The term “reconstituted” as used herein referring to a pharmaceutical composition means an aqueous composition which has been formed by the addition of water to a solid material comprising the active pharmaceutical ingredient. Pharmaceutical compositions for reconstitution are applied where a liquid composition with acceptable shelf-life cannot be produced. An example of a reconstituted pharmaceutical composition is the solution which results when adding water to a freeze dried composition. The solution is often for parenteral administration and thus water for injection is typically used for reconstituting the solid material.

The term “about” as used herein in relation to the concentration of a peptide in a pharmaceutical composition means plus or minus 10%. Hence, the concentration “about 5 mg/mL insulin” means a concentration of 4.5 mg/mL insulin to 5.5 mg/mL insulin.

“Halogen” designates an atom selected from the group consisting of F, Cl, Br and I.

The term “C₁-C₆-alkyl” as used herein represents a saturated, branched or straight hydrocarbon group having from 1 to 6 carbon atoms. Representative examples include, but are not limited to, methyl, ethyl, n-propyl, isopropyl, butyl, isobutyl, sec-butyl, tert-butyl, n-pentyl, isopentyl, neopentyl, tert-pentyl, n-hexyl, isohexyl and the like.

The term “C₁-C₆-alkylene” as used herein represents a saturated, branched or straight bivalent hydrocarbon group having from 1 to 6 carbon atoms. Representative examples include, but are not limited to, methylene, 1,2-ethylene, 1,3-propylene, 1,2-propylene, 1,4-butylene, 1,5-pentylene, 1,6-hexylene, and the like.

The term “C₂-C₆-alkenyl” as used herein represents a branched or straight hydrocarbon group having from 2 to 6 carbon atoms and at least one double bond. Examples of such groups include, but are not limited to, vinyl, 1-propenyl, 2-propenyl, iso-propenyl, 1,3-butadienyl, 1-butenyl, 2-butenyl, 3-butenyl, 2-methyl-1-propenyl, 1-pentenyl, 2-pentenyl, 3-pentenyl, 4-pentenyl, 3-methyl-2-butenyl, 1-hexenyl, 2-hexenyl, 3-hexenyl, 2,4-hexadienyl, 5-hexenyl and the like.

The term “C₂-C₆-alkynyl” as used herein represents a branched or straight hydrocarbon group having from 2 to 6 carbon atoms and at least one triple bond. Examples of such groups include, but are not limited to, ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, 5-hexynyl, 2,4-hexadiynyl and the like.

The term “C₁-C₆-alkoxy” as used herein refers to the radical —O—C₁-C₆-alkyl, wherein C₁-C₆-alkyl is as defined above. Representative examples are methoxy, ethoxy, n-propoxy, isopropoxy, butoxy, sec-butoxy, tert-butoxy, pentoxy, isopentoxy, hexoxy, isohexoxy and the like.

The term “C₃-C₈-cycloalkyl” as used herein represents a saturated, carbocyclic group having from 3 to 8 carbon atoms. Representative examples are cyclopropyl, cyclobutyl, cyclopentyl, cyclohexyl, cycloheptyl, cyclooctyl and the like.

The term “C₄₋₈-cycloalkenyl” as used herein represents a non-aromatic, carbocyclic group having from 4 to 8 carbon atoms containing one or two double bonds. Representative examples are 1-cyclopentenyl, 2-cyclopentenyl, 3-cyclopentenyl, 1-cyclohexenyl, 2-cyclohexenyl, 3-cyclohexenyl, 2-cycloheptenyl, 3-cycloheptenyl, 2-cyclooctenyl, 1,4-cyclooctadienyl and the like.

The term “heterocyclyl” as used herein represents a non-aromatic 3 to 10 membered ring containing one or more heteroatoms selected from nitrogen, oxygen and sulphur and optionally containing one or two double bonds. Representative examples are pyrrolidinyl, piperidyl, piperazinyl, morpholinyl, thiomorpholinyl, aziridinyl, tetrahydrofuranyl and the like.

The term “aryl” as used herein is intended to include carbocyclic, aromatic ring systems such as 6 membered monocyclic and 9 to 14 membered bi- and tricyclic, carbocyclic, aromatic ring systems. Representative examples are phenyl, biphenylyl, naphthyl, anthracenyl, phenanthrenyl, fluorenyl, indenyl, azulenyl and the like. Aryl is also intended to include the partially hydrogenated derivatives of the ring systems enumerated above. Non-limiting examples of such partially hydrogenated derivatives are 1,2,3,4-tetrahydronaphthyl, 1,4-dihydronaphthyl and the like.

The term “arylene” as used herein is intended to include divalent, carbocyclic, aromatic ring systems such as 6 membered monocyclic and 9 to 14 membered bi- and tricyclic, divalent, carbocyclic, aromatic ring systems. Representative examples are phenylene, biphenylylene, naphthylene, anthracenylene, phenanthrenylene, fluorenylene, indenylene, azulenylene and the like. Arylene is also intended to include the partially hydrogenated derivatives of the ring systems enumerated above. Non-limiting examples of such partially hydrogenated derivatives are 1,2,3,4-tetrahydronaphthylene, 1,4-dihydronaphthylene and the like.

The term “aryloxy” as used herein denotes a group —O-aryl, wherein aryl is as defined above.

The term “aroyl” as used herein denotes a group —C(O)-aryl, wherein aryl is as defined above.

The term “heteroaryl” as used herein is intended to include aromatic, heterocyclic ring systems containing one or more heteroatoms selected from nitrogen, oxygen and sulphur such as 5 to 7 membered monocyclic and 8 to 14 membered bi- and tricyclic aromatic, heterocyclic ring systems containing one or more heteroatoms selected from nitrogen, oxygen and sulphur. Representative examples are furyl, thienyl, pyrrolyl, pyrazolyl, 3-oxopyrazolyl, oxazolyl, thiazolyl, imidazolyl, isoxazolyl, isothiazolyl, 1,2,3-triazolyl, 1,2,4-triazolyl, pyranyl, pyridyl, pyridazinyl, pyrimidinyl, pyrazinyl, 1,2,3-triazinyl, 1,2,4-triazinyl, 1,3,5-triazinyl, 1,2,3-oxadiazolyl, 1,2,4-oxadiazolyl, 1,2,5-oxadiazolyl, 1,3,4-oxadiazolyl, 1,2,3-thiadiazolyl, 1,2,4-thiadiazolyl, 1,2,5-thiadiazolyl, 1,3,4-thiadiazolyl, tetrazolyl, thiadiazinyl, indolyl, isoindolyl, benzofuryl, benzothienyl, indazolyl, benzimidazolyl, benzthiazolyl, benzisothiazolyl, benzoxazolyl, benzisoxazolyl, purinyl, quinazolinyl, quinolizinyl, quinolinyl, isoquinolinyl, quinoxalinyl, naphthyridinyl, pteridinyl, carbazolyl, azepinyl, diazepinyl, acridinyl, thiazolidinyl, 2-thiooxothiazolidinyl and the like. Heteroaryl is also intended to include the partially hydrogenated derivatives of the ring systems enumerated above. Non-limiting examples of such partially hydrogenated derivatives are 2,3-dihydrobenzofuranyl, pyrrolinyl, pyrazolinyl, indolinyl, oxazolidinyl, oxazolinyl, oxazepinyl and the like.

The term “heteroarylene” as used herein is intended to include divalent, aromatic, heterocyclic ring systems containing one or more heteroatoms selected from nitrogen, oxygen and sulphur such as 5 to 7 membered monocyclic and 8 to 14 membered bi- and tricyclic aromatic, heterocyclic ring systems containing one or more heteroatoms selected from nitrogen, oxygen and sulphur. Representative examples are furylene, thienylene, pyrrolylene, oxazolylene, thiazolylene, imidazolylene, isoxazolylene, isothiazolylene, 1,2,3-triazolylene, 1,2,4-triazolylene, pyranylene, pyridylene, pyridazinylene, pyrimidinylene, pyrazinylene, 1,2,3-triazinylene, 1,2,4-triazinylene, 1,3,5-triazinylene, 1,2,3-oxadiazolylene, 1,2,4-oxadiazolylene, 1,2,5-oxadiazolylene, 1,3,4-oxadiazolylene, 1,2,3-thiadiazolylene, 1,2,4-thiadiazolylene, 1,2,5-thiadiazolylene, 1,3,4-thiadiazolylene, tetrazolylene, thiadiazinylene, indolylene, isoindolylene, benzofurylene, benzothienylene, indazolylene, benzimidazolylene, benzthiazolylene, benzisothiazolylene, benzoxazolylene, benzisoxazolylene, purinylene, quinazolinylene, quinolizinylene, quinolinylene, isoquinolinylene, quinoxalinylene, naphthyridinylene, pteridinylene, carbazolylene, azepinylene, diazepinylene, acridinylene and the like. Heteroaryl is also intended to include the partially hydrogenated derivatives of the ring systems enumerated above. Non-limiting examples of such partially hydrogenated derivatives are 2,3-dihydrobenzofuranylene, pyrrolinylene, pyrazolinylene, indolinylene, oxazolidinylene, oxazolinylene, oxazepinylene and the like.

The term “ArG1” as used herein is intended to include an aryl or arylene radical as applicable, where aryl or arylene are as defined above but limited to phenyl, biphenylyl, naphthyl, anthracenyl, phenanthrenyl, fluorenyl, indenyl, and azulenyl as well as the corresponding divalent radicals.

The term “ArG2” as used herein is intended to include an aryl or arylene radical as applicable, where aryl or arylene are as defined above but limited to phenyl, biphenylyl, naphthyl, fluorenyl, and indenyl, as well as the corresponding divalent radicals.

The term “Het1” as used herein is intended to include a heteroaryl or heteroarylene radical as applicable, where heteroaryl or heteroarylene are as defined above but limited to furyl, thienyl, pyrrolyl, pyrazolyl, 3-oxopyrazolyl, oxazolyl, thiazolyl, imidazolyl, isoxazolyl, isothiazolyl, 1,2,3-triazolyl, 1,2,4-triazolyl, pyranyl, pyridyl, pyridazinyl, pyrimidinyl, pyrazinyl, 1,2,3-triazinyl, 1,2,4-triazinyl, 1,3,5-triazinyl, 1,2,3-oxadiazolyl, 1,2,4-oxadiazolyl, 1,2,5-oxadiazolyl, 1,3,4-oxadiazolyl, 1,2,3-thiadiazolyl, 1,2,4-thiadiazolyl, 1,2,5-thiadiazolyl, 1,3,4-thiadiazolyl, tetrazolyl, thiadiazinyl, indolyl, isoindolyl, benzofuryl, benzothienyl, indazolyl, benzimidazolyl, benzthiazolyl, benzisothiazolyl, benzoxazolyl, benzisoxazolyl, purinyl, quinazolinyl, quinolizinyl, quinolinyl, isoquinolinyl, quinoxalinyl, naphthyridinyl, pteridinyl, carbazolyl, azepinyl, diazepinyl, acridinyl, thiazolidinyl, 2-thiooxothiazolidinyl, as well as the corresponding divalent radicals.

The term “Het2” as used herein is intended to include a heteroaryl or heteroarylene radical as applicable, where heteroaryl or heteroarylene are as defined above but limited to furyl, thienyl, pyrrolyl, pyrazolyl, 3-oxopyrazolyl, oxazolyl, thiazolyl, imidazolyl, isoxazolyl, isothiazolyl, 1,2,3-triazolyl, 1,2,4-triazolyl, pyranyl, pyridyl, pyridazinyl, pyrimidinyl, pyrazinyl, 1,2,3-triazinyl, 1,2,4-triazinyl, 1,3,5-triazinyl, 1,2,3-oxadiazolyl, 1,2,4-oxadiazolyl, 1,2,5-oxadiazolyl, 1,3,4-oxadiazolyl, 1,2,3-thiadiazolyl, 1,2,4-thiadiazolyl, 1,2,5-thiadiazolyl, 1,3,4-thiadiazolyl, tetrazolyl, thiadiazinyl, indolyl, isoindolyl, benzofuryl, benzothienyl, benzimidazolyl, benzthiazolyl, benzisothiazolyl, benzoxazolyl, benzisoxazolyl, quinolinyl, isoquinolinyl, quinoxalinyl, carbazolyl, thiazolidinyl, 2-thiooxothiazolidinyl, as well as the corresponding divalent radicals.

The term “Het3” as used herein is intended to include a heteroaryl or heteroarylene radical as applicable, where heteroaryl or heteroarylene are as defined above but limited to furyl, thienyl, pyrrolyl, pyrazolyl, 3-oxopyrazolyl, oxazolyl, thiazolyl, imidazolyl, isoxazolyl, isothiazolyl, 1,2,3-triazolyl, 1,2,4-triazolyl, pyridyl, tetrazolyl, indolyl, isoindolyl, benzofuryl, benzothienyl, benzimidazolyl, benzthiazolyl, benzisothiazolyl, benzoxazolyl, benzisoxazolyl, quinolyl, isoquinolyl, quinoxalinyl, carbazolyl, thiazolidinyl, 2-thiooxothiazolidinyl, as well as the corresponding divalent radicals.

“Aryl-C₁-C₆-alkyl”, “heteroaryl-C₁-C₆-alkyl”, “aryl-C₂-C₆-alkenyl” etc. is intended to mean C₁-C₆-alkyl or C₂-C₆-alkenyl as defined above, substituted by an aryl or heteroaryl as defined above, for example:

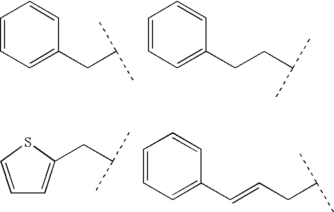

The term “optionally substituted” as used herein means that the groups in question are either unsubstituted or substituted with one or more of the substituents specified. When the groups in question are substituted with more than one substituent the substituents may be the same or different.

Certain of the above defined terms may occur more than once in the structural formulae, and upon such occurrence each term shall be defined independently of the other.

Furthermore, when using the terms “independently are” and “independently selected from” it should be understood that the groups in question may be the same or different.

The terms “treatment” and “treating” as used herein means the management and care of a patient for the purpose of combating a disease, disorder or condition. The term is intended to include the delaying of the progression of the disease, disorder or condition, the alleviation or relief of symptoms and complications, and/or the cure or elimination of the disease, disorder or condition. The patient to be treated is preferably a mammal, in particular a human being.

When in the specification or claims mention is made of groups of compounds such as carboxylates, dithiocarboxylates, phenolates, thiophenolates, alkylthiolates, sulfonamides, imidazoles, triazoles, 4-cyano-1,2,3-triazoles, benzimidazoles, benzotriazoles, purines, thiazolidinediones, tetrazoles, 5-mercaptotetrazoles, rhodanines, N-hydroxyazoles, hydantoines, thiohydantoines, naphthoic acids and salicylic acids, these groups of compounds are intended to include also derivatives of the compounds from which the groups take their name.

### DESCRIPTION OF THE INVENTION

The present invention is concerned with pharmaceutical compositions comprising an insulinotropic peptide, an insulin peptide and a ligand for the His^{B10} anion site. A combination formulation comprising these elements with a fixed ratio of the two peptides be a very efficacious treatment as well as one requiring less injections when administered to the same patient.

Thus it is one object of this invention to provide stable compositions comprising the two peptides but such mixtures may present problems in terms of insufficient stability of the combined preparation. Thus, there is a big need for stable pharmaceutical compositions comprising an insulin peptide and an insulinotropic peptide in one combined formulation. The demonstrated binding of several ligands to the Zn-binding pocket of the insulin peptide helps to stabilize the R₆ conformation.

It has surprisingly been found that the interaction between the insulin peptide and an insulinotropic peptide that takes place in the solution phase is avoided by the presence in the composition of a ligand for the His^{B10} anion site as defined herein. Examples of ligands for the His^{B10} anion site can be seen in WO 2004/056347 (Novo Nordisk), page 86-370, examples 1-1010, which are hereby incorporated by reference.

Further, it has been found that a surprising increased stability occurs when adding both a surfactant and a ligand for the His^{B10} anion sites to the pharmaceutical composition of the invention.

The present invention is further described by the following non-limiting embodiments:

Embodiment 0. A pharmaceutical composition comprising an insulinotropic peptide, an insulin peptide and a ligand for the His^{B10} anion site selected from the group consisting of carboxylates, dithiocarboxylates, phenolates, thiophenolates, alkylthiolates, sulfonamides, imidazoles, triazoles, 4-cyano-1,2,3-triazoles, pyrimidine-2,4,6-triones, benzimidazoles, benzotriazoles, purines, thiazolidinediones, tetrazoles, 5-mercaptotetrazoles, rhodanines, N-hydroxyazoles, hydantoines, thiohydantoines, barbiturates, naphthoic acids, salicylic acids, salts containing SCN⁻ anions and salts containing Cl⁻ anions.
Embodiment 1. A pharmaceutical composition comprising an insulinotropic peptide, an insulin peptide and a ligand for the His^{B10} anion site selected from the group consisting of carboxylates, dithiocarboxylates, phenolates, thiophenolates, alkylthiolates, sulfonamides, imidazoles, triazoles, 4-cyano-1,2,3-triazoles, pyrimidine-2,4,6-triones, benzimidazoles, benzotriazoles, purines, thiazolidinediones, tetrazoles, 5-mercaptotetrazoles, rhodanines, N-hydroxyazoles, hydantoines, thiohydantoines, barbiturates, naphthoic acids and salicylic acids.
Embodiment 2. A pharmaceutical composition according to embodiment 1 wherein the ligand for the His^{B10} anion site is

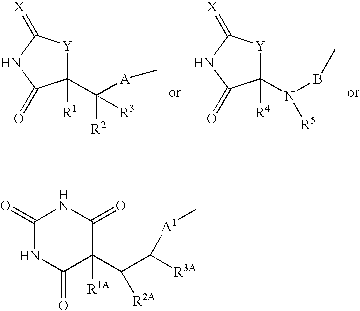

wherein

### X is ═O, ═S or ═NH

### Y is —S—, —O— or —NH—

R¹, R_{1A} and R⁴ are independently selected from hydrogen or C₁-C₆-alkyl,
R² and R^{2A} are hydrogen or C₁-C₆-alkyl or aryl, R¹ and R² may optionally be combined to form a double bond, R^{1A} and R^{2A} may optionally be combined to form a double bond,
R³, R^{3A} and R⁵ are independently selected from hydrogen, halogen, aryl optionally substituted with one or more substituents independently selected from R¹⁶, C₁-C₆-alkyl, or —C(O)NR¹¹R¹²,
A, A¹ and B are independently selected from C₁-C₆-alkyl, aryl, aryl-C₁-C₆-alkyl, —NR¹¹-aryl, aryl-C₂-C₆-alkenyl or heteroaryl, wherein the alkyl or alkenyl is optionally substituted with one or more substituents independently selected from R⁶ and the aryl or heteroaryl is optionally substituted with up to four substituents R⁷, R⁸, R⁹, and R¹⁰,
A and R³ may be connected through one or two valence bonds, B and R⁵ may be connected through one or two valence bonds,
R⁶ is independently selected from halogen, —CN, —CF₃, —OCF₃, aryl, —COOH and —NH₂,
R⁷, R⁸, R⁹ and R¹⁰ are independently selected from
hydrogen, halogen, —CN, —CH₂CN, —CHF₂, —CF₃, —OCF₃, —OCHF₂, —OCH₂CF₃, —OCF₂CHF₂, —S(O)₂CF₃, —OS(O)₂CF₃, —SCF₃, —NO₂, —OR¹¹, —NR¹¹R¹², —SR¹¹, —NR¹¹S(O)₂R¹², —S(O)₂NR¹¹R¹², —S(O)NR¹¹R¹², —S(O)R¹¹, —S(O)₂R¹¹, —OS(O)₂R¹¹, —C(O)NR¹¹R¹², —OC(O)NR¹¹R¹², —NR¹¹C(O)R¹², —CH₂C(O)NR¹¹R¹², —OC₁-C₆-alkyl-C(O)NR¹¹R¹², —CH₂OR¹¹, —CH₂OC(O)R¹¹—CH₂NR¹¹R¹², —OC(O)R¹¹, —OC₁-C₁₅-alkyl-C(O)OR¹¹, —OC₁-C₆-alkyl-OR¹¹, —SC₁-C₆-alkyl-C(O)OR¹¹, —C₂-C₆-alkenyl-C(═O)OR¹¹, —NR¹¹—C(═O)—C₁-C₆-alkyl-C(═O)OR¹¹, —NR¹¹—C(═O)—C₁-C₆-alkenyl-C(═O)OR¹¹, —C(O)OR¹¹, C(O)R¹¹, or —C₂-C₆-alkenyl-C(═O)R¹¹, ═O, or —C₂-C₆-alkenyl-C(═O)—NR¹¹R¹²,
C₁-C₆-alkyl, C₂-C₆-alkenyl or C₂-C₆-alkynyl, each of which may optionally be substituted with one or more substituents independently selected from R¹³,
aryl, aryloxy, aryloxycarbonyl, aroyl, arylsulfanyl, aryl-C₁-C₆-alkoxy, aryl-C₁-C₆-alkyl, aryl-C₂-C₆-alkenyl, aroyl-C₂-C₆-alkenyl, aryl-C₂-C₆-alkynyl, heteroaryl, heteroaryl-C₁-C₆-alkyl, heteroaryl-C₂-C₆-alkenyl, heteroaryl-C₂-C₆-alkynyl, or C₃-C₆ cycloalkyl,
of which each cyclic moiety may optionally be substituted with one or more substituents independently selected from R¹⁴,
   R¹¹ and R¹² are independently selected from hydrogen, OH, C₁-C₂₀-alkyl, aryl-C₁-C₆-alkyl or aryl, wherein the alkyl groups may optionally be substituted with one or more substituents independently selected from R¹⁵, and the aryl groups may optionally be substituted one or more substituents independently selected from R¹⁶; R¹¹ and R¹² when attached to the same nitrogen atom may form a 3 to 8 membered heterocyclic ring with the said nitrogen atom, the heterocyclic ring optionally containing one or two further heteroatoms selected from nitrogen, oxygen and sulphur, and optionally containing one or two double bonds,
   R¹³ is independently selected from halogen, —CN, —CF₃, —OCF₃, —OR¹¹, —C(O)OR¹¹, —NR¹¹R¹², and —C(O)NR¹¹R¹²,
   R¹⁴ is independently selected from halogen, —C(O)OR¹¹, —CH₂C(O)OR¹¹, —CH₂OR¹¹, —CN, —CF₃, —OCF₃, —NO₂, —OR¹¹, —NR¹¹R¹², —NR¹¹C(O)R¹¹, —S(O)₂R¹¹, aryl and C₁-C₆-alkyl,
   R¹⁵ is independently selected from halogen, —CN, —CF₃, ═O, —OCF₃, —OC₁-C₆-alkyl, —C(O)OC₁-C₆-alkyl, —COOH and —NH₂,
   R¹⁶ is independently selected from halogen, —C(O)OC₁-C₆-alkyl, —COOH, —CN, —CF₃, —OCF₃, —NO₂, —OH, —OC₁-C₆-alkyl, —NH₂, C(═O) or C₁-C₆-alkyl, or any enantiomer, diastereomer, including a racemic mixture, tautomer as well as a salt thereof with a pharmaceutically acceptable acid or base.
   Embodiment 3. A pharmaceutical composition according to embodiment 2 wherein X is ═O or ═S.
   Embodiment 4. A pharmaceutical composition according to embodiment 3 wherein X is ═O.
   Embodiment 5. A pharmaceutical composition according to embodiment 3 wherein X is ═S.
   Embodiment 6. A pharmaceutical composition according to any one of the embodiments 2 to 5 wherein Y is —O— or —S—.
   Embodiment 7. A pharmaceutical composition according to embodiment 6 wherein Y is —O—.
   Embodiment 8. A pharmaceutical composition according to embodiment 6 wherein Y is —NH—.
   Embodiment 9. A pharmaceutical composition according to embodiment 6 wherein Y is —S—.
   Embodiment 10. A pharmaceutical composition according to any one of the embodiments 2 to 9 wherein A is aryl optionally substituted with up to four substituents, R⁷, R⁸, R⁹, and R¹⁰ which may be the same or different.
   Embodiment 11. A pharmaceutical composition according to embodiment 10 wherein A is selected from ArG1 optionally substituted with up to four substituents, R⁷, R⁸, R⁹, and R¹⁰ which may be the same or different.
   Embodiment 12. A pharmaceutical composition according to embodiment 11 wherein A is phenyl or naphtyl optionally substituted with up to four substituents, R⁷, R⁸, R⁹, and R¹⁰ which may be the same or different.
   Embodiment 13. A pharmaceutical composition according to embodiment 12 wherein A is

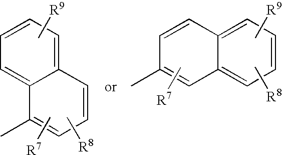

Embodiment 14. A pharmaceutical composition according to embodiment 12 wherein A is phenyl.
Embodiment 15. A pharmaceutical composition according to any one of the embodiments 2 to 9 wherein A is heteroaryl optionally substituted with up to four substituents, R⁷, R⁸, R⁹, and R¹⁰ which may be the same or different.
Embodiment 16. A pharmaceutical composition according to embodiment 15 wherein A is selected from Het1 optionally substituted with up to four substituents, R⁷, R⁸, R⁹, and R¹⁰ which may be the same or different.
Embodiment 17. A pharmaceutical composition according to embodiment 16 wherein A is selected from Het2 optionally substituted with up to four substituents, R⁷, R⁸, R⁹, and R¹⁰ which may be the same or different.
Embodiment 18. A pharmaceutical composition according to embodiment 17 wherein A is selected from Het3 optionally substituted with up to four substituents, R⁷, R⁸, R⁹, and R¹⁰ which may be the same or different.
Embodiment 19. A pharmaceutical composition according to embodiment 18 wherein A is selected from the group consisting of indolyl, benzofuranyl, quinolyl, furyl, thienyl, or pyrrolyl, wherein each heteroaryl may optionally substituted with up to four substituents, R⁷, R⁸, R⁹, and R¹⁰ which may be the same or different.
Embodiment 20. A pharmaceutical composition according to embodiment 18 wherein A is benzofuranyl optionally substituted with up to four substituents R⁷, R⁸, R⁹, and R¹⁰ which may be the same or different.
Embodiment 21. A pharmaceutical composition according to embodiment 20 wherein A is

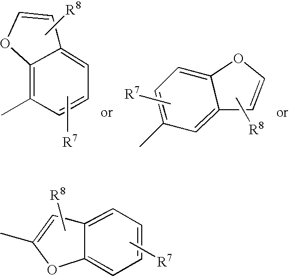

22. A pharmaceutical composition according to embodiment 18 wherein A is carbazolyl optionally substituted with up to four substituents R⁷, R⁸, R⁹, and R¹⁰ which may be the same or different.
Embodiment 23. A pharmaceutical composition according to embodiment 22 wherein A is

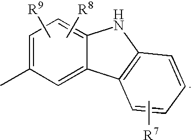

Embodiment 24. A pharmaceutical composition according to embodiment 18 wherein A is quinolyl optionally substituted with up to four substituents R⁷, R⁸, R⁹, and R¹⁰ which may be the same or different.
Embodiment 25. A pharmaceutical composition according to embodiment 24 wherein A is

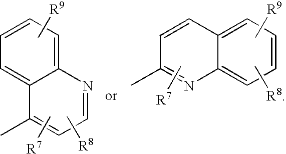

Embodiment 26. A pharmaceutical composition according to embodiment 18 wherein A is indolyl optionally substituted with up to four substituents R⁷, R⁸, R⁹, and R¹⁰ which may be the same or different.
Embodiment 27. A pharmaceutical composition according to embodiment 26 wherein A is

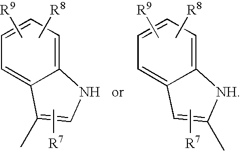

Embodiment 28. A pharmaceutical composition according to any one of the embodiments 2 to 27 wherein R¹ is hydrogen.
Embodiment 29. A pharmaceutical composition according to any one of the embodiments 2 to 28 wherein R² is hydrogen.
Embodiment 30. A pharmaceutical composition according to any one of the embodiments 2 to 27 wherein R¹ and R² are combined to form a double bond.
Embodiment 31. A pharmaceutical composition according to any one of the embodiments 2 to 30 wherein R³ is C₁-C₆-alkyl, halogen, or C(O)NR¹⁶R¹⁷.
Embodiment 32. A pharmaceutical composition according to embodiment 31 wherein R³ is C₁-C₆-alkyl or C(O)NR¹⁶R¹⁷.
Embodiment 33. A pharmaceutical composition according to embodiment 32 wherein R³ is methyl.
Embodiment 34. A pharmaceutical composition according to any one of the embodiments 2 to 9 wherein B is phenyl optionally substituted with up to four substituents, R⁷, R⁸, R⁹, and R¹⁰ which may be the same or different.
Embodiment 35. A pharmaceutical composition according to any one of the embodiments 2 to 9 or 34 wherein R⁴ is hydrogen.
Embodiment 36. A pharmaceutical composition according to any one of the embodiments 2 to 9 or 34 to 35 wherein R⁵ is hydrogen.
Embodiment 37. A pharmaceutical composition according to any one of the embodiments 2 to 36 wherein R⁶ is aryl.
Embodiment 38. A pharmaceutical composition according to embodiment 37 wherein R⁶ is phenyl.
Embodiment 39. A pharmaceutical composition according to any one of the embodiments 2 to 38 wherein R⁷, R⁸, R⁹ and R¹⁰ are independently selected from
hydrogen, halogen, —NO₂, —R¹¹—NR¹¹R¹², —SR¹¹, —NR¹¹S(O)₂R¹², —S(O)₂NR¹¹R¹², —S(O)NR¹¹R¹², —S(O)R¹¹—S(O)₂R¹¹—OS(O)₂R¹¹, —NR¹¹C(O)R¹²—CH₂OR¹¹, CH₂OC(O)R¹¹, —CH₂NR¹¹R¹², —OC(O)R¹¹, —OC₁-C₆-alkyl-C(O)OR¹¹, —OC₁-C₆-alkyl-C(O)NR¹¹R¹², —OC₁-C₆-alkyl-OR¹¹, —SC₁-C₆-alkyl-C(O)OR¹¹, —C₂-C₆-alkenyl-C(═O)OR¹¹, —C(O)OR¹¹, or —C₂-C₆-alkenyl-C(═O)R¹¹,
C₁-C₆-alkyl, C₂-C₆-alkenyl or C₂-C₆-alkynyl, which may each optionally be substituted with one or more substituents independently selected from R¹³
aryl, aryloxy, aroyl, arylsulfanyl, aryl-C₁-C₆-alkoxy, aryl-C₁-C₆-alkyl, aryl-C₂-C₆-alkenyl, aroyl-C₂-C₆-alkenyl, aryl-C₂-C₆-alkynyl, heteroaryl, heteroaryl-C₁-C₆-alkyl, wherein each of the cyclic moieties optionally may be substituted with one or more substituents independently selected from R¹⁴.
   Embodiment 40. A pharmaceutical composition according to embodiment 39 wherein R⁷, R⁸, R⁹ and R¹⁰ are independently selected from
hydrogen, halogen, —NO₂, —OR¹¹, —NR¹¹R¹², —SR¹¹, —S(O)₂R¹¹, —OS(O)₂R¹¹, CH₂OC(O)R¹¹, —OC(O)R¹¹, —OC₁-C₆-alkyl-C(O)OR¹¹, —OC₁-C₆-alkyl-OR¹¹, —SC₁-C₆-alkyl-C(O)OR¹¹, —C(O)OR¹¹, or —C₂-C₈-alkenyl-C(═O)R¹¹,
C₁-C₆-alkyl or C₁-C₆-alkenyl which may each optionally be substituted with one or more substituents independently selected from R¹³
aryl, aryloxy, aroyl, aryl-C₁-C₆-alkoxy, aryl-C₁-C₆-alkyl, heteroaryl,
of which each of the cyclic moieties optionally may be substituted with one or more substituents independently selected from R¹⁴.
   Embodiment 41. A pharmaceutical composition according to embodiment 40 wherein R⁷, R⁸, R⁹ and R¹⁰ are independently selected from
hydrogen, halogen, —NO₂, —OR¹¹, —NR¹¹R¹², —SR¹¹, —S(O)₂R¹¹, —OS(O)₂R¹¹, —CH₂OC(O)R¹¹, —OC(O)R¹¹, —OC₁-C₆-alkyl-C(O)OR¹¹, —OC₁-C₆-alkyl-OR¹¹, —SC₁-C₆-alkyl-C(O)OR¹¹, —C(O)OR¹¹, or —C₂-C₆-alkenyl-C(═O)R¹¹,
C₁-C₆-alkyl or C₁-C₆— which may each optionally be substituted with one or more substituents independently selected from R¹³
aryl, aryloxy, aroyl, aryl-C₁-C₆-alkoxy, aryl-C₁-C₆-alkyl, heteroaryl,
of which each of the cyclic moieties optionally may be substituted with one or more substituents independently selected from R¹⁴.
   Embodiment 42. A pharmaceutical composition according to embodiment 41 wherein R⁷, R⁸, R⁹ and R¹⁰ are independently selected from
hydrogen, halogen, —OR¹¹, —OC₁-C₆-alkyl-C(O)OR¹¹, or —C(O)OR¹¹,
C₁-C₆-alkyl which may each optionally be substituted with one or more substituents independently selected from R¹³
aryl, aryloxy, aryl-C₁-C₆-alkoxy,
of which each of the cyclic moieties optionally may be substituted with one or more substituents independently selected from R¹⁴.
   Embodiment 43. A pharmaceutical composition according to embodiment 42 wherein R⁷, R⁸, R⁹ and R¹⁰ are independently selected from
hydrogen, halogen, —OR¹¹, —OC₁-C₆-alkyl-C(O)OR¹¹, or —C(O)OR¹¹,
C₁-C₆-alkyl which may each optionally be substituted with one or more substituents independently selected from R¹³
ArG1, ArG1oxy, ArG1-C₁-C₆-alkoxy,
   of which each of the cyclic moieties optionally may be substituted with one or more substituents independently selected from R¹⁴.
   Embodiment 44. A pharmaceutical composition according to embodiment 43 wherein R⁷, R⁸, R⁹ and R¹⁰ are independently selected from
hydrogen, halogen, —OR¹¹, —OC₁-C₆-alkyl-C(O)OR¹¹, or —C(O)OR¹¹,
C₁-C₆-alkyl which may optionally be substituted with one or more substituents independently selected from R¹³
phenyl, phenyloxy, phenyl-C₁-C₆-alkoxy, wherein each of the cyclic moieties optionally may be substituted with one or more substituents independently selected from R¹⁴.
   Embodiment 45. A pharmaceutical composition according to any one of the embodiments 2 to 44 wherein R¹¹ and R¹² are independently selected from hydrogen, C₁-C₂₀-alkyl, aryl or aryl-C₁-C₆-alkyl, wherein the alkyl groups may optionally be substituted with one or more substituents independently selected from R¹⁵, and the aryl groups may optionally be substituted one or more substituents independently selected from R¹⁶; R¹¹ and R¹² when attached to the same nitrogen atom may form a 3 to 8 membered heterocyclic ring with the said nitrogen atom, the heterocyclic ring optionally containing one or two further heteroatoms selected from nitrogen, oxygen and sulphur, and optionally containing one or two double bonds.
   Embodiment 46. A pharmaceutical composition according to embodiment 45 wherein R¹¹ and R¹² are independently selected from hydrogen, C₁-C₂₀-alkyl, aryl or aryl-C₁-C₆-alkyl, wherein the alkyl groups may optionally be substituted with one or more substituents independently selected from R¹⁵, and the aryl groups may optionally be substituted one or more substituents independently selected from R¹⁶.
   Embodiment 47. A pharmaceutical composition according to embodiment 46 wherein R¹¹ and R¹² are independently selected from phenyl or phenyl-C₁-C₆-alkyl.
   Embodiment 48. A pharmaceutical composition according to embodiment 46 wherein one or both of R¹¹ and R¹² are methyl.
   Embodiment 49. A pharmaceutical composition according to any one of the embodiments 2 to 48 wherein R¹³ is independently selected from halogen, CF₃, OR¹¹ or NR¹¹R¹².
   Embodiment 50. A pharmaceutical composition according to embodiment 49 wherein R¹³ is independently selected from halogen or OR¹¹.
   Embodiment 51. A pharmaceutical composition according to embodiment 50 wherein R¹³ is OR¹¹.
   Embodiment 52. A pharmaceutical composition according to any one of the embodiments 2 to 51 wherein R¹⁴ is independently selected from halogen, —C(O)OR¹¹, —CN, —CF₃, —OR¹¹, S(O)₂R¹¹, and C₁-C₆-alkyl.
   Embodiment 53. A pharmaceutical composition according to embodiment 52 wherein R¹⁴ is independently selected from halogen, —C(O)OR¹¹, or —OR¹¹.
   Embodiment 54. A pharmaceutical composition according to any one of the embodiments 2 to 53 wherein R¹⁵ is independently selected from halogen, —CN, —CF₃, —C(O)OC₁-C₆-alkyl, and —COOH.
   Embodiment 55. A pharmaceutical composition according to embodiment 54 wherein R¹⁵ is independently selected from halogen or —C(O)OC₁-C₆-alkyl.
   Embodiment 56. A pharmaceutical composition according to any one of the embodiments 2 to 55 wherein R¹⁶ is independently selected from halogen, —C(O)OC₁-C₆-alkyl, —COOH, —NO₂, —OC₁-C₆-alkyl, —NH₂, C(═O) or C₁-C₆-alkyl.
   Embodiment 57. A pharmaceutical composition according to embodiment 56 wherein R¹⁶ is independently selected from halogen, —C(O)OC₁-C₆-alkyl, —COOH, —NO₂, or C₁-C₆-alkyl.
   Embodiment 58. A pharmaceutical composition according to embodiment 1 wherein the ligand for the HisB10 anion site is

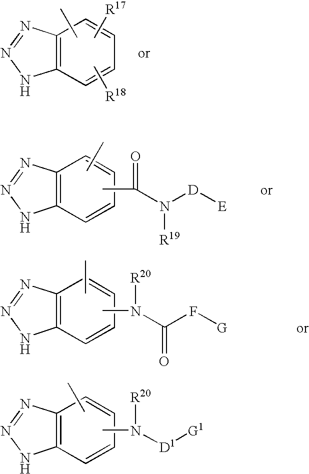

wherein
R¹⁹ is hydrogen or C₁-C₆-alkyl,
R²⁰ is hydrogen or C₁-C₆-alkyl,
D, D¹ and F are a valence bond, C₁-C₆-alkylene or C₁-C₆-alkenylene optionally substituted with one or more substituents independently selected from R⁷²,
R⁷² is independently selected from hydroxy, C₁-C₆-alkyl, or aryl,
E is C₁-C₆-alkyl, aryl or heteroaryl, wherein the aryl or heteroaryl is optionally substituted with up to three substituents R²¹, R²² and R²³,
G and G¹ are C₁-C₆-alkyl, aryl or heteroaryl, wherein the aryl or heteroaryl is optionally substituted with up to three substituents R²⁴, R²⁵ and R²⁶,
R¹⁷, R¹⁸, R²¹, R²², R²³, R²⁴, R²⁵ and R²⁶ are independently selected from
hydrogen, halogen, —CN, —CH₂CN, —CHF₂, —CF₃, —OCF₃, —OCHF₂, —OCH₂CF₃, —OCF₂CHF₂, —S(O)₂CF₃, —SCF₃, —NO₂, ═O, —OR²⁷, —NR²⁷R²⁸, —SR²⁷, —NR²⁷S(O)₂R²⁸, —S(O)₂NR²⁷R²⁸, —S(O)NR²⁷R²⁸, —S(O)R²⁷, —S(O)₂R²⁷, —C(O)NR²⁷R²⁸, —OC(O)NR²⁷R²⁸, —NR²⁷C(O)R²⁸, —NR²⁷C(O)OR²⁸, —CH₂C(O)NR²⁷R²⁸, —OCH₂C(O)NR²⁷R²⁸, —CH₂OR²⁷, —CH₂NR²⁷R²⁸, —OC(O)R²⁷, —OC₁-C₆-alkyl-C(O)OR²⁷, —SC₁-C₆-alkyl-C(O)OR²⁷, —C₂-C₆-alkenyl-C(═O)OR²⁷, —NR²⁷—C(═O)—C₁-C₆-alkyl-C(═O)OR²⁷, —NR²⁷—C(═O)—C₁-C₆-alkenyl-C(═O)OR²⁷, —C(═O)NR²⁷—C₁-C₆-alkyl-C(═O)OR²⁷, —C₁-C₆-alkyl-C(═O)OR²⁷, or —C(O)OR²⁷,
C₁-C₆-alkyl, C₂-C₆-alkenyl or C₂-C₆-alkynyl,
which may optionally be substituted with one or more substituents independently selected from R²⁹,
aryl, aryloxy, aryloxycarbonyl, aroyl, aryl-C₁-C₆-alkoxy, aryl-C₁-C₆-alkyl, aryl-C₂-C₆-alkenyl, aryl-C₂-C₆-alkynyl, heteroaryl, heteroaryl-C₁-C₆-alkyl, heteroaryl-C₂-C₆-alkenyl or heteroaryl-C₂-C₆-alkynyl,
of which the cyclic moieties optionally may be substituted with one or more substituents selected from R³⁰,
   R²⁷ and R²⁸ are independently selected from hydrogen, C₁-C₆-alkyl, aryl-C₁-C₆-alkyl or aryl, or R²⁷ and R²⁸ when attached to the same nitrogen atom together with the said nitrogen atom may form a 3 to 8 membered heterocyclic ring optionally containing one or two further heteroatoms selected from nitrogen, oxygen and sulphur, and optionally containing one or two double bonds,
   R²⁹ is independently selected from halogen, —CN, —CF₃, —OCF₃, —OR²⁷, and —NR²⁷R²⁸,
   R³⁰ is independently selected from halogen, —C(O)OR²⁷, —CN, —CF₃, —OCF₃, —NO₂, —OR²⁷, —NR²⁷R²⁸ and C₁-C₆-alkyl, or any enantiomer, diastereomer, including a racemic mixture, tautomer as well as a salt thereof with a pharmaceutically acceptable acid or base.
   Embodiment 59. A pharmaceutical composition according to embodiment 58 wherein D is a valence bond.
   Embodiment 60. A pharmaceutical composition according to embodiment 58 wherein D is C₁-C₆-alkylene optionally substituted with one or more hydroxy, C₁-C₆-alkyl, or aryl.
   Embodiment 61. A pharmaceutical composition according to any one of the embodiments 58 to 60 wherein E is aryl or heteroaryl, wherein the aryl or heteroaryl is optionally substituted with up to three substituents independently selected from R²¹, R²² and R²³.
   Embodiment 62. A pharmaceutical composition according to embodiment 61 wherein E is aryl optionally substituted with up to three substituents independently selected from R²¹, R²² and R²³.
   Embodiment 63. A pharmaceutical composition according to embodiment 62 wherein E is selected from ArG1 and optionally substituted with up to three substituents independently selected from R²¹, R²² and R²³.
   Embodiment 64. A pharmaceutical composition according to embodiment 63 wherein E is phenyl optionally substituted with up to three substituents independently selected from R²¹, R²² and R²³.
   Embodiment 65. A pharmaceutical composition according to embodiment 64 wherein the ligand for the His^{B10} anion site is

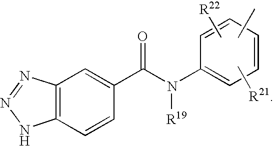

Embodiment 66. A pharmaceutical composition according to any one of the embodiments 58 to 65 wherein R²¹, R²² and R²³ are independently selected from
hydrogen, halogen, —CHF₂, —CF₃, —OCF₃, —OCHF₂, —OCH₂CF₃, —OCF₂CHF₂, —SCF₃, —NO₂, —R²⁷, —NR²⁷R²⁸, —SR²⁷, —C(O)NR²⁷R²⁸, —OC(O)NR²⁷R²⁸, —NR²⁷C(O)R²⁸, —NR²⁷C(O)OR²⁸, —CH₂C(O)NR²⁷R²⁸, —OCH₂C(O)NR²⁷R²⁸, —CH₂OR²⁷, —CH₂NR²⁷R²⁸, —OC(O)R²⁷, —OC₁-C₆-alkyl-C(O)OR²⁷, —SC₁-C₆-alkyl-C(O)OR²⁷, —C₂-C₆-alkenyl-C(═O)OR²⁷, —NR²⁷—C(═O)—C₁-C₆-alkyl-C(═O)OR²⁷, —NR²⁷—C(═O)—C₁-C₆-alkenyl-C(═O)OR²⁷—, —C(═O)NR²⁷—C₁-C₆-alkyl-C(═O)OR²⁷, —C₁-C₆-alkyl-C(═O)OR²⁷, or —C(O)OR²⁷,
C₁-C₆-alkyl, C₂-C₆-alkenyl or C₂-C₆-alkynyl,
which may optionally be substituted with one or more substituents independently selected from R²⁹
aryl, aryloxy, aryloxycarbonyl, aroyl, aryl-C₁-C₆-alkoxy, aryl-C₁-C₆-alkyl, aryl-C₂-C₆-alkenyl, aryl-C₂-C₆-alkynyl, heteroaryl, heteroaryl-C₁-C₆-alkyl, heteroaryl-C₂-C₆-alkenyl or heteroaryl-C₂-C₆-alkynyl,
of which the cyclic moieties optionally may be substituted with one or more substituents selected from R³⁰.
   Embodiment 67. A pharmaceutical composition according to embodiment 66 wherein R²¹, R²² and R²³ are independently selected from
hydrogen, halogen, —OCF₃, —OR²⁷, —NR²⁷R²⁸, —SR²⁷, —NR²⁷C(O)R²⁸, —NR²⁷C(O)OR²⁸, —OC(O)R²⁷, —OC₁-C₆-alkyl-C(O)OR²⁷, —SC₁-C₆-alkyl-C(O)OR²⁷, —C₂-C₆-alkenyl-C(═O)OR²⁷, —C(═O)NR²⁷—C₁-C₆-alkyl-C(═O)OR²⁷, —C₁-C₆-alkyl-C(═O)OR²⁷, or —C(O)OR
C₁-C₆-alkyl optionally substituted with one or more substituents independently selected from R²⁹
aryl, aryloxy, aroyl, aryl-C₁-C₆-alkoxy, aryl-C₁-C₆-alkyl, heteroaryl, heteroaryl-C₁-C₆-alkyl,
of which the cyclic moieties optionally may be substituted with one or more substituents selected from R³⁰.
   Embodiment 68. A pharmaceutical composition according to embodiment 67 wherein R²¹, R²² and R²³ are independently selected from
hydrogen, halogen, —OCF₃, —OR²⁷, —NR²⁷R²⁸, —SR²⁷, —NR²⁷C(O)R²⁸, —NR²⁷C(O)R²⁸, —OC(O)R²⁷, —OC₁-C₆-alkyl-C(O)OR²⁷, —SC₁-C₆-alkyl-C(O)OR²⁷, —C₂-C₆-alkenyl-C(═O)OR²⁷, —C(═O)NR²⁷—C₁-C₆-alkyl-C(═O)OR²⁷, —C₁-C₆-alkyl-C(═O)OR²⁷, or —C(O)OR²⁷,
methyl, ethyl propyl optionally substituted with one or more substituents independently selected from R²⁹
aryl, aryloxy, aroyl, aryl-C₁-C₆-alkoxy, aryl-C₁-C₆-alkyl, heteroaryl, heteroaryl-C₁-C₆-alkyl
of which the cyclic moieties optionally may be substituted with one or more substituents selected from R³⁰.
   Embodiment 69. A pharmaceutical composition according to embodiment 68 wherein R²¹, R²² and R²³ are independently selected from
hydrogen, halogen, —OCF₃, —R²⁷, —NR²⁷R²⁸, —SR²⁷, —NR²⁷C(O)R²⁸, —NR²⁷C(O)OR²⁸, —OC(O)R²⁷, —OC₁-C₆-alkyl-C(O)OR²⁷, —SC₁-C₆-alkyl-C(O)OR²⁷, —C₂-C₆-alkenyl-C(═O)OR²⁷, —C(═O)NR²⁷—C₁-C₆-alkyl-C(═O)OR²⁷, —C₁-C₆-alkyl-C(═O)OR²⁷, or —C(O)OR²⁷,
methyl, ethyl propyl optionally substituted with one or more substituents independently selected from R²⁹
ArG1, ArG1-O—, ArG1-C(O)—, ArG1-C₁-C₆-alkoxy, ArG1-C₁-C₆-alkyl, Het3, Het3-C₁-C₆-alkyl
   of which the cyclic moieties optionally may be substituted with one or more substituents selected from R³⁰.
   Embodiment 70. A pharmaceutical composition according to embodiment 69 wherein R²¹, R²² and R²³ are independently selected from
hydrogen, halogen, —OCF₃, —OR²⁷, —NR²⁷R²⁸, —SR²⁷, —NR²⁷C(O)R²⁸, —NR²⁷C(O)OR²⁸, —OC(O)R²⁷, —OC₁-C₆-alkyl-C(O)OR²⁷, —SC₁-C₆-alkyl-C(O)OR²⁷, —C₂-C₆-alkenyl-C(═O)OR²⁷, —C(═O)NR²⁷—C₁-C₆-alkyl-C(═O)OR²⁷, —C₁-C₆-alkyl-C(═O)OR²⁷, or —C(O)OR
C₁-C₆-alkyl optionally substituted with one or more substituents independently selected from R²⁹
phenyl, phenyloxy, phenyl-C₁-C₆-alkoxy, phenyl-C₁-C₆-alkyl,
   of which the cyclic moieties optionally may be substituted with one or more substituents selected from R³⁰.
   Embodiment 71. A pharmaceutical composition according to any one of the embodiments 58 to 70 wherein R¹⁹ is hydrogen or methyl.
   Embodiment 72. A pharmaceutical composition according to embodiment 71 wherein R¹⁹ is hydrogen.
   Embodiment 73. A pharmaceutical composition according to any one of the embodiments 58 to 72 wherein R²⁷ is Hydrogen, C₁-C₆-alkyl or aryl.
   Embodiment 74. A pharmaceutical composition according to embodiment 73 wherein R²⁷ is hydrogen or C₁-C₆-alkyl.
   Embodiment 75. A pharmaceutical composition according to any one of the embodiments 58 to 74 wherein R²⁸ is hydrogen or C₁-C₆-alkyl.
   Embodiment 76. A pharmaceutical composition according to embodiment 58 wherein F is a valence bond.
   Embodiment 77. A pharmaceutical composition according to embodiment 58 wherein F is C₁-C₆-alkylene optionally substituted with one or more hydroxy, C₁-C₆-alkyl, or aryl.
   Embodiment 78. A pharmaceutical composition according to any one of the embodiments 58 or 76 to 77 wherein G is C₁-C₆-alkyl or aryl, wherein the aryl is optionally substituted with up to three substituents R²⁴, R²⁵ and R²⁶.
   Embodiment 79. A pharmaceutical composition according to any one of the embodiments 58 or 76 to 77 wherein G is C₁-C₆-alkyl or ArG1, wherein the aryl is optionally substituted with up to three substituents R²⁴, R²⁵ and R²⁶.
   Embodiment 80. A pharmaceutical composition according to embodiment 78 wherein G is C₁-C₆-alkyl.
   Embodiment 81. A pharmaceutical composition according to embodiment 80 wherein G is phenyl optionally substituted with up to three substituents R²⁴, R²⁵ and R²⁶.
   Embodiment 82. A pharmaceutical composition according to any one of the embodiments 58 to 81 wherein R²⁴, R²⁵ and R²⁶ are independently selected from
hydrogen, halogen, —CHF₂, —CF₃, —OCF₃, —OCHF₂, —OCH₂CF₃, —OCF₂CHF₂, —SCF₃, —N₂, —OR²⁷, —NR²⁷R²⁸, —SR²⁷, —C(O)NR²⁷R²⁸, —OC(O)NR²⁷R²⁸, —NR²⁷C(O)R²⁸, —NR²⁷C(O)OR²⁸, —CH₂C(O)NR²⁷R²⁸, —OCH₂C(O)NR²⁷R²⁸, —CH₂OR²⁷, —CH₂NR²⁷R²⁸, —OC(O)R²⁷, —OC₁-C₆-alkyl-C(O)OR²⁷, —SC₁-C₆-alkyl-C(O)OR²⁷, —C₂-C₆-alkenyl-C(═O)OR²⁷, —NR²⁷—C(═O)—C₁-C₆-alkyl-C(═O)OR²⁷, —NR²⁷—C(═O)—C₁-C₆-alkenyl-C(═O)OR²⁷—, —C(═O)NR²⁷—C₁-C₆-alkyl-C(═O)OR²⁷, —C₁-C₆-alkyl-C(═O)OR²⁷, or —C(O)OR²⁷,
C₁-C₆-alkyl, C₂-C₆-alkenyl or C₂-C₆-alkynyl,
which may optionally be substituted with one or more substituents independently selected from R²⁹
aryl, aryloxy, aryloxycarbonyl, aroyl, aryl-C₁-C₆-alkoxy, aryl-C₁-C₆-alkyl, aryl-C₂-C₆-alkenyl, aryl-C₂-C₆-alkynyl, heteroaryl, heteroaryl-C₁-C₆-alkyl, heteroaryl-C₂-C₆-alkenyl or heteroaryl-C₂-C₆-alkynyl,
of which the cyclic moieties optionally may be substituted with one or more substituents selected from R³⁰.
   Embodiment 83. A pharmaceutical composition according to embodiment 82 wherein R²⁴, R²⁵ and R²⁶ are independently selected from
hydrogen, halogen, —OCF₃, —OR²⁷, —NR²⁷R²⁸, —SR²⁷, —NR²⁷C(O)R²⁸, —NR²⁷C(O)OR²⁸, —OC(O)R²⁷, —OC₁-C₆-alkyl-C(O)OR²⁷, —SC₁-C₆-alkyl-C(O)OR²⁷, —C₂-C₆-alkenyl-C(═O)OR²⁷, —C(═O)NR²⁷—C₁-C₆-alkyl-C(═O)OR²⁷, —C₁-C₆-alkyl-C(═O)OR²⁷, or —C(O)OR²⁷,
C₁-C₆-alkyl, C₂-C₆-alkenyl or C₂-C₆-alkynyl,
which may optionally be substituted with one or more substituents independently selected from R²⁹
aryl, aryloxy, aryloxycarbonyl, aroyl, aryl-C₁-C₆-alkoxy, aryl-C₁-C₆-alkyl, aryl-C₂-C₆-alkenyl, aryl-C₂-C₆-alkynyl, heteroaryl, heteroaryl-C₁-C₆-alkyl, heteroaryl-C₂-C₆-alkenyl or heteroaryl-C₂-C₆-alkynyl,
of which the cyclic moieties optionally may be substituted with one or more substituents selected from R³⁰.
   Embodiment 84. A pharmaceutical composition according to embodiment 83 wherein R²⁴, R²⁵ and R²⁶ are independently selected from
hydrogen, halogen, —OCF₃, —OR²⁷, —NR²⁷R²⁸, —SR²⁷, —NR²⁷C(O)R²⁸, —NR²⁷C(O)OR²⁸, —OC(O)R²⁷, —OC₁-C₆-alkyl-C(O)OR²⁷, —SC₁-C₆-alkyl-C(O)OR²⁷, —C₂-C₆-alkenyl-C(═O)OR²⁷, —C(═O)NR²⁷—C₁-C₆-alkyl-C(═O)OR²⁷, —C₁-C₆-alkyl-C(═O)OR²⁷, or —C(O)OR²⁷,
C₁-C₆-alkyl optionally substituted with one or more substituents independently selected from R²⁹
aryl, aryloxy, aroyl, aryl-C₁-C₆-alkoxy, aryl-C₁-C₆-alkyl, heteroaryl, heteroaryl-C₁-C₆-alkyl,
of which the cyclic moieties optionally may be substituted with one or more substituents selected from R³⁰.
   Embodiment 85. A pharmaceutical composition according to embodiment 84 wherein R²¹, R²² and R²³ are independently selected from
hydrogen, halogen, —OCF₃, —OR²⁷, —NR²⁷R²⁸, —SR²⁷, —NR²⁷C(O)R²⁸, —NR²⁷C(O)OR²⁸, —OC(O)R²⁷, —OC₁-C₆-alkyl-C(O)OR²⁷, —SC₁-C₆-alkyl-C(O)OR²⁷, —C₂-C₆-alkenyl-C(═O)OR²⁷, —C(═O)NR²⁷—C₁-C₆-alkyl-C(═O)OR²⁷, —C₁-C₆-alkyl-C(═O)OR²⁷, or —C(O)OR²⁷,
methyl, ethyl propyl optionally substituted with one or more substituents independently selected from R²⁹
ArG1, ArG1-O—, ArG1-C(O)—, ArG1-C₁-C₆-alkoxy, ArG1-C₁-C₆-alkyl, Het3, Het3-C₁-C₆-alkyl
   of which the cyclic moieties optionally may be substituted with one or more substituents selected from R³⁰.
   Embodiment 86. A pharmaceutical composition according to embodiment 85 wherein R²¹, R²² and R²³ are independently selected from
hydrogen, halogen, —OCF₃, —OR²⁷, —NR²⁷R²⁸, —SR²⁷, —NR²⁷C(O)R²⁸—NR²⁷C(O)OR²⁸, —OC(O)R²⁷, —OC₁-C₆-alkyl-C(O)OR²⁷, —SC₁-C₆-alkyl-C(O)OR²⁷, —C₂-C₆-alkenyl-C(═O)OR²⁷, —C(═O)NR²⁷—C₁-C₆-alkyl-C(═O)OR²⁷, —C₁-C₆-alkyl-C(═O)OR²⁷, or —C(O)OR²⁷,
methyl, ethyl propyl optionally substituted with one or more substituents independently selected from R²⁹
ArG1, ArG1-O—, ArG1-C(O)—, ArG1-C₁-C₆-alkoxy, ArG1-C₁-C₆-alkyl, Het3, Het3-C₁-C₆-alkyl
of which the cyclic moieties optionally may be substituted with one or more substituents selected from R³⁰.
   Embodiment 87. A pharmaceutical composition according to embodiment 86 wherein R²¹, R²² and R²³ are independently selected from
hydrogen, halogen, —OCF₃, —OR²⁷, —NR²⁷R²⁸, —SR²⁷, —NR²⁷C(O)R²⁸, —NR²⁷C(O)OR²⁸, (—C(O)R²⁷, —OC₁-C₆-alkyl-C(O)OR²⁷, —SC₁-C₆-alkyl-C(O)OR²⁷, —C₂-C₆-alkenyl-C(═O)OR²⁷, —C(═O)NR²⁷—C₁-C₆-alkyl-C(═O)OR²⁷, —C₁-C₆-alkyl-C(═O)OR²⁷, or —C(O)OR²⁷,
methyl, ethyl propyl optionally substituted with one or more substituents independently selected from R²⁹
ArG1, ArG1-O—, ArG1-C₁-C₆-alkoxy, ArG1-C₁-C₆-alkyl,
   of which the cyclic moieties optionally may be substituted with one or more substituents selected from R³⁰.
   Embodiment 88. A pharmaceutical composition according to any one of the embodiments 58 or 76 to 87 wherein R²⁰ is hydrogen or methyl.
   Embodiment 89. A pharmaceutical composition according to embodiment 88 wherein R²⁰ is hydrogen.
   Embodiment 90. A pharmaceutical composition according to any one of the embodiments 58 or 76 to 89 wherein R²⁷ is hydrogen, C₁-C₆-alkyl or aryl.
   Embodiment 91. A pharmaceutical composition according to embodiment 90 wherein R²⁷ is hydrogen or C₁-C₆-alkyl or ArG1.
   Embodiment 92. A pharmaceutical composition according to embodiment 91 wherein R²⁷ is hydrogen or C₁-C₆-alkyl.
   Embodiment 93. A pharmaceutical composition according to any one of the embodiments 58 or 76 to 91 wherein R²⁸ is hydrogen or C₁-C₆-alkyl.
   Embodiment 94. A pharmaceutical composition according to embodiment 58 wherein R¹⁷ and R¹⁸ are independently selected from
hydrogen, halogen, —CN, —CF₃, —OCF₃, —NO₂, —OR²⁷, —NR²⁷R²⁸, —SR²⁷, —S(O)R²⁷, —S(O)₂R²⁷, —C(O)NR²⁷R²⁸, —CH₂OR²⁷, —OC(O)R²⁷, —OC₁-C₆-alkyl-C(O)OR²⁷, —SC₁-C₆-alkyl-C(O)OR²⁷, or —C(O)OR²⁷,
C₁-C₆-alkyl, C₂-C₆-alkenyl or C₂-C₆-alkynyl, optionally substituted with one or more substituents independently selected from R²⁹
aryl, aryloxy, aroyl, aryl-C₁-C₆-alkoxy, aryl-C₁-C₆-alkyl, heteroaryl, heteroaryl-C₁-C₆-alkyl,
of which the cyclic moieties optionally may be substituted with one or more substituents selected from R³⁰.
   Embodiment 95. A pharmaceutical composition according to embodiment 94 wherein R¹⁷ and R¹⁸ are independently selected from
hydrogen, halogen, —CN, —CF₃, —NO₂, —OR²⁷, —NR²⁷R²⁸, or —C(O)OR²⁷,
C₁-C₆-alkyl optionally substituted with one or more substituents independently selected from R²⁹
aryl, aryloxy, aroyl, aryl-C₁-C₆-alkoxy, aryl-C₁-C₆-alkyl, heteroaryl, heteroaryl-C₁-C₆-alkyl,
of which the cyclic moieties optionally may be substituted with one or more substituents selected from R³⁰.
   Embodiment 96. A pharmaceutical composition according to embodiment 95 wherein R¹⁷ and R¹⁸ are independently selected from
hydrogen, halogen, —CN, —CF₃, —NO₂, —OR²⁷, —NR²⁷R²⁸, or —C(O)OR²⁷
methyl, ethyl propyl optionally substituted with one or more substituents independently selected from R²⁹
aryl, aryloxy, aroyl, aryl-C₁-C₆-alkoxy, aryl-C₁-C₆-alkyl, heteroaryl, heteroaryl-C₁-C₆-alkyl
of which the cyclic moieties optionally may be substituted with one or more substituents selected from R³⁰.
   Embodiment 97. A pharmaceutical composition according to embodiment 96 wherein R¹⁷ and R¹⁸ are independently selected from
hydrogen, halogen, —CN, —CF₃, —NO₂, —OR²⁷, —NR²⁷R²⁸, or —C(O)OR²⁷
methyl, ethyl propyl optionally substituted with one or more substituents independently selected from R²⁹
ArG1, ArG1-O—, ArG1-C(O)—, ArG1-C₁-C₆-alkoxy, ArG1-C₁-C₆-alkyl, Het3, Het3-C₁-C₆-alkyl
   of which the cyclic moieties optionally may be substituted with one or more substituents selected from R³⁹.
   Embodiment 98. A pharmaceutical composition according to embodiment 97 wherein R¹⁷ and R¹⁸ are independently selected from
hydrogen, halogen, —CN, —CF₃, —NO₂, —OR²⁷, —NR²⁷R²⁸, or —C(O)OR²⁷
C₁-C₆-alkyl optionally substituted with one or more substituents independently selected from R²⁹
phenyl, phenyloxy, phenyl-C₁-C₆-alkoxy, phenyl-C₁-C₆-alkyl,
   of which the cyclic moieties optionally may be substituted with one or more substituents selected from R³⁰.
   Embodiment 99. A pharmaceutical composition according to any one of the embodiments 58 to 98 wherein R²⁷ is hydrogen or C₁-C₆-alkyl.
   Embodiment 100. A pharmaceutical composition according to embodiment 99 wherein R²⁷ is hydrogen, methyl or ethyl.
   Embodiment 101. A pharmaceutical composition according to any one of the embodiments 58 to 100 wherein R²⁸ is hydrogen or C₁-C₆-alkyl.
   Embodiment 102. A pharmaceutical composition according to embodiment 101 wherein R²⁸ is hydrogen, methyl or ethyl.
   Embodiment 103. A pharmaceutical composition according to any one of the embodiments 58 to 102 wherein R⁷² is —OH or phenyl.
   Embodiment 104. A pharmaceutical composition according to embodiment 58 wherein the ligand for the His^{B10} anion site is

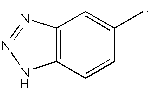

Embodiment 105. A pharmaceutical composition according to embodiment 1 wherein the ligand for the His^{B10} anion site is of the form H-I-J
wherein H is

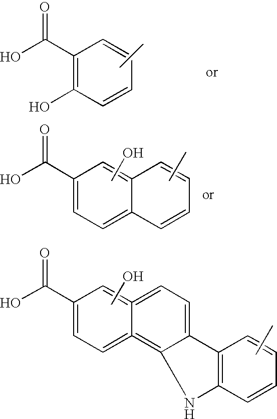

wherein the phenyl, naphthalene or benzocarbazole rings are optionally substituted with one or more substituents independently selected from R³¹
I is selected from
a valence bond,
—CH₂N(R³²)— or —SO₂N(R³³)—,

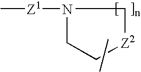

wherein Z¹ is S(O)₂ or CH₂, Z² is —NH—, —O— or —S—, and n is 1 or 2, J is
C₁-C₆-alkyl, C₂-C₆-alkenyl or C₂-C₆-alkynyl, which may each optionally be substituted with one or more substituents selected from R³⁴,
Aryl, aryloxy, aryl-oxycarbonyl-, aroyl, aryl-C₁-C₆-alkoxy-, aryl-C₁-C₆-alkyl-, aryl-C₂-C₆-alkenyl-, aryl-C₂-C₆-alkynyl-, heteroaryl, heteroaryl-C₁-C₆-alkyl-, heteroaryl-C₂-C₆-alkenyl- or heteroaryl-C₂-C₆-alkynyl-, wherein the cyclic moieties are optionally substituted with one or more substituents selected from R³⁷,
hydrogen,
   R³¹ is independently selected from hydrogen, halogen, —CN, —CH₂CN, —CHF₂, —CF₃, —OCF₃, —OCHF₂, —OCH₂CF₃, —OCF₂CHF₂, —S(O)₂CF₃, —SCF₃, —NO₂, —OR³⁵, —C(O)R³⁵, —NR³⁵R³⁵, —SR³⁵, —NR³S(O)₂R³⁶, —S(O)₂NR³⁵R³⁶, —S(O)NR³⁵R³⁶, —S(O)R³⁵, —S(O)₂R³⁵, —C(O)NR³⁵R³⁶, —OC(O)NR³⁵R³⁶, —NR³⁵C(O)R³⁶, —CH₂C(O)NR³⁵R³⁶, —OCH₂C(O)NR³⁵R³⁶, —CH₂OR³⁵, —CH₂NR³⁵R³⁶, —OC(O)R³⁵, —OC₁-C₆-alkyl-C(O)OR³⁵, —SC₁-C₆-alkyl-C(O)OR³⁵—C₂-C₆-alkenyl-C(═O)OR³⁵, —NR³⁵—C(═O)—C₁₋₆-alkyl-C(═O)OR³⁵, —NR³⁵—C(═O)—C₁-C₆-alkenyl-C(═O)OR³⁵, C₁-C₆-alkyl, C₁-C₆-alkanoyl or —C(O)OR³⁵,
   R³² and R³³ are independently selected from hydrogen, C₁-C₆-alkyl or C₁-C₆-alkanoyl,
   R³⁴ is independently selected from halogen, —CN, —CF₃, —OCF₃, —OR³⁵, and —NR³⁵R³⁶,
   R³⁵ and R³⁶ are independently selected from hydrogen, C₁-C₆-alkyl, aryl-C₁-C₆-alkyl or aryl, or R³⁵ and R³⁶ when attached to the same nitrogen atom together with the said nitrogen atom may form a 3 to 8 membered heterocyclic ring optionally containing one or two further heteroatoms selected from nitrogen, oxygen and sulphur, and optionally containing one or two double bonds,
   R³⁷ is independently selected from halogen, —C(O)OR³⁵, —C(O)H, —CN, —CF₃, —OCF₃, —NO₂, —OR³⁵, —NR³⁵R³⁶, C₁-C₆-alkyl or C₁-C₆-alkanoyl,
   or any enantiomer, diastereomer, including a racemic mixture, tautomer as well as a salt thereof with a pharmaceutically acceptable acid or base.
   Embodiment 106. A pharmaceutical composition according to embodiment 105 wherein the ligand for the His^{B10} anion site is of the form H-I-J, wherein H is

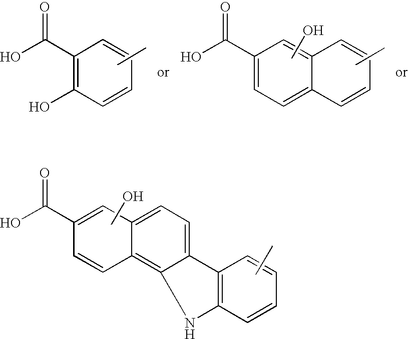

wherein the phenyl, naphthalene or benzocarbazole rings are optionally substituted with one or more substituents independently selected from R³¹,
I is selected from
a valence bond,
—CH₂N(R³²)— or —SO₂N(R³³)—,

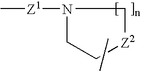

wherein Z¹ is S(O)₂ or CH₂, Z² is N, —O— or —S—, and n is 1 or 2,

### J is

C₁-C₆-alkyl, C₂-C₆-alkenyl or C₂-C₆-alkynyl, which may each optionally be substituted with one or more substituents selected from R³⁴,
Aryl, aryloxy, aryl-oxycarbonyl-, aroyl, aryl-C₁-C₆-alkoxy-, aryl-C₁-C₆-alkyl-, aryl-C₂-C₆-alkenyl-, aryl-C₂-C₆-alkynyl-, heteroaryl, heteroaryl-C₁-C₆-alkyl-, heteroaryl-C₂-C₆-alkenyl- or heteroaryl-C₂-C₆-alkynyl-, wherein the cyclic moieties are optionally substituted with one or more substituents selected from R³⁷,
hydrogen,
   R³¹ is independently selected from hydrogen, halogen, —CN, —CH₂CN, —CHF₂, —CF₃, —OCF₃, —OCHF₂, —OCH₂CF₃, —OCF₂CHF₂, —S(O)₂CF₃, —SCF₃, —NO₂, —OR³⁵, —C(O)R³⁵, —NR³⁵R³⁶, —SR³⁵, —NR³⁵S(O)₂R³⁶, —S(O)₂NR³⁵R³⁶, —S(O)NR³⁵R³⁶, —S(O)R³⁵, —S(O)₂R³⁵, —C(O)NR³⁵R³⁶, —OC(O)NR³⁵R³⁶, —NR³⁵C(O)R³⁶, —CH₂C(O)NR³⁵R³⁶, —OCH₂C(O)NR³⁵R³⁶, —CH₂OR³⁵, —CH₂NR³⁵R³⁶, —OC(O)R³⁵, —OC₁-C₆-alkyl-C(O)OR³⁵, —SC₁-C₆-alkyl-C(O)OR³⁵—C₂-C₆-alkenyl-C(═O)OR³⁵, —NR³⁵—C(═O)—C₁-C₆-alkyl-C(═O)OR³⁵, —NR³⁵—C(═O)—C₁-C₆-alkenyl-C(═O)OR³⁵—, C₁-C₆-alkyl, C₁-C₆-alkanoyl or —C(O)OR³⁵,
   R³² and R³³ are independently selected from hydrogen, C₁-C₆-alkyl or C₁-C₆-alkanoyl,
   R³⁴ is independently selected from halogen, —CN, —CF₃, —OCF₃, —OR³⁵, and —NR³⁵R³⁶,
   R³⁵ and R³⁶ are independently selected from hydrogen, C₁-C₆-alkyl, aryl-C₁-C₆-alkyl or aryl, or R³⁵ and R³⁶ when attached to the same nitrogen atom together with the said nitrogen atom may form a 3 to 8 membered heterocyclic ring optionally containing one or two further heteroatoms selected from nitrogen, oxygen and sulphur, and optionally containing one or two double bonds,
   R³⁷ is independently selected from halogen, —C(O)OR³⁵, —C(O)H, —CN, —CF₃, —OCF₃, —NO₂, —OR³⁵, —NR³⁵R³⁶, C₁-C₆-alkyl or C₁-C₆-alkanoyl,
   or any enantiomer, diastereomer, including a racemic mixture, tautomer as well as a salt thereof with a pharmaceutically acceptable acid or base,
   With the proviso that R³¹ and J cannot both be hydrogen.
   Embodiment 107. A pharmaceutical composition according to any one of the embodiments 105 or 106 wherein H is

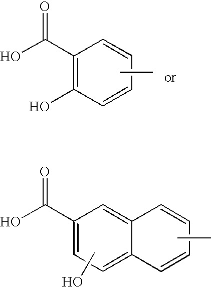

Embodiment 108. A pharmaceutical composition according to embodiment 107 wherein H is

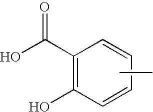

Embodiment 109. A pharmaceutical composition according to embodiment 107 wherein H is

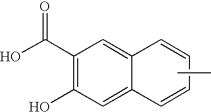

Embodiment 110. A pharmaceutical composition according to any one of the embodiments 105 to 109 wherein I is a valence bond, —CH₂N(R³²)—, or —SO₂N(R³³)—.
Embodiment 111. A pharmaceutical composition according to embodiment 110 wherein I is a valence bond.
Embodiment 112. A pharmaceutical composition according to any one of the embodiments 105 to 111 wherein J is
hydrogen,
C₁-C₆-alkyl, C₂-C₆-alkenyl or C₂-C₆-alkynyl, which may optionally be substituted with one or more substituents selected from halogen, —CN, —CF₃, —OCF₃, —OR³⁵, and —NR³⁵R³⁶,
aryl, or heteroaryl, wherein the cyclic moieties are optionally substituted with one or more substituents independently selected from R³⁷.
   Embodiment 113. A pharmaceutical composition according to embodiment 112 wherein J is
hydrogen,
aryl or heteroaryl, wherein the cyclic moieties are optionally substituted with one or more substituents independently selected from R³⁷.
   Embodiment 114. A pharmaceutical composition according to embodiment 112 wherein J is
hydrogen,
ArG1 or Het3, wherein the cyclic moieties are optionally substituted with one or more substituents independently selected from R³⁷.
   Embodiment 115. A pharmaceutical composition according to embodiment 114 wherein J is
hydrogen,
phenyl or naphthyl optionally substituted with one or more substituents independently selected from R³⁷.
   Embodiment 116. A pharmaceutical composition according to embodiment 115 wherein J is hydrogen.
   Embodiment 117. A pharmaceutical composition according to any one of the embodiments 105 to 116 wherein R³² and R³³ are independently selected from hydrogen or C₁-C₆-alkyl.
   Embodiment 118. A pharmaceutical composition according to any one of the embodiments 105 to 117 wherein R³⁴ is hydrogen, halogen, —CN, —CF₃, —OCF₃, —SCF₃, —NO₂, —OR³⁵, —C(O)R³⁵, —NR³⁵R³⁶, —SR³⁵, —C(O)NR³⁵R³⁶, —OC(O)NR³⁵R³⁶, —NR³⁵C(O)R³⁶, —OC(O)R³⁵, —OC₁-C₆-alkyl-C(O)OR³⁵, —SC₁-C₆-alkyl-C(O)OR³⁵ or —C(O)OR³⁵.
   Embodiment 119. A pharmaceutical composition according to embodiment 118 wherein R³⁴ is hydrogen, halogen, —CF₃, —NO₂, —OR³⁵, —NR³⁵R³⁶, —SR³⁵, —NR³⁵C(O)R³⁶, or —C(O)OR³⁵.
   Embodiment 120. A pharmaceutical composition according to embodiment 119 wherein R³⁴ is hydrogen, halogen, —CF₃, —NO₂, —OR³⁵, —NR³⁵R³⁶, or —NR³⁵C(O)R³⁶.
   Embodiment 121. A pharmaceutical composition according to embodiment 120 wherein R³⁴ is hydrogen, halogen, or —OR³⁵.
   Embodiment 122. A pharmaceutical composition according to any one of the embodiments 105 to 121 wherein R³⁵ and R³⁶ are independently selected from hydrogen, C₁-C₆-alkyl, or aryl.
   Embodiment 123. A pharmaceutical composition according to embodiment 122 wherein R³⁵ and R³⁶ are independently selected from hydrogen or C₁-C₆-alkyl.
   Embodiment 124. A pharmaceutical composition according to any one of the embodiments 105 to 123 wherein R³⁷ is halogen, —C(O)OR³⁵, —CN, —CF₃, —OR³⁵, —NR³⁵R³⁶, C₁-C₆-alkyl or C₁-C₆-alkanoyl.
   Embodiment 125. A pharmaceutical composition according to embodiment 124 wherein R³⁷ is halogen, —C(O)OR³⁵, —OR³⁵, —NR³⁵R³⁶, C₁-C₆-alkyl or C₁-C₆-alkanoyl.
   Embodiment 126. A pharmaceutical composition according to embodiment 125 wherein R³⁷ is halogen, —C(O)OR³⁵ or —OR³⁵.
   Embodiment 127. A pharmaceutical composition according to embodiment 1 wherein the ligand for the His^{B10} anion site is

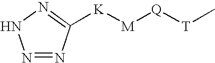

wherein K is a valence bond, C₁-C₆-alkylene, —NH—C(═O)—U—, —C₁-C₆-alkyl-S—, —C₁-C₆-alkyl-O—, —C(═O)—, or —C(═O)—NH—, wherein any C₁-C₆-alkyl moiety is optionally substituted with R³⁸,
U is a valence bond, C₁-C₆-alkenylene, —C₁-C₆-alkyl-O— or C₁-C₆-alkylene wherein any C₁-C₆-alkyl moiety is optionally substituted with C₁-C₆-alkyl,
R³⁸ is C₁-C₆-alkyl, aryl, wherein the alkyl or aryl moieties are optionally substituted with one or more substituents independently selected from R³⁹,
R³⁹ is independently selected from halogen, cyano, nitro, amino,
M is a valence bond, arylene or heteroarylene, wherein the aryl or heteroaryl moieties are optionally substituted with one or more substituents independently selected from R⁴⁰,
R⁴⁰ is selected from
hydrogen, halogen, —CN, —CH₂CN, —CHF₂, —CF₃, —OCF₃, —OCHF₂, —OCH₂CF₃, —OCF₂CHF₂, —S(O)₂CF₃, —OS(O)₂CF₃, —SCF₃, —NO₂, —OR⁴¹, —NR⁴¹R⁴², —SR⁴¹, —NR⁴¹S(O)₂R⁴², —S(O)₂NR⁴¹R⁴², —S(O)NR⁴¹R⁴², —S(O)R⁴¹, —S(O)₂R⁴¹, —OS(O)₂R⁴¹, —C(O)NR⁴¹R⁴², —OC(O)NR⁴¹R⁴², —NR⁴¹C(O)R⁴², —CH₂C(O)NR⁴¹R⁴², —OC₁-C₆-alkyl-C(O)NR⁴¹R⁴², —CH₂OR⁴¹, —CH₂OC(O)R⁴¹, —CH₂NR⁴¹R⁴², —OC(O)R⁴¹, —OC₁-C₆-alkyl-C(O)OR⁴¹, —OC₁-C₆-alkyl-OR⁴¹, —S—C₁-C₆-alkyl-C(O)OR⁴¹, —C₂-C₆-alkenyl-C(═O)OR⁴¹, —NR⁴¹—C(═O)—C₁-C₆-alkyl-C(═O)OR⁴¹, —NR⁴¹—C(═O)—C₁-C₆-alkenyl-C(═O)OR⁴¹, —C(O)OR⁴¹, —C₂-C₆-alkenyl-C(═O)R⁴¹, ═O, —NH—C(═O)—O—C₁-C₆-alkyl, or —NH—C(═O)—C(═O)—O—C₁-C₆-alkyl,
C₁-C₆-alkyl, C₂-C₆-alkenyl or C₂-C₆-alkynyl, which may each optionally be substituted with one or more substituents selected from R⁴³,
aryl, aryloxy, aryloxycarbonyl, aroyl, arylsulfanyl, aryl-C₁-C₆-alkoxy, aryl-C₁-C₆-alkyl, aryl-C₂-C₆-alkenyl, aroyl-C₂-C₆-alkenyl, aryl-C₂-C₆-alkynyl, heteroaryl, heteroaryl-C₁-C₆-alkyl, heteroaryl-C₂-C₆-alkenyl or heteroaryl-C₂-C₆-alkynyl, wherein the cyclic moieties optionally may be substituted with one or more substituents selected from R⁴⁴,
   R⁴¹ and R⁴² are independently selected from hydrogen, —OH, C₁-C₆-alkyl, C₁-C₆-alkenyl, aryl-C₁-C₆-alkyl or aryl, wherein the alkyl moieties may optionally be substituted with one or more substituents independently selected from R⁴⁵, and the aryl moieties may optionally be substituted with one or more substituents independently selected from R⁴⁶; R⁴¹ and R⁴² when attached to the same nitrogen atom may form a 3 to 8 membered heterocyclic ring with the said nitrogen atom, the heterocyclic ring optionally containing one or two further heteroatoms selected from nitrogen, oxygen and sulphur, and optionally containing one or two double bonds,
   R⁴³ is independently selected from halogen, —CN, —CF₃, —OCF₃, —OR⁴¹, and —NR⁴¹R⁴²
   R⁴⁴ is independently selected from halogen, —C(O)OR⁴¹, —CH₂C(O)OR⁴¹, —CH₂OR⁴¹, —CN, —CF₃, —OCF₃, —NO₂, —OR⁴¹, —NR⁴¹R⁴² and C₁-C₆-alkyl,
   R⁴⁵ is independently selected from halogen, —CN, —CF₃, —OCF₃, —O—C₁-C₆-alkyl, —C(O)—O—C₁-C₆-alkyl, —COOH and —NH₂,
   R⁴⁶ is independently selected from halogen, —C(O)OC₁-C₆-alkyl, —COOH, —CN, —CF₃, —OCF₃, —NO₂, —OH, —OC₁-C₆-alkyl, —NH₂, C(═O) or C₁-C₆-alkyl,
   Q is a valence bond, C₁-C₆-alkylene, —C₁-C₆-alkyl-O—, —C₁-C₆-alkyl-NH—, —NH—C₁-C₆-alkyl, —NH—C(═O)—, —C(═O)—NH—, —O—C₁-C₆-alkyl, —C(═O)—, or —C₁-C₆-alkyl-C(═O)—N(R⁴⁷)— wherein the alkyl moieties are optionally substituted with one or more substituents independently selected from R⁴⁸,
   R⁴⁷ and R⁴⁸ are independently selected from hydrogen, C₁-C₆-alkyl, aryl optionally substituted with one or more R⁴⁹,
   R⁴⁹ is independently selected from halogen and —COOH,

### T is

hydrogen,
C₁-C₆-alkyl, C₂-C₆-alkenyl, C₂-C₆-alkynyl, C₁-C₆-alkyloxy-carbonyl, wherein the alkyl, alkenyl and alkynyl moieties are optionally substituted with one or more substituents independently selected from R⁵⁰,
aryl, aryloxy, aryloxy-carbonyl, aryl-C₁-C₆-alkyl, aroyl, aryl-C₁-C₆-alkoxy, aryl-C₂-C₆-alkenyl, aryl-C₂-C₆-alkyny-, heteroaryl, heteroaryl-C₁-C₆-alkyl, heteroaryl-C₂-C₆-alkenyl, heteroaryl-C₂-C₆-alkynyl,
wherein any alkyl, alkenyl, alkynyl, aryl and heteroaryl moiety is optionally substituted with one or more substituents independently selected from R⁵⁰,
   R⁵⁰ is C₁-C₆-alkyl, C₁-C₆-alkoxy, aryl, aryloxy, aryl-C₁-C₆-alkoxy, —C(═O)—NH—C₁-C₆-alkyl-aryl, —C(═O)—NR^{50A}—C₁-C₆-alkyl, —C(═O)—NH—(CH₂CH₂O)ₘC₁-C₆-alkyl-COOH, heteroaryl, heteroaryl-C₁-C₆-alkoxy, —C₁-C₆-alkyl-COOH, —O—C₁-C₆-alkyl-COOH, —S(O)₂R⁵¹, —C₂-C₆-alkenyl-COOH, —OR⁵¹, —NO₂, halogen, —COOH, —CF₃, —CN, ═O, —N(R⁵¹R⁵²), wherein m is 1, 2, 3 or 4, and wherein the aryl or heteroaryl moieties are optionally substituted with one or more R⁵³, and the alkyl moieties are optionally substituted with one or more R^{50B}.
   Embodiment R^{50A} and R^{50B} are independently selected from —C(O)OC₁-C₆-alkyl, —COOH, —C₁-C₆-alkyl-C(O)OC₁-C₆-alkyl, —C₁-C₆-alkyl-COOH, or C₁-C₆-alkyl,
   R⁵¹ and R⁵² are independently selected from hydrogen and C₁-C₆-alkyl,
   R⁵³ is independently selected from C₁-C₆-alkyl, C₁-C₆-alkoxy, —C₁-C₆-alkyl-COOH, —C₂-C₆-alkenyl-COOH, —OR⁵¹, —NO₂, halogen, —COOH, —CF₃, —CN, or —N(R⁵¹R⁵²),
   or any enantiomer, diastereomer, including a racemic mixture, tautomer as well as a salt thereof with a pharmaceutically acceptable acid or base.
   Embodiment 128. A pharmaceutical composition according to embodiment 127 wherein K is a valence bond, C₁-C₆-alkylene, —NH—C(═O)—U—, —C₁-C₆-alkyl-S—, —C₁-C₆-alkyl-O—, or —C(═O)—, wherein any C₁-C₆-alkyl moiety is optionally substituted with R³⁸.
   Embodiment 129. A pharmaceutical composition according to embodiment 128 wherein K is a valence bond, C₁-C₆-alkylene, —NH—C(═O)—U—, —C₁-C₆-alkyl-S—, or —C₁-C₆-alkyl-0, wherein any C₁-C₆-alkyl moiety is optionally substituted with R³⁸.
   Embodiment 130. A pharmaceutical composition according to embodiment 129 wherein K is a valence bond, C₁-C₆-alkylene, or —NH—C(═O)—U, wherein any C₁-C₆-alkyl moiety is optionally substituted with R³⁸.
   Embodiment 131. A pharmaceutical composition according to embodiment 130 wherein K is a valence bond or C₁-C₆-alkylene, wherein any C₁-C₆-alkyl moiety is optionally substituted with R³⁸.
   Embodiment 132. A pharmaceutical composition according to embodiment 130 wherein K is a valence bond or —NH—C(═O)—U.
   Embodiment 133. A pharmaceutical composition according to embodiment 131 wherein K is a valence bond.
   Embodiment 134. A pharmaceutical composition according to any one of the embodiments 127 to 133 wherein U is a valence bond or —C₁-C₆-alkyl-O—.
   Embodiment 135. A pharmaceutical composition according to embodiment 134 wherein U is a valence bond.
   Embodiment 136. A pharmaceutical composition according to any one of the embodiments 127 to 135 wherein M is arylene or heteroarylene, wherein the arylene or heteroarylene moieties are optionally substituted with one or more substituents independently selected from R⁴⁰.
   Embodiment 137. A pharmaceutical composition according to embodiment 136 wherein M is ArG1 or Het1, wherein the arylene or heteroarylene moieties are optionally substituted with one or more substituents independently selected from R⁴⁰.
   Embodiment 138. A pharmaceutical composition according to embodiment 137 wherein M is ArG1 or Het2, wherein the arylene or heteroarylene moieties are optionally substituted with one or more substituents independently selected from R⁴⁰.
   Embodiment 139. A pharmaceutical composition according to embodiment 138 wherein M is ArG1 or Het3, wherein the arylene or heteroarylene moieties are optionally substituted with one or more substituents independently selected from R⁴⁰.
   Embodiment 140. A pharmaceutical composition according to embodiment 139 wherein M is phenylene optionally substituted with one or more substituents independently selected from R⁴⁰.
   Embodiment 141. A pharmaceutical composition according to embodiment 139 wherein M is indolylene optionally substituted with one or more substituents independently selected from R⁴⁰.
   Embodiment 142. A pharmaceutical composition according to embodiment 141 wherein M is

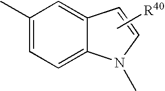

143. A pharmaceutical composition according to embodiment 139 wherein M is carbazolylene optionally substituted with one or more substituents independently selected from R⁴⁰.
Embodiment 144. A pharmaceutical composition according to embodiment 143 wherein M is

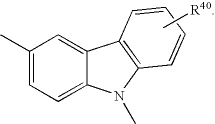

Embodiment 145. A pharmaceutical composition according to any one of the embodiments 127 to 144 wherein R⁴⁰ is selected from
hydrogen, halogen, —CN, —CF₃, —OCF₃, —NO₂, —OR⁴¹, —NR⁴¹R⁴², —SR⁴¹, —S(O)₂R⁴¹, —NR⁴¹C(O)R⁴², —OC₁-C₆-alkyl-C(O)NR⁴¹R⁴², —C₂-C₆-alkenyl-C(═O)OR⁴¹, —C(O)OR⁴¹, ═O, —NH—C(═O)—O—C₁-C₆-alkyl, or —NH—C(═O)—C(═O)—O—C₁-C₆-alkyl,
C₁-C₆-alkyl or C₂-C₆— alkenyl which may each optionally be substituted with one or more substituents independently selected from R⁴³,
aryl, aryloxy, aryl-C₁-C₆-alkoxy, aryl-C₁-C₆-alkyl, aryl-C₂-C₆-alkenyl, heteroaryl, heteroaryl-C₁-C₆-alkyl, or heteroaryl-C₂-C₆-alkenyl, wherein the cyclic moieties optionally may be substituted with one or more substituents selected from R⁴⁴.
   Embodiment 146. A pharmaceutical composition according to embodiment 145 wherein R⁴⁰ is selected from
hydrogen, halogen, —CN, —CF₃, —OCF₃, —NO₂, —OR⁴¹, —NR⁴¹R⁴², —SR⁴¹, —S(O)₂R⁴¹, —NR⁴¹C(O)R⁴², —OC₁-C₆-alkyl-C(O)NR⁴¹R⁴², —C₂-C₆-alkenyl-C(═O)OR⁴¹, —C(O)OR⁴¹, ═O, —NH—C(═O)—O—C₁-C₆-alkyl, or —NH—C(═O)—C(═O)—O—C₁-C₆-alkyl,
C₁-C₆-alkyl or C₂-C₆— alkenyl which may each optionally be substituted with one or more substituents independently selected from R⁴³,
ArG1, ArG1-O—, ArG1-C₁-C₆-alkoxy, ArG1-C₁-C₆-alkyl, ArG1-C₂-C₆-alkenyl, Het3, Het3-C₁-C₆-alkyl, or Het3-C₂-C₆-alkenyl, wherein the cyclic moieties optionally may be substituted with one or more substituents selected from R⁴⁴.
   Embodiment 147. A pharmaceutical composition according to embodiment 146 wherein R⁴⁰ is selected from
hydrogen, halogen, —CF₃, —NO₂, —OR⁴¹, —NR⁴¹R⁴², —C(O)OR⁴¹, ═O, or —NR⁴¹C(O)R⁴²,
C₁-C₆-alkyl,
ArG1.
   Embodiment 148. A pharmaceutical composition according to embodiment 147 wherein R⁴⁰ is hydrogen.
   Embodiment 149. A pharmaceutical composition according to embodiment 147 wherein R⁴⁰ is selected from
halogen, —NO₂, —OR⁴¹, —NR⁴¹R⁴², —C(O)OR⁴¹, or —NR⁴¹C(O)R⁴²,
methyl,
phenyl.
   Embodiment 150. A pharmaceutical composition according to any one of the embodiments 127 to 149 wherein R⁴¹ and R⁴² are independently selected from hydrogen, C₁-C₆-alkyl, or aryl, wherein the aryl moieties may optionally be substituted with halogen or —COOH.
   Embodiment 151. A pharmaceutical composition according to embodiment 150 wherein R⁴¹ and R⁴² are independently selected from hydrogen, methyl, ethyl, or phenyl, wherein the phenyl moieties may optionally be substituted with halogen or —COOH.
   Embodiment 152. A pharmaceutical composition according to any one of the embodiments 127 to 151 wherein Q is a valence bond, C₁-C₆-alkylene, —C₁-C₆-alkyl-O—, —C₁-C₆-alkyl-NH—, —NH—C₁-C₆-alkyl, —NH—C(═O)—, —C(═O)—NH—, —O—C₁-C₆-alkyl, —C(═O)—, or —C₁-C₆-alkyl-C(═O)—N(R⁴⁷)— wherein the alkyl moieties are optionally substituted with one or more substituents independently selected from R⁴⁸.
   Embodiment 153. A pharmaceutical composition according to embodiment 152 wherein Q is a valence bond, —CH₂—, —CH₂—CH₂—, —CH₂—O—, —CH₂—CH₂—O—, —CH₂—NH—, —CH₂—CH₂—NH—, —NH—CH₂—, —NH—CH₂—CH₂—, —NH—C(═O)—, —C(═O)—NH—, —O—CH₂—, —O—CH₂—CH₂—, or —C(═O)—.
   Embodiment 154. A pharmaceutical composition according to any one of the embodiments 127 to 153 wherein R⁴⁷ and R⁴⁸ are independently selected from hydrogen, methyl and phenyl.
   Embodiment 155. A pharmaceutical composition according to any one of the embodiments 127 to 154 wherein T is
hydrogen,
C₁-C₆-alkyl optionally substituted with one or more substituents independently selected from R⁵⁰,
aryl, aryl-C₁-C₆-alkyl, heteroaryl, wherein the alkyl, aryl and heteroaryl moieties are optionally substituted with one or more substituents independently selected from R⁵⁰.
   Embodiment 156. A pharmaceutical composition according to embodiment 155 wherein T is
hydrogen,
C₁-C₆-alkyl optionally substituted with one or more substituents independently selected from R⁵⁰,
ArG1, ArG1-C₁-C₆-alkyl, Het3, wherein the alkyl, aryl and heteroaryl moieties are optionally substituted with one or more substituents independently selected from R⁵⁰.
   Embodiment 157. A pharmaceutical composition according to embodiment 156 wherein T is
hydrogen,
C₁-C₆-alkyl, optionally substituted with one or more substituents independently selected from R⁵⁰,
phenyl, phenyl-C₁-C₆-alkyl, wherein the alkyl and phenyl moieties are optionally substituted with one or more substituents independently selected from R⁵⁰.
   Embodiment 158. A pharmaceutical composition according to embodiment 157 wherein T is phenyl substituted with R⁵⁰.
   Embodiment 159. A pharmaceutical composition according to any one of the embodiments 127 to 158 wherein R⁵⁰ is C₁-C₆-alkyl, C₁-C₆-alkoxy, aryl, aryloxy, aryl-C₁-C₆-alkoxy, —C(═O)—NH—C₁-C₆-alkyl-aryl, —C(═O)—NR^{50A}—C₁-C₆-alkyl, —C(═O)—NH—(CH₂CH₂O)ₘC₁-C₆-alkyl-COOH, heteroaryl, —C₁-C₆-alkyl-COOH, —O—C₁-C₆-alkyl-COOH, —S(O)₂R⁵¹, —C₂-C₆-alkenyl-COOH, —OR⁵¹, —NO₂, halogen, —COOH, —CF₃, —CN, ═O, —N(R⁵¹R⁵²), wherein the aryl or heteroaryl moieties are optionally substituted with one or more R⁵³.
   Embodiment 160. A pharmaceutical composition according to embodiment 159 wherein R⁵⁰ is C₁-C₆-alkyl, C₁-C₆-alkoxy, aryl, aryloxy, —C(═O)—NR^{50A}—C₁-C₆-alkyl, —C(═O)—NH—(CH₂CH₂O)ₘC₁-C₆-alkyl-COOH, aryl-C₁-C₆-alkoxy, —OR⁵¹, —NO₂, halogen, —COOH, —CF₃, wherein any aryl moiety is optionally substituted with one or more R⁵³.
   Embodiment 161. A pharmaceutical composition according to embodiment 160 wherein R⁵⁰ is C₁-C₆-alkyl, aryloxy, —C(═O)—NR^{50A}—C₁-C₆-alkyl, —C(═O)—NH—(CH₂CH₂O)ₘC₁-C₆-alkyl-COOH, aryl-C₁-C₆-alkoxy, —OR⁵¹, halogen, —COOH, —CF₃, wherein any aryl moiety is optionally substituted with one or more R⁵³.
   Embodiment 162. A pharmaceutical composition according to embodiment 161 wherein R⁵⁰ is C₁-C₆-alkyl, ArG1-O—, —C(═O)—NR^{50A}—C₁-C₆-alkyl, —C(═O)—NH—(CH₂CH₂O)ₘC₁-C₆-alkyl-COOH, ArG1-C₁-C₆-alkoxy, —OR⁵¹, halogen, —COOH, —CF₃, wherein any aryl moiety is optionally substituted with one or more R⁵³.
   Embodiment 163. A pharmaceutical composition according to embodiment 162 wherein R⁵⁰ is —C(═O)—NR^{50A}CH₂, —C(═O)—NH—(CH₂CH₂O)₂CH₂I—COOH, or —C(═O)—NR^{50A}CH₂CH₂.
   Embodiment 164. A pharmaceutical composition according to embodiment 162 wherein R⁵⁰ is phenyl, methyl or ethyl.
   Embodiment 165. A pharmaceutical composition according to embodiment 164 wherein R⁵⁰ is methyl or ethyl.
   Embodiment 166. A pharmaceutical composition according to any one of the embodiments 127 to 165 wherein m is 1 or 2.
   Embodiment 167. A pharmaceutical composition according to any one of the embodiments 127 to 166 wherein R⁵¹ is methyl.
   Embodiment 168. A pharmaceutical composition according to any one of the embodiments 127 to 167 wherein R⁵³ is C₁-C₆-alkyl, C₁-C₆-alkoxy, —OR⁵¹, halogen, or —CF₃.
   Embodiment 169. A pharmaceutical composition according to any one of the embodiments 127 to 168 wherein R^{50A} is —C(O)OCH₃, —C(O)OCH₂CH₃—COOH, —CH₂C(O)OCH₃, —CH₂C(O)OCH₂CH₃, —CH₂CH₂C(O)OCH₃, —CH₂CH₂C(O)OCH₂CH₃, —CH₂COOH, methyl, or ethyl.
   Embodiment 170. A pharmaceutical composition according to any one of the embodiments 127 to 169 wherein R^{50B} is —C(O)OCH₃, —C(O)OCH₂CH₃—COOH, —CH₂C(O)OCH₃, —CH₂C(O)OCH₂CH₃, —CH₂CH₂C(O)OCH₃, —CH₂CH₂C(O)OCH₂CH₃, —CH₂COOH, methyl, or ethyl.
   Embodiment 171. A pharmaceutical composition according to embodiment 1 wherein the ligand for the His^{B10} anion site is

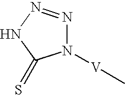

wherein V is C₁-C₆-alkyl, aryl, heteroaryl, aryl-C₁₋₆-alkyl- or aryl-C₂₋₆-alkenyl-, wherein the alkyl or alkenyl is optionally substituted with one or more substituents independently selected from R⁵⁴, and the aryl or heteroaryl is optionally substituted with one or more substituents independently selected from R⁵⁵,
R⁵⁴ is independently selected from halogen, —CN, —CF₃, —OCF₃, aryl, —COOH and —NH₂,
R⁵⁵ is independently selected from
hydrogen, halogen, —CN, —CH₂CN, —CHF₂, —CF₃, —OCF₃, —OCHF₂, —OCH₂CF₃, —OCF₂CHF₂, —S(O)₂CF₃, —OS(O)₂CF₃, —SCF₃, —NO₂, —OR⁵⁶, —NR⁵⁶R⁵⁷, —SR⁵⁶, —NR⁶S(O)₂R⁵⁷, —S(O)₂NR⁵⁶R⁵⁷, —S(O)NR⁵⁶R⁵⁷, —S(O)R⁵⁶, —S(O)₂R⁵⁶, —OS(O)₂R⁵⁶, —C(O)NR⁵⁶R⁵⁷, —OC(O)NR⁵⁶R⁵⁷, —NR⁵⁶C(O)R⁵⁷, —CH₂C(O)NR⁵⁶R⁵⁷, —OC₁-C₆-alkyl-C(O)NR⁵⁶R⁵⁷, —CH₂OR⁵⁶, —CH₂OC(O)R⁵⁶, —CH₂NR⁵⁶R⁵⁷, —OC(O)R⁵⁶—OC₁-C₈-alkyl-C(O)OR⁵⁶, —OC₁-C₆-alkyl-OR⁵⁶, —SC₁-C₆-alkyl-C(O)OR⁵⁶, —C₂-C₆-alkenyl-C(═O)OR⁵⁶, —NR⁵⁶—C(═O)—C₁-C₆-alkyl-C(═O)OR⁵⁶, —NR⁵⁶—C(═O)—C₁-C₆-alkenyl-C(═O)OR⁵⁶, —C(O)OR⁵⁶, or —C₂-C₆-alkenyl-C(═O)R⁵⁶,
C₁-C₆-alkyl, C₂-C₆-alkenyl or C₂-C₆-alkynyl,
which may optionally be substituted with one or more substituents selected from R⁵⁸,
aryl, aryloxy, aryloxycarbonyl, aroyl, arylsulfanyl, aryl-C₁-C₆-alkoxy, aryl-C₁-C₆-alkyl, aryl-C₂-C₆-alkenyl, aroyl-C₂-C₆-alkenyl, aryl-C₂-C₆-alkynyl, heteroaryl, heteroaryl-C₁-C₆-alkyl, heteroaryl-C₂-C₆-alkenyl or heteroaryl-C₂-C₆-alkynyl,
of which the cyclic moieties optionally may be substituted with one or more substituents selected from R⁵⁹,
   R⁵⁶ and R⁵⁷ are independently selected from hydrogen, OH, CF₃, C₁-C₁₂-alkyl, aryl-C₁-C₆-alkyl, —C(═O)—C₁-C₆-alkyl or aryl, wherein the alkyl groups may optionally be substituted with one or more substituents independently selected from R⁶⁰, and the aryl groups may optionally be substituted with one or more substituents independently selected from R⁶¹; R⁵⁶ and R⁵⁷ when attached to the same nitrogen atom may form a 3 to 8 membered heterocyclic ring with the said nitrogen atom, the heterocyclic ring optionally containing one or two further heteroatoms selected from nitrogen, oxygen and sulphur, and optionally containing one or two double bonds,
   R⁵⁸ is independently selected from halogen, —CN, —CF₃, —OCF₃, —OR⁵⁶, and —NR⁵⁶R⁵⁷,
   R⁵⁹ is independently selected from halogen, —C(O)OR⁵⁶, —CH₂C(O)OR⁵⁶, —CH₂OR⁵⁶, —CN, —CF₃, —OCF₃, —NO₂, —OR⁵⁶, —NR⁵⁶R⁵⁷ and C₁-C₆-alkyl,
   R⁶⁰ is independently selected from halogen, —CN, —CF₃, —OCF₃, —OC₁-C₆-alkyl, —C(O)OC₁-C₆-alkyl, —C(═O)—R⁶², —COOH and —NH₂,
   R⁶¹ is independently selected from halogen, —C(O)OC₁-C₆-alkyl, —COOH, —CN, —CF₃, —OCF₃, —NO₂, —OH, —OC₁-C₆-alkyl, —NH₂, C(═O) or C₁-C₆-alkyl,
   R⁶² is C₁-C₆-alkyl, aryl optionally substituted with one or more substituents independently selected from halogen, or heteroaryl optionally substituted with one or more C₁-C₆-alkyl independently,
   or any enantiomer, diastereomer, including a racemic mixture, tautomer as well as a salt thereof with a pharmaceutically acceptable acid or base.
   Embodiment 172. A pharmaceutical composition according to embodiment 171 wherein V is aryl, heteroaryl, or aryl-C₁₋₆-alkyl-, wherein the alkyl is optionally substituted with one or more substituents independently selected R⁵⁴, and the aryl or heteroaryl is optionally substituted with one or more substituents independently selected from R⁵⁵.
   Embodiment 173. A pharmaceutical composition according to embodiment 172 wherein V is aryl, Het1, or aryl-C₁₋₆-alkyl-, wherein the alkyl is optionally substituted with one or more substituents independently selected from R⁵⁴, and the aryl or heteroaryl moiety is optionally substituted with one or more substituents independently selected from R⁵⁵.
   Embodiment 174. A pharmaceutical composition according to embodiment 173 wherein V is aryl, Het2, or aryl-C₁₋₆-alkyl-, wherein the alkyl is optionally substituted with one or more substituents independently selected from R⁵⁴, and the aryl or heteroaryl moiety is optionally substituted with one or more substituents independently selected from R⁵⁵.
   Embodiment 175. A pharmaceutical composition according to embodiment 174 wherein V is aryl, Het3, or aryl-C₁₋₆-alkyl-, wherein the alkyl is optionally substituted with one or more substituents independently selected from R⁵⁴, and the aryl or heteroaryl moiety is optionally substituted with one or more substituents independently selected from R⁵⁵.
   Embodiment 176. A pharmaceutical composition according to embodiment 175 wherein V is aryl optionally substituted with one or more substituents independently selected from R⁵⁵.
   Embodiment 177. A pharmaceutical composition according to embodiment 176 wherein V is ArG1 optionally substituted with one or more substituents independently selected from R⁵⁵.
   Embodiment 178. A pharmaceutical composition according to embodiment 177 wherein V is phenyl, naphthyl or anthranyl optionally substituted with one or more substituents independently selected from R⁵⁵.
   Embodiment 179. A pharmaceutical composition according to embodiment 178 wherein V is phenyl optionally substituted with one or more substituents independently selected from R⁵⁵.
   Embodiment 180. A pharmaceutical composition according to any one of the embodiments 171 to 179 wherein R⁵⁵ is independently selected from
halogen, C₁-C₆-alkyl, —CN, —OCF₃, —CF₃, —NO₂, —OR⁵⁶, —NR⁵⁶R⁵⁷, —NR⁵⁶C(O)R⁵⁷—SR⁵⁶, —OC₁-C₈-alkyl-C(O)OR⁵⁶, or —C(O)OR⁵⁶, C₁-C₆-alkyl optionally substituted with one or more substituents independently selected from R⁵⁸
aryl, aryl-C₁-C₆-alkyl, heteroaryl, or heteroaryl-C₁-C₆-alkyl of which the cyclic moieties optionally may be substituted with one or more substituents independently selected from R⁵⁹.
   Embodiment 181. A pharmaceutical composition according to embodiment 180 wherein R⁵⁵ is independently selected from
halogen, C₁-C₆-alkyl, —CN, —OCF₃, —CF₃, —NO₂, —OR⁵⁶, —NR⁵⁶R⁵⁷, —NR⁵⁶C(O)R⁵⁷—SR⁵⁶, —OC₁-C₈-alkyl-C(O)OR⁵⁶, or —C(O)OR⁵⁶
C₁-C₆-alkyl optionally substituted with one or more substituents independently selected from R⁵⁸
ArG1, ArG1-C₁-C₆-alkyl, Het3, or Het3-C₁-C₆-alkyl of which the cyclic moieties optionally may be substituted with one or more substituents independently selected from R⁵⁹.
   Embodiment 182. A pharmaceutical composition according to embodiment 181 wherein R⁵⁵ is independently selected from halogen, —OR⁵⁶, —NR⁵⁶R⁵⁷, —C(O)OR⁵⁶, —OC₁-C₈-alkyl-C(O)OR⁵⁶, —NR⁵⁶C(O)R⁵⁷ or C₁-C₆-alkyl.
   Embodiment 183. A pharmaceutical composition according to embodiment 182 wherein R⁵⁵ is independently selected from halogen, —OR⁶⁶, —NR⁵⁶R⁵⁷, —C(O)OR⁵⁶, —OC₁-C₈-alkyl-C(O)OR⁵⁶, —NR⁵⁶C(O)R⁵⁷, methyl or ethyl.
   Embodiment 184. A pharmaceutical composition according to any one of the embodiments 171 to 183 wherein R⁵⁶ and R⁵⁷ are independently selected from hydrogen, CF₃, C₁-C₁₂-alkyl, or —C(═O)—C₁-C₆-alkyl; R⁵⁶ and R⁵⁷ when attached to the same nitrogen atom may form a 3 to 8 membered heterocyclic ring with the said nitrogen atom.
   Embodiment 185. A pharmaceutical composition according to embodiment 184 wherein R⁵⁶ and R⁵⁷ are independently selected from hydrogen or C₁-C₁₂-alkyl, R⁵⁶ and R⁵⁷ when attached to the same nitrogen atom may form a 3 to 8 membered heterocyclic ring with the said nitrogen atom.
   Embodiment 186. A pharmaceutical composition according to embodiment 185 wherein R⁵⁶ and R⁵⁷ are independently selected from hydrogen or methyl, ethyl, propyl butyl, R⁵⁶ and R⁵⁷ when attached to the same nitrogen atom may form a 3 to 8 membered heterocyclic ring with the said nitrogen atom.
   Embodiment 187. A pharmaceutical composition according to embodiment 1 wherein the ligand for the His^{B30} anion site is

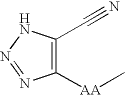

wherein AA is C₁-C₆-alkyl, aryl, heteroaryl, aryl-C₁₋₆-alkyl- or aryl-C₂₋₆-alkenyl-, wherein the alkyl or alkenyl is optionally substituted with one or more substituents independently selected from R⁶³, and the aryl or heteroaryl is optionally substituted with one or more substituents independently selected from R⁶⁴,
R⁶³ is independently selected from halogen, —CN, —CF₃, —OCF₃, aryl, —COOH and —NH₂,
R⁶⁴ is independently selected from
hydrogen, halogen, —CN, —CH₂CN, —CHF₂, —CF₃, —OCF₃, —OCHF₂, —OCH₂CF₃, —OCF₂CHF₂, —S(O)₂CF₃, —OS(O)₂CF₃, —SCF₃, —NO₂, —OR⁶⁵, —NR⁶⁵R⁶⁶, —SR⁶⁵, —NR⁶⁵S(O)₂R⁶, —S(O)₂NR⁶⁵R⁶⁶, —S(O)NR⁶⁵R⁶⁶, —S(O)R⁶⁵, —S(O)R⁶⁵, —OS(O)₂R⁶⁵, —C(O)NR⁶⁵R⁶⁶, —OC(O)NR⁶⁵R⁶⁶, —NR⁶⁵C(O)R⁶⁶, —CH₂C(O)NR⁶⁵R⁶⁶, —OC₁-C₆-alkyl-C(O)NR⁶⁵R⁶⁶, —CH₂OR⁶⁵, —CH₂OC(O)R⁶⁵, —CH₂NR⁶⁵R⁶⁶, —OC(O)R⁶⁵, —OC₁-C₆-alkyl-C(O)OR⁶⁵, —OC₁-C₆-alkyl-OR⁶⁵, —SC₁-C₆-alkyl-C(O)OR⁶⁵, —C₂-C₆-alkenyl-C(═O)OR⁶⁵, —NR⁶⁵—C(═O)—C₁-C₆-alkyl-C(═O)OR⁶⁵, —NR⁶⁵—C(═O)—C₁-C₆-alkenyl-C(═O)OR⁶⁵, —C(O)OR⁶⁵, or —C₂-C₆-alkenyl-C(═O)R⁶⁵,
C₁-C₆-alkyl, C₂-C₆-alkenyl or C₂-C₆-alkynyl, each of which may optionally be substituted with one or more substituents selected from R⁶⁷,
aryl, aryloxy, aryloxycarbonyl, aroyl, arylsulfanyl, aryl-C₁-C₆-alkoxy, aryl-C₁-C₆-alkyl, aryl-C₂-C₆-alkenyl, aroyl-C₂-C₆-alkenyl, aryl-C₂-C₆-alkynyl, heteroaryl, heteroaryl-C₁-C₆-alkyl, heteroaryl-C₂-C₆-alkenyl or heteroaryl-C₂-C₆-alkynyl,
of which the cyclic moieties optionally may be substituted with one or more substituents selected from R⁶⁸,
   R⁶⁵ and R⁶⁶ are independently selected from hydrogen, OH, CF₃, C₁-C₁₂-alkyl, aryl-C₁-C₆-alkyl, —C(═O)—R⁶⁹, aryl or heteroaryl, wherein the alkyl groups may optionally be substituted with one or more substituents selected from R⁷⁰, and the aryl and heteroaryl groups may optionally be substituted with one or more substituents independently selected from R⁷¹; R⁶⁵ and R⁶⁶ when attached to the same nitrogen atom may form a 3 to 8 membered heterocyclic ring with the said nitrogen atom, the heterocyclic ring optionally containing one or two further heteroatoms selected from nitrogen, oxygen and sulphur, and optionally containing one or two double bonds,
   R⁶⁷ is independently selected from halogen, —CN, —CF₃, —OCF₃, —OR⁶⁵, and —NR⁶⁵R⁶⁶,
   R⁶⁸ is independently selected from halogen, —C(O)OR⁶⁵, —CH₂C(O)OR⁶⁵, —CH₂OR⁶⁵, —CN, —CF₃, —OCF₃, —NO₂, —OR⁶⁵, —NR⁶⁵R⁶⁶ and C₁-C₆-alkyl,
   R⁶⁹ is independently selected from C₁-C₆-alkyl, aryl optionally substituted with one or more halogen, or heteroaryl optionally substituted with one or more C₁-C₆-alkyl,
   R⁷⁰ is independently selected from halogen, —CN, —CF₃, —OCF₃, —OC₁-C₆-alkyl, —C(O)OC₁-C₆-alkyl, —COOH and —NH₂,
   R⁷¹ is independently selected from halogen, —C(O)OC₁-C₆-alkyl, —COOH, —CN, —CF₃, —OCF₃, —NO₂, —OH, —OC₁-C₆-alkyl, —NH₂, C(═O) or C₁-C₆-alkyl,
   or any enantiomer, diastereomer, including a racemic mixture, tautomer as well as a salt thereof with a pharmaceutically acceptable acid or base.
   Embodiment 188. A pharmaceutical composition according to embodiment 187 wherein AA is aryl, heteroaryl or aryl-C₁₋₆-alkyl-, wherein the alkyl is optionally substituted with one or more R⁶³, and the aryl or heteroaryl is optionally substituted with one or more substituents independently selected from R⁶⁴.
   Embodiment 189. A pharmaceutical composition according to embodiment 188 wherein AA is aryl or heteroaryl optionally substituted with one or more substituents independently selected from R⁶⁴.
   Embodiment 190. A pharmaceutical composition according to embodiment 189 wherein AA is ArG1 or Het1 optionally substituted with one or more substituents independently selected from R⁶⁴.
   Embodiment 191. A pharmaceutical composition according to embodiment 190 wherein AA is ArG1 or Het2 optionally substituted with one or more substituents independently selected from R⁶⁴.
   Embodiment 192. A pharmaceutical composition according to embodiment 191 wherein AA is ArG1 or Het3 optionally substituted with one or more substituents independently selected from R⁶⁴.
   Embodiment 193. A pharmaceutical composition according to embodiment 192 wherein AA is phenyl, naphtyl, anthryl, carbazolyl, thienyl, pyridyl, or benzodioxyl optionally substituted with one or more substituents independently selected from R⁶⁴.
   Embodiment 194. A pharmaceutical composition according to embodiment 193 wherein AA is phenyl or naphtyl optionally substituted with one or more substituents independently selected from R⁶⁴.
   Embodiment 195. A pharmaceutical composition according to any one of the embodiments 187 to 194 wherein R⁶⁴ is independently selected from hydrogen, halogen, —CF₃, —OCF₃, —OR⁶⁵, —NR⁶⁵R⁶⁶, C₁-C₆-alkyl, —OC(O)R⁶⁵, —OC₁-C₆-alkyl-C(O)OR⁶⁵, aryl-C₂-C₆-alkenyl, aryloxy or aryl, wherein C₁-C₆-alkyl is optionally substituted with one or more substituents independently selected from R⁶⁷, and the cyclic moieties optionally are substituted with one or more substituents independently selected from R⁶⁸.
   Embodiment 196. A pharmaceutical composition according to embodiment 195 wherein R⁶⁴ is independently selected from halogen, —CF₃, —OCF₃, —OR⁶⁵, —NR⁶⁵R⁶⁶, methyl, ethyl, propyl, —OC(O)R⁶⁵, —OCH₂—C(O)OR⁶⁵, —OCH₂—CH₂—C(O)OR⁶⁵, phenoxy optionally substituted with one or more substituents independently selected from R⁶³.
   Embodiment 197. A pharmaceutical composition according to any one of the embodiments 187 to 196 wherein R⁶⁵ and R⁶⁶ are independently selected from hydrogen, CF₃, C₁-C₁₂-alkyl, aryl, or heteroaryl optionally substituted with one or more substituents independently selected from R⁷¹.
   Embodiment 198. A pharmaceutical composition according to embodiment 197 wherein R⁶⁵ and R⁶⁶ are independently hydrogen, C₁-C₁₂-alkyl, aryl, or heteroaryl optionally substituted with one or more substituents independently selected from R⁷¹.
   Embodiment 199. A pharmaceutical composition according to embodiment 198 wherein R⁶⁵ and R⁶⁶ are independently hydrogen, methyl, ethyl, propyl, butyl, 2,2-dimethyl-propyl, ArG1 or Het1 optionally substituted with one or more substituents independently selected from R⁷¹.
   Embodiment 200. A pharmaceutical composition according to embodiment 199 wherein R⁶⁵ and R⁶⁶ are independently hydrogen, methyl, ethyl, propyl, butyl, 2,2-dimethyl-propyl, ArG1 or Het2 optionally substituted with one or more substituents independently selected from R⁷¹.
   Embodiment 201. A pharmaceutical composition according to embodiment 200 wherein R⁶⁵ and R⁶⁶ are independently hydrogen, methyl, ethyl, propyl, butyl, 2,2-dimethyl-propyl, ArG1 or Het3 optionally substituted with one or more substituents independently selected from R⁷¹.
   Embodiment 202. A pharmaceutical composition according to embodiment 201 wherein R⁶⁵ and R⁶⁶ are independently hydrogen, methyl, ethyl, propyl, butyl, 2,2-dimethyl-propyl, phenyl, naphtyl, thiadiazolyl optionally substituted with one or more R⁷¹ independently; or isoxazolyl optionally substituted with one or more substituents independently selected from R⁷¹.
   Embodiment 203. A pharmaceutical composition according to any one of the embodiments 187 to 202 wherein R⁷¹ is halogen or C₁-C₈-alkyl.
   Embodiment 204. A pharmaceutical composition according to embodiment 203 wherein R⁷¹ is halogen or methyl.
   Embodiment 205. A pharmaceutical composition according to embodiment 1 wherein the ligand for the His^{B10} anion site is the SCN⁻ anion.
   Embodiment 206. A pharmaceutical composition according to embodiment 1 wherein the ligand for the His^{B10} anion site is the Cl⁻ anion.

In another embodiment the pharmaceutical composition is suitable for administration by injection or infusion. In another embodiment the pharmaceutical composition is suitable for subcutaneous administration. In another embodiment the pharmaceutical composition is suitable for intramuscular administration. In another embodiment the pharmaceutical composition is suitable for intravenous administration.

In one embodiment the present invention relates to a pharmaceutical composition according to any one of the embodiments above wherein said insulinotropic peptide is GLP-1(7-37), a GLP-1(7-37) analogue, a derivative of GLP-1(7-37), or a derivative of a GLP-1(7-37) analogue. In another embodiment hereof said GLP-1(7-37) analogue is selected from the group consisting of Arg³⁴-GLP-1(7-37), Gly⁸-GLP-1(7-36)-amide, Gly⁸-GLP-1(7-37), Val⁸-GLP-1(7-36)-amide, Val⁸-GLP-1(7-37), Val⁸Asp²²-GLP-1(7-36)-amide, Val⁸Asp²²-GLP-1(7-37), Val⁸Glu²²-GLP-1(7-36)-amide, Val⁸Glu²²-GLP-1(7-37), Val⁸Lys²²-GLP-1(7-36)-amide, Val⁸Lys²²-GLP-1(7-37), Val⁸Arg²²-GLP-1(7-36)-amide, Val⁸Arg²²-GLP-1(7-37), Val⁸His²²-GLP-1(7-36)-amide, Val⁸His²²-GLP-1(7-37), Val⁸Trp¹⁹Glu²²-GLP-1(7-37), Val⁸Glu²²Val²⁵-GLP-1(7-37), Val⁸Tyr¹⁶Glu²²-GLP-1(7-37), Val⁸Trp¹⁶Glu²²-GLP-1(7-37), Val⁸Leu¹⁶Glu²²-GLP-1(7-37), Val⁸Tyr¹⁸Glu²²-GLP-1(7-37), Val⁸Glu²²His³⁷-GLP-1(7-37), Val Glu²²Ile³³-GLP-1(7-37), Val⁸Trp¹⁶Glu²²Val²⁵Ile³³-GLP-1(7-37), Val⁸Trp¹⁶Glu²²Ile³³-GLP-1(7-37), Val⁸Glu²²Val²⁵Ile³³-GLP-1(7-37), Val⁸Trp¹⁶Glu²²Val²⁵-GLP-1(7-37), analogues thereof and derivatives of any of these.

In another embodiment according to any one of the embodiments above said insulinotropic peptide has a Glu residue in position 22. In another embodiment according to any one of the embodiments above said insulinotropic peptide has a L-histidine residue in position 8. In another embodiment according to any one of the embodiments above said insulinotropic peptide has a Val residue in position 8. In another embodiment according to any one of the embodiments above said derivative of a GLP-1(7-37) analogue is GLP-1(7-36)-amide.

In another embodiment according to any one of the embodiments above the present invention relates to a pharmaceutical composition wherein said insulinotropic peptide is a derivative of GLP-1(7-37) or a derivative of a GLP-1(7-37) analogue having a lysine residue, such as one lysine, wherein a lipophilic substituent optionally via a spacer is attached to the epsilon amino group of said lysine. In one embodiment according to any one of the embodiments above said lipophilic substituent has from 8 to 40 carbon atoms, preferably from 8 to 24, eg 12-18. In another embodiment according to any one of the embodiments above said spacer is present and is selected from an amino acid, eg. beta-Ala, L-Glu, aminobutyroyl. In another embodiment according to any one of the embodiments above said insulinotropic peptide is a dipeptidyl aminopeptidase IV protected GLP-1 compound. In another embodiment according to any one of the embodiments above said insulinotropic peptide is a plasma stable GLP-1 compound. In another embodiment according to any one of the embodiments above said derivative of a GLP-1(7-37) analogue is Arg³⁴, Lys²⁶(N^{ε}-(γ-Glu(N^{α}-hexadecanoyl)))GLP-1 (7-37). In another embodiment according to any one of the embodiments above said insulinotropic peptide has from 27 to 43 amino acid residues, preferable from 28 to 38 amino acid residues, even more preferable from 30 to 34 amino acid residues.

In another embodiment according to any one of the embodiments above the concentration of said insulinotropic peptide in said pharmaceutical composition is from about 1 mg/mL to about 25 mg/mL, from about 2 mg/mL to about 15 mg/mL, from about 5 mg/mL to about 12 mg/mL, or from about 8 mg/mL to about 11 mg/mL. In another embodiment according to any one of the embodiments above the concentration of said insulinotropic peptide in said pharmaceutical composition is from about 5 mg/mL to about 7.5 mg/mL.

In another embodiment according to any one of the embodiments above the present invention relates to a pharmaceutical composition wherein said insulinotropic peptide is exendin-4, an exendin-4 analogue, a derivative of exendin-4, or a derivative of an exendin-4 analogue. In one embodiment according to any one of the embodiments above said insulinotropic peptide is exendin-4. In another embodiment according to any one of the embodiments above said exendin-4 analogue is exendin-3 or ZP-10 (HGEGTFTSDLSKQMEEEAVRL-FIEWLKNGGPSSGAPPSKKKKKK-NH2).

In another embodiment according to any one of the embodiments above said derivative of an exendin-4 analogue is an acylated exendin-4 analogue or a pegylated exendin-4 analogue. In another embodiment according to any one of the embodiments above said insulinotropic peptide is a derivative of exendin-4 or a derivative of an exendin-4 analogue having a lysine residue, such as one lysine, wherein a lipophilic substituent optionally via a spacer is attached to the epsilon amino group of said lysine. In another embodiment according to any one of the embodiments above said lipophilic substituent has from 8 to 40 carbon atoms, preferably from 8 to 24, eg 12-18. In another embodiment according to any one of the embodiments above said spacer is present and is selected from an amino acid, eg. beta-Ala, L-Glu, aminobutyroyl. In another embodiment according to any one of the embodiments above said insulinotropic peptide is a dipeptidyl aminopeptidase IV protected exendin-4 compound. In another embodiment according to any one of the embodiments above said insulinotropic peptide is a plasma stable exendin-4 compound. In another embodiment according to any one of the embodiments above said insulinotropic peptide has from 30 to 48 amino acid residues, from 33 to 45 amino acid residues, preferable from 35 to 43 amino acid residues, even more preferable from 37 to 41 amino acid residues. In another embodiment according to any one of the embodiments above the concentration of said insulinotropic peptide in said pharmaceutical composition is from about 5 μg/mL to about 10 mg/mL, from about 5 μg/mL to about 5 mg/mL, from about 0.1 mg/mL to about 3 mg/mL, or from about 0.2 mg/mL to about 1 mg/mL.

In one embodiment the present invention relates to a pharmaceutical composition according to any one of the embodiments above wherein the insulin peptide is human insulin, which may be naturally produced insulin or recombinantly produced insulin. Recombinant human insulin may be produced in any suitable host cell, for example the host cells may be bacterial, fungal (including yeast), insect, animal or plant cells. Many insulin compounds have been disclosed in the literature, and may be employed in a pharmaceutical composition according to any the present invention.

The following patent documents are disclosures of insulin compounds that may be employed in a pharmaceutical composition according to any of the above embodiments of the present invention.

With “desB30” or “B(1-29)” is meant a natural insulin B chain or an analogue thereof lacking the B30 amino acid residue, “B(1-26)” is a peptide chain consisting of the first 26 amino acid residues of the B chain of human insulin counted from the N-terminal end of the B chain or an analogue thereof, “A(1-21)” means the natural insulin A chain or an analogue thereof and A(1-20) means the first 20 natural amino acid residues of the A chain of human insulin or an analogue thereof. The amino acid residues are indicated in the three letter amino acid code or the one letter amino code.

With “B1”, “A1” etc. is meant the amino acid residue in position 1 in the B chain of insulin (counted from the N-terminal end) and the amino acid residue in position 1 in the A chain of insulin (counted from the N-terminal end), respectively.

WO 97/31022 (Novo Nordisk), which is incorporated herein by reference, discloses insulin compounds with a protracted activity profile wherein the amino group of the N-terminal amino acid of the B-chain and/or the ε-amino group of Lys^{B29} has a carboxylic acid containing lipophilic substituent. Particular mention is made of N^{εB29}—(CO—(CH₂)₁₄—COOH) human insulin; N^{εB29}—(Co-(CH₂)₁₆—COOH) human insulin; N^{εB29}—(CO—(CH₂)₁₈—COOH) human insulin; N^{εB29}—(CO—(CH₂)₂₀—COOH); N^{εB29}—(CO—(CH₂)₂₂—COOH) human insulin; N^{εB29}—(CO—(CH₂)₁₄—COOH) Asp^{B28}-human insulin; N^{εB29}—(CO—(CH₂)₁₆—COOH) Asp^{B28}-human insulin; N^{εB29}—(CO—(CH₂)₁₈—COOH) Asp^{B28}-human insulin; N^{εB29}—(CO—(CH₂)₂₀—COOH) Asp^{B28}-human insulin; N^{εB29}—(CO—(CH₂)₂₂—COOH) Asp^{B28}-human insulin; N^{εB30}-(CO—(CH₂)₁₄—COOH) Thr^{B29}Lys^{B30}-human insulin; N^{εB30}-(CO—(CH₂)₁₆—COOH) Thr^{B29}Lys^{B30}-human insulin; N^{εB30}—(CO(CH₂)₁₈—COOH) Thr^{B29}Lys^{B30}-human insulin; N^{εB30}-(CO—(CH₂)₂₀—COOH) Thr^{B29}Lys^{B30}-human insulin; N^{εB30}—(CO—(CH₂)₂₂—COOH) Thr^{B29}Lys^{B30}-human insulin; N^{εB28}-(CO—(CH₂)₁₄—COOH) Lys^{B28}Pro^{B29}-human insulin; N^{εB28}-(CO—(CH₂)₁₆—COOH) Lys^{B21}Pro^{B29}-human insulin; N^{εB28}-(CO—(CH₂)₁₈—COOH) Lys^{B28}Pro^{B29}-human insulin; N^{εB28}—(CO—(CH₂)₂₀—COOH) Lys^{B30}-human insulin; N^{εB28}-(CO—(CH₂)₂₂—COOH) Lys^{B28}Pro^{B29}-human insulin; N^{εB29}—(CO—(CH₂)₁₄—COOH) desB30 human insulin; N^{εB29}—(CO—(CH₂)₁₆—COOH) desB30 human insulin; N^{εB29}—(CO—(CH₂)₁₈—COOH) desB30 human insulin; N^{εB29}—(CO—(CH₂)₂₀—COOH) desB30 human insulin; and N^{εB29}-(CO—(CH₂)₂₂COOH) desB30 human insulin.

WO 96/29344 (Novo Nordisk), which is incorporated herein by reference, discloses insulin compounds with a protracted activity profile wherein either the amino group of the N-terminal amino acid of the B-chain has a lipophilic substituent comprising from 12 to 40 carbon atoms attached, or wherein the carboxylic acid group of the C-terminal amino acid of the B-chain has a lipophilic substituent comprising from 12 to 40 carbon atoms attached.

WO 95/07931 (Novo Nordisk), which is incorporated herein by reference, discloses insulin compounds with a protracted activity profile, wherein the F-amino group of Lys^{B29} has a lipophilic substituent. Particular mention is made of N^{εB29}-tridecanoyl des(B30) human insulin, N^{εB29}-tetradecanoyl des(B30) human insulin, N^{εB29}-decanoyl des(B30) human insulin, N^{εB29}-dodecanoyl des(B30) human insulin, N^{εB29}-tridecanoyl Gly^{A21} des(B30) human insulin, N^{εB29}-tetradecanoyl Gly^{A21} des(B30) human insulin, N^{εB29}-decanoyl Gly^{A21} des(B30) human insulin, N^{εB29}-dodecanoyl Gly^{A21} des(B30) human insulin, N^{εB29}-tridecanoyl Gly^{A21} Gln^{B3} des(B30) human insulin, N^{εB29}-tetradecanoyl Gly^{A21} Gln^{B3} des(B30) human insulin, N^{εB29}-decanoyl Gly^{A21} Gln^{B3} des(B30) human insulin, N^{εB29}-dodecanoyl Gly^{A21} Gln^{B3} des(B30) human insulin, N^{εB29}-tridecanoyl Ala^{A21} des(B30) human insulin, N^{εB29}-tetradecanoyl Ala^{A21} des(B30) human insulin, N^{εB29}-decanoyl Ala^{A21} des(B30) human insulin, N^{εB29}-dodecanoyl Ala^{A21} des(B30) human insulin, N^{εB29}-tridecanoyl Ala^{A21} Gln^{E3} des(B30) human insulin, N^{εB29}-tetradecanoyl Ala^{A21} Gln^{B3} des(B30) human insulin, N^{εB29}-decanoyl Ala^{A21} Gln^{B3} des(B30) human insulin, N^{εB29}-dodecanoyl Ala^{A21} Gln^{B3} des(B30) human insulin, N^{εB29}-tridecanoyl Gln^{B3} des(B30) human insulin, N^{εB29}-tetradecanoyl Gln^{B3} des(B30) human insulin, N^{εB29}-decanoyl Gln^{B3} des(B30) human insulin, N^{εB29}-dodecanoyl Gln^{B3} des(B30) human insulin, N^{εB29}-tridecanoyl Gly^{A21} human insulin, N^{εB29}-tetradecanoyl Gly^{A21} human insulin, N^{εB29}-decanoyl Gly^{A1} human insulin, N^{εB29}-dodecanoyl Gly^{A21} human insulin, N^{εB29}-tridecanoyl Gly^{A21} Gln^{B3} human insulin, N^{εB29}-tetradecanoyl Gly^{A21} Gln^{B3} human insulin, N^{εB29}-decanoyl Gly^{A21} Gln^{B3} human insulin, N^{εB29}-dodecanoyl Gly^{A21} Gln^{B3} human insulin, N^{εB29}-tridecanoyl Ala^{A21} human insulin, N^{εB29}-tetradecanoyl Ala^{A21} human insulin, N^{εB29}-decanoyl Ala^{A21} human insulin, N^{εB29}-dodecanoyl Ala^{A21} human insulin, N^{εB29}-tridecanoyl Ala^{A21} Gln^{B3} human insulin, N^{εB29}-tetradecanoyl Ala^{A21} Gln³ human insulin, N^{εB29}-decanoyl Ala^{A21}Gln^{B3} human insulin, N^{εB29}-dodecanoyl Ala^{A21} Gln^{E3} human insulin, N^{εB29}-tridecanoyl Gln^{B3} human insulin, N^{εB29}-tetradecanoyl Gln^{B3} human insulin, N^{εB29}-decanoyl Gln^{B3} human insulin, N^{εB29}-dodecanoyl Gln^{B3} human insulin, N^{εB29}-tridecanoyl Glu^{B30} human insulin, N^{εB29}-tetradecanoyl Glu^{B30} human insulin, N^{εB29}-decanoyl Glu^{B30} human insulin, N^{εB29}-dodecanoyl Glu^{B30} human insulin, N^{εB29}-tridecanoyl Gly^{A21} Glu^{B30} human insulin, N^{εB29}-tetradecanoyl Gly^{A21} Glu^{B30} human insulin, N^{εB29}-decanoyl Gly¹ Glu^{B30} human insulin, N^{εB29}-dodecanoyl Gly¹¹ Glu^{B30} human insulin, N^{εB29}-tridecanoyl Gly^{A21} Gln^{B3} Glu^{B30} human insulin, N^{εB29}-tetradecanoyl Gly¹ Gln^{B3} Glu^{B30} human insulin, N^{εB29}-decanoyl Gly^{A21} Gln^{B3} Glu^{B30} human insulin, N^{εB29}-dodecanoyl Gly^{A21} Gln^{B3} Glu^{B30} human insulin, N^{εB29}-tridecanoyl Ala^{A21} Glu^{B30} human insulin, N^{εB29}-tetradecanoyl Ala^{A2} Glu^{B30} human insulin, N^{εB29}-decanoyl Ala^{A21} Glu^{B30} human insulin, N^{εB29}-dodecanoyl Ala^{A21} Glu^{B30} human insulin, N^{εB29}-tridecanoyl Ala^{A21} Gln^{B3} Glu^{B30} human insulin, N^{εB29}-tetradecanoyl Ala^{A21} Gln^{B3} Glu^{B30} human insulin, N^{εB29}-decanoyl Ala^{A2} Gln^{E3} Glu^{B30} human insulin, N^{εB29}-dodecanoyl Ala^{A21} Gln^{B3} Glu^{B30} human insulin, N^{εB29}-tridecanoyl Gln^{B3} Glu^{B30} human insulin, N^{εB29}-tetradecanoyl Gln^{B3} Glu^{B30} human insulin, N^{εB29}-decanoyl Gln^{B3} Glu^{B30} human insulin and N^{εB29}-dodecanoyl Gln^{B3} Glu^{B30} human insulin.

WO 97/02043 (Novo Nordisk), which is incorporated herein by reference discloses hormonally inactive insulin compounds which are useful in insulin prophylaxis, and in particular such analogues of human insulin are selected from amongst desA1 human insulin; des(A1-A2) human insulin; des(A1-A3) human insulin; desA21 human insulin; des(B1-B5) human insulin; des(B1-B6) human insulin; des(B23-B30) human insulin; des(B24-B30) human insulin; des(B25-B30) human insulin; Gly^{A2} human insulin; Ala^{A2} human insulin; Nle^{A2} human insulin; Thr human insulin; Pros human insulin; D-allo Ile^{A2} human insulin; Nva^{A3} human insulin; Nle^{A3} human insulin; Leu^{A3} human insulin; Val^{A2},Ile^{A3} human insulin; Abu^{A2},Abu^{A3} human insulin; Gly^{A2},Gly^{A3} human insulin; D-Cys^{A6} human insulin; D-Cys^{A6},D-Cys^{A11} human insulin; Ser^{A6},Ser^{A11},des(A8-A10) human insulin; D-Cys^{A7} human insulin; D-Cys^{A11} human insulin; Leu^{A19} human insulin; Gly^{B6} human insulin; Glu^{B12} human insulin; Asn^{B12} human insulin; Phe^{B12} human insulin; D-Ala^{B12} human insulin; and Asp^{B25} human insulin are applicable in the methods of the present invention.

WO 92/15611 (Novo Nordisk), which is incorporated herein by reference, discloses analogues of human insulin with a fast association rate constants in the insulin receptor binding process and characterised by comprising a tyrosine in position A13 and/or a phenylalanine, tryptophane or tyrosine in position B17. In particular, such analogues are selected from amongst Tyr^{A13} human insulin, Phe^{B17} human insulin, Trp^{B17} human insulin, Tyr^{B17} human insulin, Tyr^{A13},Phe^{B17} human insulin, Tyr^{A13},Trp^{B17} human insulin, Tyr^{A13},Tyr^{B17} human insulin, Phe^{A13},Phe^{B17} human insulin, Phe^{A13},Trp^{B17} human insulin, Phe^{A13},Tyr^{B17} human insulin, Trp^{A13},Phe^{B17} human insulin, Trp^{A13},Trp^{B17} human insulin and Trp^{A13},Tyr^{B17} human insulin.

WO 92/00322 (Novo Nordisk), which is incorporated herein by reference, discloses analogues of human insulin which are capable of being targeted to specific tissues, and which are characterized by having in the A13 position and/or in the B17 position in the insulin molecule a naturally occurring amino acid residue different from leucine and/or by having in the B18 position in the insulin molecule a naturally occurring amino acid residue different from valine. In particular, such analogues are selected from amongst Ala^{B17} human insulin, Ala^{B18} human insulin, Asn^{A13} human insulin, Asn^{A13},Ala^{B17} human insulin, Asn^{A13},Asp^{B17} human insulin, Asn^{A13},Glu B17 human insulin, Asn^{B18} human insulin, Asp^{A13} human insulin, Asp^{A13},Ala^{B17} human insulin, Asp^{A13},Asp^{B17} human insulin, Asp^{A13},Glu^{B17} human insulin, Asp^{B18} human insulin, Gln^{A13} human insulin, Gln^{A13},Ala^{B17} human insulin, Gln^{A13},Asp^{B17} human insulin, Gln^{B18} human insulin, Glu^{A13} human insulin, Glu^{A13},Ala^{B17} human insulin, Glu^{A13},Asp^{B17} human insulin, Glu^{A13},Glu^{B17} human insulin, Glu^{B18} human insulin, Gly^{A13} human insulin, Gly^{A13},Ala^{B17} human insulin, Gly^{A13},Asn^{B17} human insulin, Gly^{A13},Asp^{B17} human insulin, Gly^{A13},Glu^{B17} human insulin, Gly^{B18} human insulin, Ser^{A13} human insulin, Ser^{A13},Gln^{A17},Glu^{B10},Gln^{B17}-des(Thr^{B30}) human insulin, Ser^{A13},Ala^{B17} human insulin, Ser^{A13},Asn^{B17} human insulin, Ser^{A13},Asp^{B17} human insulin, Ser^{A13},Gln^{B17} human insulin, Ser^{A13},Glu^{B17} human insulin, Ser^{A13},Thr^{B17} human insulin, Ser^{B14},Asp^{B17} human insulin, Ser^{B18} human insulin, Thr^{A13} human insulin or Thr^{B18} human insulin.

WO 90/01038 (Novo Nordisk), which is incorporated herein by reference, discloses analogues of human insulin with high biological activity and characterized by having Phe^{B25} substituted by His or Tyr, by having substitutions in one or more of positions A4, A8, A17, A21, B9, B10, B12, B13, B21, B26, B27, B28 and B30, and by having the amino acid residue at position B30 optionally absent. In particular, such analogues are selected from amongst Tyr^{B25} human insulin, Tyr^{B25},Asp^{B28} human insulin, His^{B25} human insulin, His^{B25},Asp^{B28} human insulin, Tyr^{B25} human insulin-B30-amide and His^{B25} human insulin-B30-amide.

WO 86/05496 (Nordisk Gentofte) discloses analogues of human insulin with a protracted action and characterized by having a blocked B30 carboxylic group, and by having one to four blocked carboxylic groups in the amino acid residues at positions A4, A17, A21, B13 and B21. In particular, such analogues are selected from amongst insulin-B30-octyl ester, insulin-B30-dodecyl amide, insulin-B30-hexadecyl amide, insulin-(B21,B30)-dimethyl ester, insulin(B17,B30)-dimethyl ester, insulin-(A4,B30) diamide, insulin-A17amide-B30-octyl ester, insulin(A4,B13)-diamide-B30-hexylamide, insulin-(A4,A17,B21,B30)-tetraamide, insulin-(A17,B30)diamide, A4-Ala-insulin-B30-amide and B30-Leu-insulin-(A4,B30)-diamide.

WO 86/05497(Nordisk Gentofte), which is incorporated herein by reference, discloses insulin compounds in which one or more of the four amino acid residues in positions A4, A17, B13 and B21 comprises an uncharged side chain. Particular mentioning is made of human insulin A17-Gln, human insulin A4-Gln, porcine insulin B21-Gln, human insulin B13-Gln, human insulin (A17,B21)-Gln, human insulin A4-Ala, human insulin B21-Thr, human insulin B13-Val, human insulin-Thr-A17-Gln, human insulin B21-methyl ester and human insulin A17-methyl ester.

WO 92/00321 (Novo Nordisk), which is incorporated herein by reference, discloses insulin compounds with prolonged activity wherein a positive charge in the N-terminal end of the B-chain has been introduced. Particular mentioning is made of Arg^{B5},Ser^{A21},Thr^{B30}-NH₂ human insulin, Arg^{B5},Pro^{B6},Ser^{A21},Thr^{B30}-NH₂ human insulin, Arg^{B5},Gly^{A21},Thr^{B30}-NH₂ human insulin, Arg^{B5},Pro^{B6},Gly^{A21},Thr^{B30}-NH₂ human insulin, Arg^{B2},Ser^{A21}Thr^{B30}-NH₂ human insulin, Arg^{B2},Pro^{B3},Ser^{A21},Thr^{B30}-NH₂ human insulin, Arg^{B2},Gly^{A21},Thr^{B30}-NH₂ human insulin, Arg^{B2},Pro^{B3},Gly^{A21},Thr^{B30}-NH₂ human insulin, Arg^{B2},Arg^{B3},Ser^{A21},Thr^{B30}-NH₂ human insulin, Arg^{B2},Arg^{B3},Ser^{A21} human insulin, Arg^{B4},Pro^{B5},Ser^{A21},Thr^{B30}-NH₂ human insulin, Arg^{B4},Arg^{B5},Pro^{B6},Gly^{A21},Thr^{B30} human insulin, Arg^{B3},Gly^{A21},Thr^{B30}-NH₂ human insulin, Arg^{B3},Ser²¹,Thr^{B30}-NH₂ human insulin, Arg^{B4},Gly^{A2},Thr^{B30}-NH₂ human insulin, Arg^{B4},Ser^{A21},Thr^{B30}-NH₂ human insulin and Arg^{B1},Pro^{B2},Gly^{A21},Thr^{B30}-NH₂ human insulin.

WO 90/07522 (Novo Nordisk), which is incorporated herein by reference, discloses insulin compounds exhibiting a low ability to associate in solution wherein there is a positively charged amino acid residue, i.e. Lys or Arg in the position B28. Particular mention is made of des[Phe^{B25}]-human insulin, des[Tyr^{B26}]-human insulin, des[Thr^{B27}]-human insulin, des[Pro^{B28}]-human insulin, des[Phe^{B25}]-porcine insulin, des[Pro^{B28}]-porcine insulin, des[Pro^{B28}]-rabbit insulin, des[Phe^{B25}],des[Thr^{B30}]-human insulin, des[Tyr^{B26}],des[Thr^{B30}]-human insulin, [Ser^{A21}]-des-[Pro^{B28}]-human insulin, [Gly^{A21}]-des[Pro^{B28}]-human insulin, [Gly^{A21}]-des[Phe^{B25}]-human insulin, [Asp^{A21}]-des[Phe^{B25}]-human insulin, [His^{B21}]-des[Tyr^{B26}], des[Thr^{B30}]-human insulin, [Asn^{B25}]-des[Tyr^{B26}],des[Thr^{B30}]-human insulin, [Asp^{A1}]-des[Phe^{B25}],des[Thr^{B30}]-human insulin, [Asp^{B28}]-des[Phe^{B25}]-human insulin, [Asp^{B3}]-des[Phe^{B25}]-human insulin, [Lys^{B28}]-human insulin, [Lys^{B28},Thr^{B29}]-human insulin and [Arg^{B28}]-des[Lys^{B29}]-human insulin.

WO 90/11290 (Novo Nordisk), which is incorporated herein by reference discloses insulin compounds with a prolonged activity. Particular mention is made of [Arg^{A0}]-human insulin(B30-amide), [Arg^{A0},Gln^{B13}]-human insulin-(B30-amide), [Arg^{A0},Gln^{A4},Asp^{A21}]-human insulin(B30-amide), [Arg^{A0},Ser^{A21}]-human insulin-(B30-amide) and [Arg^{A0},Arg^{B27}]-des[Thr^{B30}]-human insulin.

WO 90/10645 (Novo Nordisk), which is incorporated herein by reference discloses glycosylated insulins. Particular mention is made of Phe(B1) glucose human insulin, Phe(B1) mannose human insulin, Gly(A1) mannose human insulin, Lys(B29) mannose human insulin, Phe(B1) galactose human insulin, Gly(A1) galactose human insulin, Lys(B29) galactose human insulin, Phe(B1) maltose human insulin, Phe(B1) lactose human insulin, Gly(A1) glucose human insulin, Gly(A1) maltose human insulin, Gly(A1) lactose human insulin, Lys(B29) glucose human insulin, Lys(B29) maltose human insulin, Lys(B29) lactose human insulin, Gly(A1),Phe(B1) diglucose human insulin, Gly(A1),Lys(B29) diglucose human insulin, Phe(B1),Lys(B29) diglucose human insulin, Phe(B1) isomaltose human insulin, Gly(A1) isomaltose human insulin, Lys(B29) isomaltose human insulin, Phe(B1) maltotriose human insulin, Gly(A1) maltotriose human insulin, Lys(B29) maltotriose human insulin, Gly(A1),Phe(B1) dimaltose human insulin, Gly(A1),Lys(B29) dimaltose human insulin, Phe(B1),Lys(B29) dimaltose human insulin, Gly(A1),Phe(B1) dilactose human insulin, Gly(A1),Lys(B29) dilactose human insulin, Phe(B1),Lys(B29) dilactose human insulin, Gly(A1),Phe(B1) dimaltotriose human insulin, Gly(A1),Lys(B29) dimaltotriose human insulin, Phe(B1),Lys(B29) dimaltotriose human insulin, Phe(B1),Gly(A1) dimannose human insulin, Phe(B1),Lys(B29) dimannose human insulin, Gly(A1),Lys(B29) dimannose human insulin, Phe(B1),Gly(A1) digalactose human insulin, Phe(B1),Lys(B29) digalactose human insulin, Gly(A1),Lys(B29) digalactose human insulin, Phe(B1),Gly(A1) diisomaltose human insulin, Phe(B1),Lys(B29) diisomaltose human insulin, Gly(A1),Lys(B29) diisomaltose human insulin, Phe(B1) glucose [Asp^{B10}] human insulin and Gly(A1),Phe(B1) diglucose [Asp^{B10}] human insulin.

WO 88/065999 (Novo Nordisk), which is incorporated herein by reference, discloses stabilized insulin compounds, wherein Ans^{21A} has been substituted with other amino acid residues. Particular mentioning is made of Gly^{A21} human insulin, Ala^{A21} human insulin, Ser^{A21} human insulin, Thr^{A21} human insulin and hSer^{A21} human insulin.

EP 254516 (Novo Nordisk), which is incorporated herein by reference, discloses insulin compounds with a prolonged action, wherein basic amino acid residues have been substituted by neutral amino acid residues. Particular mention is made of Gly^{A21},Lys^{B27},Thr^{B30}-NH₂ human insulin, Ser^{A21},Lys^{B27},Thr^{B30}-NH₂ human insulin, Thr^{A21},Lys^{B27},Thr^{B30}-NH₂ human insulin, Ala^{B21},Lys^{B27},Thr^{B30}-NH₂ human insulin, His^{A21},Lys^{B27},Thr^{B30}-NH₂ human insulin, Asp^{B21},Thr^{B30}-NN₂ human insulin, Gly^{A21},Arg^{B21},Thr^{B30}-NH₂ human insulin, Ser^{A21},Arg^{B27}, Thr^{B30}-NH₂ human insulin, Thr^{A21},Arg^{B27},Thr^{B30}-NH₂ human insulin, Ala^{B21},Arg^{B27},Thr^{B30}-NH₂ human insulin, His^{A21},Arg^{B27},Thr^{B30}-NH₂ human insulin, Asp^{B21},Arg^{B27},Thr^{B30}-NH₂ human insulin, Gln^{B13},Gly^{A21},Arg^{B27},Thr^{B30}-NH₂ human insulin, Gln^{B13},Ser^{A21},Thr^{B30}-NH₂ human insulin, Gln^{B13},Ser^{A21},Arg^{B27},Thr^{B30}-NH₂ human insulin, Gln^{B13},Thr^{A21},Arg^{B27},Thr^{B30}-NH₂ human insulin, Gln^{B13},Ala^{A21},Arg^{B27},Thr^{B30}-NH₂ human insulin, Gln^{B13},His^{A21},Arg^{B27},Thr^{B30}-NH₂ human insulin, Gln^{B13},Asp^{A21},Arg^{B27},Thr^{B30}-NH₂ human insulin, Gln^{B13},Gly^{A21},Thr^{B30}NH₂ human insulin, Gln^{B13},Ser^{A21},Lys^{B27},Thr^{B30}-NH₂ human insulin, Gln^{B13},Thr^{A21},Lys^{B27},Thr^{B30}-NH₂ human insulin, Gln^{B13},Ala^{A21},Lys^{B27},Thr^{B30}-NH₂ human insulin, Gln^{B13},His^{A21},Lys^{B27},Thr^{B30}-NH₂ human insulin, Gln^{B13},Asp^{A21},Lys^{B27},Thr^{B30}-NH₂ human insulin, Asn^{A21},Lys^{B27} human insulin, Ser^{A21},Lys^{B27} human insulin, Thr^{A21},Lys^{B27} human insulin, Ala^{A21},Lys^{B27} human insulin, His^{A21},Lys^{B27} human insulin, Asp^{A21}_{,Lys}^{B27} human insulin, Gly^{A21}_{,Lys}^{B27} human insulin, Asn^{A21},Arg^{B27} human insulin, Ser^{A21},Arg^{B27} human insulin, Thr^{A21},Arg^{B27} human insulin, Ala^{A21},Arg^{B27} human insulin, His^{A21},Arg^{B27} human insulin, Asp^{A21},Arg^{B27} human insulin, Gly^{A21},Arg^{B27} human insulin, Gln^{A17},Asn^{A21},Arg^{B27} human insulin, Gln^{A17},Ser^{A21},Arg^{B27} human insulin, Gln^{A17},Thr^{A21},Arg^{B27} human insulin, Gln^{A17},Ala^{A21},Arg^{B27} human insulin, Gln^{A17},His^{A21},Arg^{B27} human insulin, Gln^{A17},Asp^{A21},Arg^{B27} human insulin, Gln^{A17},Gly^{A21},Arg^{B27} human insulin, Gln^{A17},Asn^{A21},Gln^{B13} human insulin, Gln^{A17},Ser^{A21},Gln^{B13} human insulin, Gln^{A17},Thr^{A21},Gln^{B13} human insulin, Gln^{A17},Ala^{A21},Gln^{B13} human insulin, Gln^{A17},His^{A21},Gln^{B13} human insulin, Gln^{A17},Asp^{A21},Gln^{B13} human insulin, Gln^{A17},His^{A21},Gln^{B13} human insulin, Arg^{A27},Asn^{A21},Gln^{B13} human insulin, Arg^{A27},Ser^{A21},Gln^{B13} human insulin, Arg^{A27},Thr^{A21},Gln^{B13} human insulin, Arg^{A27},Ala^{A21},Gln^{B13} human insulin, Arg^{A27},His^{A21},Gln^{B13} human insulin, Arg^{A27},Asp^{A21},Gln^{B13} human insulin, Arg^{A27},Gly^{A21},Gln^{B13} human insulin, Gln^{A17},Asn^{A21},Lys^{B27} human insulin, Gln^{A17},Ser^{A21},Lys^{B27} human insulin, Gln^{A17},Thr^{A21},Lys^{B27} human insulin, Gln^{A17},Ala^{A21},Lys^{B27} human insulin, Gln^{A17},His^{A21}, Lys^{B27} human insulin, Gln^{A17},Asp^{A21},Lys^{B27} human insulin, Gln^{A17},Gly^{A21},Lys^{B27} human insulin, Gln^{B13},Asn^{A21},Lys^{B27} human insulin, Gln^{B13},Ser^{A21},Lys^{B27} human insulin, Gln^{B13},Thr^{A21},Lys^{B27} human insulin, Gln^{B13},Ala^{A21},Lys^{B27} human insulin, Gln^{B13},His^{A21},Lys^{B27} human insulin, Gln^{B13},Asp^{A21},Lys^{B27} human insulin, and Gln^{B13},Gly^{A21},Lys^{B27} human insulin.

EP 214826 (Novo Nordisk), which is incorporated herein by reference, discloses rapid onset insulin compounds.

EP 194864 (Novo Nordisk), which is incorporated herein by reference, discloses insulin compounds with a prolonged action, wherein basic amino acid residues have been substituted by neutral amino acid residues. Particular mention is made of Gln^{A17},Arg^{B27},Thr^{B30}-NH₂ human insulin, Gln^{A17},Gln^{B13},Thr^{B30}-NH₂ human insulin, Gln^{A17},Lys^{B27},Thr^{B30}-NH₂ human insulin, Gln^{A17},Lys^{B27}-NH₂ human insulin, Gln^{A17}, Gln^{A17},Thr^{B30}-NH₂ human insulin, Gln^{B13},Arg^{B27},Thr^{B30}-NH₂ human insulin, Gln^{B13},Lys^{B27}Thr^{B30}-NH₂ human insulin, Gln^{B13},Lys^{B30}-NH₂ human insulin, Gln^{B13},Thr^{B30}-NH₂ human insulin, Arg^{B27},Arg^{B30}-NH₂ human insulin, Arg^{B27},Lys^{B30}-NH₂ human insulin, Arg^{B27},Thr^{B30}-NH₂ human insulin, Lys^{B27},Arg^{B30}-NH₂ human insulin, Lys^{B27},Lys^{B30}-NH₂ human insulin, Lys^{B27},Thr^{B30}-NH₂ human insulin, Lys^{B29}-NH₂,des-(B30)human insulin, Thr^{B30}-NH₂ human insulin, Lys^{B30}-NH₂ human insulin, Lys^{B30}(Lau)-NH₂ human insulin, Lys^{B30},Arg^{B31}-NH₂ human insulin, Lys^{B30},Lys^{B31}-NH₂ human insulin, Arg^{B30}-NH₂ human insulin, Arg^{B30},Arg^{B31}-NH₂ human insulin, and Arg^{B30},Lys^{B31}-NH₂ human insulin.

U.S. Pat. No. 3,528,960 (Eli Lilly), which is incorporated herein by reference, discloses N-carboxyaroyl insulin compounds in which one, two or three primary amino groups of the insulin molecule has a carboxyaroyl group.

GB Patent No. 1.492.997 (Nat. Res. Dev. Corp.), which is incorporated herein by reference, discloses insulin compounds with a carbamyl substitution at N^{εB29} with an improved profile of hypoglycaemic effect.

JP laid-open patent application No. 1-254699 (Kodama Co., Ltd.), which is incorporated herein by reference, discloses insulin compounds, wherein an alkanoyl group is bound to the amino group of Phe^{B1} or to the 6-amino group of Lys^{B29} or to both of these.

JP laid-open patent application No. 57-067548 (Shionogi), which is incorporated herein by reference, discloses insulin compounds, in which the B30 position have an amino acid having at least five carbon atoms which cannot necessarily be coded for by a triplet of nucleotides.

WO 03/053339 (Eli Lilly), which is incorporated herein by reference, discloses insulin compounds, wherein the A-chain in the N-terminal has been extended with two amino acid residues, A-1 and A0, wherein the B-chain has been extended at the N-terminal with two amino acid residues, B-1 and B0, wherein the amino acid residues at positions B28, B29 and B39 may be substituted, and wherein the ε-amino group of Lys at position B28 or B29 is covalently bound to the α-carboxyl group of a positively charged amino acid to form a Lys-Nε-aminoacid derivative. Particular mentioning is made of said analogues, wherein A-1 and B-1 are both absent, and wherein A0 represent Arg and B0 represents Arg or is absent.

Insulin compounds selected from the group consisting of:
i. An analogue of human insulin wherein position B28 is Asp, Lys, Leu, Val, or Ala and position B29 is Lys or Pro; and
ii. des(B28-B30), des(B27) or des(B30) human insulin.
   may also be employed in a pharmaceutical composition according to any of the above embodiments of the present invention, and in particular, the insulin compound wherein position B28 is Asp or Lys, and position B29 is Lys or Pro.
   des(B30) human insulin is also applicable in the methods of the present invention.

Other applicable insulin compounds are selected from the group consisting of B29-N^{ε}-myristoyl-des(B30) human insulin, B29-N^{ε}-palmitoyl-des(B30) human insulin, B29-N-myristoyl human insulin, B29-N^{ε}-palmitoyl human insulin, B28-N^{ε}-myristoyl Lys^{B28} Pro^{B29} human insulin, B28-N^{ε}-palmitoyl Lys^{B28} Pro^{B29} human insulin, B30-N^{ε}-myristoyl-Thr 29Lys^{B30} human insulin, B30-N^{ε}-palmitoyl-Thr^{B29}Lys^{B30} human insulin, B29-N^{ε}-(N-palmitoyl-γ-glutamyl)des(B30) human insulin, B29-N^{ε}-(N-lithocholyl-γ-glutamyl)-des(B30) human insulin, B29-N^{α}-(ω-carboxyheptadecanoyl)-des(B30) human insulin, B29-N^{ε}-(ω-carboxyheptadecanoyl) human insulin and B29-N^{ε}-myristoyl-des(B30) human insulin.

Other applicable insulin compounds are selected from single chain insulin compounds. By a single-chain insulin is meant a polypeptide sequence of the general structure B-C-A wherein B is the human B insulin chain or an analogue thereof, A is the human insulin A chain or an analogue and C is a peptide chain of 5-14 amino acid residues connecting B30 or with A1. If the B chain is a desB30 chain the connecting peptide will contain 6-14 amino acid residues. The single-chain insulin may be derivatized by being acylated at B29Lys or B28Lys or a Lys localized in the connecting peptide, preferably by a fatty acid group with from 6-18 carbon atoms. The single-chain insulin will contain correctly positioned disulphide bridges (three) as in human insulin that is between CysA7 and CysB7 and between CysA20 and CysB19 and an internal disulfide bridge between CysA6 and CysA11.

In one embodiment the single-chain insulin is as disclosed in EP 1,193,272, which is specifically incorporated by reference. These single-chain insulins have a modified C-peptide of 5-18 amino acids and are reported to have up to 42% insulin activity. EP 1,193,272 discloses the following modified C-peptides connecting B30 with A21: Gly-Gly-Gly-Pro-Gly-Lys-Arg, Arg-Arg-Gly-Pro-Gly-Gly-Gly, Gly-Gly-Gly-Gly-Gly-Lys-Arg, Arg-Arg-Gly-Gly-Gly-Gly-Gly, Gly-Gly-Ala-Pro-Gly-Asp-Val-Lys-Arg, Arg-Arg-Ala-Pro-Gly-Asp-Val-Gly-Gly, Gly-Gly-Tyr-Pro-Gly-Asp-Val-Lys-Arg, Arg-Arg-Tyr-Pro-Gly-Asp-Val-Gly-Gly, Gly-Gly-His-Pro-Gly-Asp-Val-Lys-Arg, and Arg-Arg-His-Pro-Gly-Asp-Val-Gly-Gly. EP 741,188, which is specifically incorporated by reference, discloses single-chain insulins with a modified C-peptide having from 10-14 amino acids residues and having from 14 to 34% insulin activity. Disclosed modified C-peptides connecting B30 with A21 are Gln-Pro-Leu-Ala-Leu-Glu-Gly-Ser-Leu-Gln-Lys-Arg and Gly-Tyr-Gly-Ser-Ser-Ser-Arg-Arg-Ala-Pro-Gln-Thr. WO 95/16708, which is specifically incorporated by reference, discloses single-chain insulins with a connecting peptide of 1-15 amino acid residues and with no Lys or Arg as the C-terminal amino acid residue in the connecting peptide. Disclosed modified C-peptide sequences connecting B30 with A21 are Gly-Tyr-Gly-Ser-Ser-Ser-Arg-Arg-Ala-Pro-Gln-Thr and Gly-Tyr-Gly-Ser-Ser-Ser-Ala-Ala-Ala-Pro-Gln-Thr. These single-chain insulins are reported to have insulin activity but also a fairly high affinity to the IGF-1 receptor.

In a further embodiment the single-chain insulin has a connecting peptide selected from the group consisting of VGSSRGKX; VGSSSGX: VGSSSXK; VGSSXGK: VGSXSGK; VGXSSGK; VXSSSGK and XGSSSGK where X is any codable amino acid residue. The following table show selected, non-limiting meanings of X

| | 1. Preference | 2. Preference |
|---|---|---|
| Connecting | X selected from the group | X selected from the group |
| peptide | consisting of | consisting of |
| VGSSRGKX | — | T; L; I; and D |
| VGSSSGX | K and R | P; H; F; T; I; Q; W; A; D; |
| | | and E |
| VGSSSXK | G | A; T; R; and IL |
| VGSSXGK | S; R; A; T; K; P; and N | M; H; Q; V; G; D; and E |
| VGSXSGK | A; R; Y; M; S; and N | G |
| VGXSSGK | T; Q; Y; L; K; R; M; V; H; | G and P |
| | S; and A | |
| VXSSSGK | | |
| XGSSSGK | | |

In one embodiment the single-chain insulin has the formula

B(1-26)-X₁-X₂-X₃-X₄-A(1-21)

wherein X₁ is Thr, Lys or Arg, X₂ is Pro, Lys or Asp, X₃ is Lys, Pro or Glu, and X₄ is a peptide sequence of 6-14 amino acid residues. In another embodiment hereof X₁ is Thr, X₂ is Pro, X₃ is Lys, and X₄ is a peptide sequence of 6-14 amino acid residues. In another embodiment X₄ is 6, 7, 8, 9, 10, 11, 12, 13 or 14 amino acid residues long.

In another embodiment X₄ is selected from the group consisting of Val-Gly-Ser-Ser-Asp-Gly-Lys, Val-Gly-Ser-Ser-Arg-Gly-Lys, Val-Gly-Ser-Ser-Ser-Gly-Lys, Gly-Ser-Ser-Ser-Gly-Lys, Val-Gly-Ser-Ser-Ser-Gly-Lys, Val-Gly-Ser-Ser-Arg-Gly-Lys, Gly-Ser-Ser-Arg-Gly-Lys, Val-Ala-Ser-Ser-Ser-Gly-Lys, and Val-Gly-Ala-Ser-Ser-Gly-Lys; or Val-Gly-Ser-Ala-Ser-Gly-Lys, Val-Gly-Ser-Arg-Ser-Gly-Lys, Val-Gly-Ser-Gly-Ser-Gly-Lys, Val-Gly-Ser-Tyr-Ser-Gly-Lys, Val-Gly-Ser-Met-Ser-Gly-Lys, Val-Gly-Ser-Thr-Ser-Gly-Lys, Val-Gly-Tyr-Ser-Ser-Gly-Lys, Val-Gly-Leu-Ser-Ser-Gly-Lys, Val-Gly-Lys-Ser-Ser-Gly-Lys, Val-Gly-Gly Val-Gly-Tyr-Ser-Ser-Gly-Lys, Val-Gly-Arg-Ser-Ser-Gly-Lys, Val-Gly-Met-Ser-Ser-Gly-Lys, Val-Gly-Val-Ser-Ser-Gly-Lys, and Val-Gly-His-Ser-Ser-Gly-Lys.

In another embodiment the single-chain insulin is selected from the group consisting of B(1-30)-Gly-Ser-Ser-Ser-Gly-Lys-A(1-21); B(1-30)-Val-Gly-Ser-Ser-Ser-Gly-Lys; B(1-30)Val-Gly-Ser-Ser-Arg-Gly-Lys-A(1-21); B(1-30)-Gly-Ser-Ser-Arg-Gly-Lys-A(1-21); B(1-29)-Val-Ala-Gly-Ser-Ser-Ser-Gly-Lys-A(1-21); B(1-29)-Val-Gly-Ala-Ser-Ser-Gly-Lys-A(1-21); B(1-29)Val-Gly-Ser-Ala-Ser-Gly-Lys-A(1-21); B(1-29)-Val-Gly-Ser-Ser-Ala-Gly-Lys-A(1-21); B(1-29)Val-Gly-Ser-Ser-Ser-Gly-Ala-A(1-21); B(1-29)-Val-Gly-Ser-Ser-Ser-Gly-Arg-A(1-21); B(1-29)Val-Gly-Ser-Ser-Ser-Gly-Pro-A(1-21), B(1-29)-Val-Gly-Ser-Ser-Ser-Gly-Gly-A(1-21); B(1-29)Val-Gly-Ser-Ser-Ser-Gly-His-A(1-21); B(1-29)-Val-Gly-Ser-Ser-Ser-Gly-Phe-A(1-21); B(1-29)Val-Gly-Ser-Ser-Ser-Gly-Thr-A(1-21); B(1-29)-Val-Gly-Ser-Ser-Ser-Gly-11e-A(1-21); B(1-29)Val-Gly-Ser-Ser-Ser-Gly-Gln-A(1-21); B(1-29)-Val-Gly-Ser-Ser-Ser-Gly-Trp-A(1-21); B(1-29)Val-Gly-Ser-Ser-Ser-Gly-Phe-A(1-21); B(1-29)-Val-Gly-Ser-Arg-Ser-Gly-Lys-A(1-21); B(1-29)-Val-Gly-Arg-Ser-Ser-Gly-Lys-A(1-21); B(1-29)-Val-Arg-Ser-Ser-Ser-Gly-Lys-A(1-21); B(1-29)-Arg-Gly-Ser-Ser-Ser-Gly-Lys-A(1-21); B(1-29)-Val-Gly-Ser-Ser-Ser-Gly-Lys-A(1-21); B(1-29)-Val-Gly-Arg-Ser-Arg-Gly-Lys-A(1-21); B(1-29)-Val-Gly-Ser-Ser-Arg-Gly-Lys-A(1-21); B(1-29)-Arg-Gly-Ser-Ser-Arg-Gly-Lys-A(1-21); B(1-29)-Val-Gly-Ser-Ser-Arg-Gly-Lys-A(1-21); B(1-29)-Val-Gly-Ser-His-Arg-Gly-Lys-A(1-21); B(1-29)-Val-Gly-His-Ser-Arg-Gly-Lys-A(1-21); B(1-29)-Val-His-Ser-Ser-Arg-Gly-Lys-A(1-21); B(1-29)-His-Gly-Ser-Ser-Arg-Gly-Lys-A(1-21); B(1-30)-Gly-Ser-Ser-Ser-Gly-Arg-A(1-21); B(1-30)-Gly-Arg-Ser-Ser-Gly-Lys-A(1-21); B(1-29)-Val-Gly-Ser-Ala-Ser-Gly-Lys-A (1-21); B(1-29)-Val-Gly-Ser-Asp-Ser-Gly-Lys-A (1-21); B(1-29)Val-Gly-Ser-Gly-Ser-Gly-Lys-A (1-21); B(1-29)-Val-Gly-Ser-Tyr-Ser-Gly-Lys-A (1-21); B(1-29)-Val-Gly-Ser-Met-Ser-Gly-Lys-A (1-21); B(1-29)-Val-Gly-Pro-Ser-Ser-Gly-Lys-A (1-21); B(1-29)-Val-Gly-Thr-Ser-Ser-Gly-Lys-A (1-21); B(1-29)-Val-Gly-Gln-Ser-Ser-Gly-Lys-A (1-21); B(1-29)-Val-Gly-Tyr-Ser-Ser-Gly-Lys-A (1-21); B(1-29)-Val-Gly-Leu-Ser-Ser-Gly-Lys-A (1-21); B(1-29)-Val-Gly-Lys-Ser-Ser-Gly-Lys-A (1-21); B(1-29)-Val-Gly-Gly-Ser-Ser-Gly-Lys-A (1-21); B(1-29)-Val-Gly-Arg-Ser-Ser-Gly-Lys-A (1-21); B(1-29)-Val-Gly-Met-Ser-Ser-Gly-Lys-A (1-21); B(1-29)-Val-Gly-Val-Ser-Ser-Gly-Lys-A (1-21); B(1-29)-Val-Gly-His-Ser-Ser-Gly-Lys-A (1-21); B(1-29)-Leu-Gly-Ser-Ser-Gly-Lys-A(1-21); B(1-29)-Arg-Gly-Ser-Ser-Gly-Lys-A(1-21); B(1-29)-Gln-Gly-Ser-Ser-Gly-Lys-A(1-21); B(1-29)-Gly-Gly-Ser-Ser-Gly-Lys-A(1-21); B(1-29)-Ser-Gly-Ser-Ser-Gly-Lys-A(1-21); B(1-30)-Gly-Ser-Ser-Gly-Lys-A(1-21); B(1-29)-Gln-Gly-Ser-Ser-Gly-Lys-A(1-21); and B(1-29)-Val-Gly-Ser-Ser-Gly-Lys-A(1-21).

In a particular embodiment, the insulin to be employed in any one of the embodiments above is selected from human insulin or insulin compounds selected from the group consisting of:
i. An analogue of human insulin wherein position B28 is Asp, Lys, Leu, Val, or Ala and position B29 is Lys or Pro; and
ii. des(B28-B30), des(B27) or des(B30) human insulin.

In another particular embodiment, the insulin to be employed in any one of the embodiments above is B29-N^{ε}-myristoyl-des(B30) human insulin.

In another embodiment according to any one of the embodiments above the invention relates to a pharmaceutical composition, wherein the insulin peptide is selected from the group consisting of human insulin, B28 Asp human insulin, B28 Lys B29 Pro human insulin, B3 Lys B29 Glu human insulin or B29-N^{ε}-myristoyl-des(B30) human insulin, and the insulinotropic peptide is selected from Arg³⁴ Lys²³(N^{ε}-(γ-Glu(N^{α}-hexadecanoyl)))-GLP-1(7-37), HGEGTFTSDLSKQMEEEAVRLFIEWLKNGGPSSGAPPSKKKKKK-NH2 or exendin-4, and the ligand for the His^{B10} anion site is selected from any one of the embodiments 2 to 204.

In another embodiment according to any one of the embodiments above the invention relates to a pharmaceutical composition, wherein the insulin peptide is selected from the group consisting of human insulin, B28 Asp human insulin, B28 Lys B29 Pro human insulin, or B3 Lys B29 Glu human insulin, and the insulinotropic peptide is selected from Arg³⁴ Lys²⁶(N^{ε}-(γ-Glu(N^{α}-hexadecanoyl)))-GLP-1(7-37), HGEGTFTSDLSKQMEEEAVRLFIEWLKNGGPSSGAPPSKKKKKK-NH2 or exendin-4, and the ligand for the His^{B10} anion site is selected from any one of the embodiments 2 to 204.

In another embodiment according to any one of the embodiments above the invention relates to a pharmaceutical composition, wherein the insulin peptide is selected from the group consisting of human insulin, B28 Asp human insulin, B28 Lys B29 Pro human insulin, B3 Lys B29 Glu human insulin or B29-N^{ε}-myristoyl-des(B30) human insulin, and the insulinotropic peptide is selected from Arg³⁴ Lys²⁶(N^{ε}-(γ-Glu(N^{α}-hexadecanoyl)))-GLP-1(7-37), HGEGTFTSDLSKQMEEEAVRLFIEWLKNGGPSSGAPPSKKKKKK-NH2 or exendin-4, and the ligand for the His^{B10} anion site is the SCN⁻ anion.

In another embodiment according to any one of the embodiments above the invention relates to a pharmaceutical composition, wherein the insulin peptide is selected from the group consisting of human insulin, B28 Asp human insulin, B28 Lys B29 Pro human insulin, B3 Lys B29 Glu human insulin or B29-N-myristoyl-des(B30) human insulin, and the insulinotropic peptide is selected from Arg³⁴ Lys²⁶(N^{ε}-(γ-Glu(N^{α}-hexadecanoyl)))-GLP-1(7-37), HGEGTFTSDLSKQMEEEAVRLFIEWLKNGGPSSGAPPSKKKKKK-NH2 or exendin-4, and the ligand for the His^{B10} anion site is the Cl⁻ anion.

In another embodiment according to any one of the embodiments above the invention relates to a pharmaceutical composition, wherein the insulin peptide is selected from the group consisting of human insulin, B28 Asp human insulin, or B29-N^{ε}-myristoyl-des(B30) human insulin, and the insulinotropic peptide is selected from Arg³⁴ Lys²⁶(N^{ε}-(γ-Glu(N^{α}-hexadecanoyl)))-GLP-1 (7-37), and the ligand for the His^{B10} anion site is selected from any one of the embodiments 2 to 204.

In another embodiment according to any one of the embodiments above the invention relates to a pharmaceutical composition, wherein the insulin peptide is selected from the group consisting of human insulin, B28 Asp human insulin, or B29-N^{ε}-myristoyl-des(B30) human insulin, and the insulinotropic peptide is selected from Arg³⁴ Lys²⁶(N^{ε}-(γ-Glu(N^{α}-hexadecanoyl)))-GLP-1(7-37), and the ligand for the His^{B10} anion site is the SCN⁻ anion.

In another embodiment according to any one of the embodiments above the invention relates to a pharmaceutical composition, wherein the insulin peptide is selected from the group consisting of human insulin, B28 Asp human insulin, or B29-N^{ε}-myristoyl-des(B30) human insulin, and the insulinotropic peptide is selected from Arg³⁴ Lys²⁶(N^{ε}-(γ-Glu(N^{α}-hexadecanoyl)))-GLP-1 (7-37), and the ligand for the His^{B10} anion site is the Cl⁻ anion.

In another embodiment according to any one of the embodiments above the invention relates to a pharmaceutical composition, wherein said insulin peptide is Lys^{B3} Glu^{B29}-human insulin and said insulinotropic peptide is ZP-10 (HGEGTFTSDLSKQMEEEAVRLFIEWLKNGGPSSGAPPSKKKKKK-NH2). In one embodiment according to any one of the embodiments above the concentration of Lys^{B3} Glu^{B29}-human insulin is in the range from about 3.2 mg/mL to about 4.0 mg/mL. In another embodiment according to any one of the embodiments above the concentration of ZP-10 is in the range from about 0.1 mg/mL to about 3 mg/mL.

In another embodiment according to any one of the embodiments above the composition additionally comprises a preservative. In one embodiment, the preservative is phenol, m-cresol or a mixture thereof.

In another embodiment according to any one of the embodiments above the pharmaceutical composition additionally comprises a buffer. In one embodiment according to any one of the embodiments above said buffer is phosphate, TRIS, HEPES, glycine, bicine, diglycine, N-glycylglycine, citrate or mixtures thereof.

In another embodiment according to any one of the embodiments above the pharmaceutical composition additionally comprises an isotonicity agent. In one embodiment according to any one of the embodiments above the isotonicity agent is not a salt. In another embodiment according to any one of the embodiments above the isotonicity agent is selected from mannitol, sorbitol, glycerol, or a mixture thereof.

In another embodiment according to any one of the embodiments above the present invention relates to a soluble pharmaceutical composition which additionally comprises a surfactant. In one embodiment according to any one of the embodiments above the surfactant is selected from a detergent, ethoxylated castor oil, polyglycolyzed glycerides, acetylated monoglycerides, sorbitan fatty acid esters, polysorbate, such as polysorbate-20, poloxamers, such as poloxamer 188 and poloxamer 407, polyoxyethylene sorbitan fatty acid esters, polyoxyethylene derivatives such as alkylated and alkoxylated derivatives (tweens, e.g. Tween-20, or Tween-80), monoglycerides or ethoxylated derivatives thereof, diglycerides or polyoxyethylene derivatives thereof, glycerol, cholic acid or derivatives thereof, lecithins, alcohols and phospholipids, glycerophospholipids (lecithins, kephalins, phosphatidyl serine), glyceroglycolipids (galactopyransoide), sphingophospholipids (sphingomyelin), and sphingoglycolipids (ceramides, gangliosides), DSS (docusate sodium, CAS registry no [577-11-7]), docusate calcium, CAS registry no [128-49-4]), docusate potassium, CAS registry no [7491-09-0]), SDS (sodium dodecyl sulfate or sodium lauryl sulfate), dipalmitoyl phosphatidic acid, sodium caprylate, bile acids and salts thereof and glycine or taurine conjugates, ursodeoxycholic acid, sodium cholate, sodium deoxycholate, sodium taurocholate, sodium glycocholate, N-Hexadecyl-N,N-dimethyl-3-ammonio-1-propanesulfonate, anionic (alkyl-aryl-sulphonates) monovalent surfactants, palmitoyl lysophosphatidyl-L-serine, lysophospholipids (e.g. 1-acyl-sn-glycero-3-phosphate esters of ethanolamine, choline, serine or threonine), alkyl, alkoxyl (alkyl ester), alkoxy (alkyl ether)-derivatives of lysophosphatidyl and phosphatidylcholines, e.g. lauroyl and myristoyl derivatives of lysophosphatidylcholine, dipalmitoylphosphatidylcholine, and modifications of the polar head group, that is cholines, ethanolamines, phosphatidic acid, serines, threonines, glycerol, inositol, and the positively charged DODAC, DOTMA, DCP, BISHOP, lysophosphatidylserine and lysophosphatidylthreonine, zwitterionic surfactants (e.g. N-alkyl-N,N-dimethylammonio-1-propanesulfonates, 3-cholamido-1-propyldimethylammonio-1-propanesulfonate, dodecylphosphocholine, myristoyl lysophosphatidylcholine, hen egg lysolecithin), cationic surfactants (quarternary ammonium bases) (e.g. cetyl-trimethylammonium bromide, cetylpyridinium chloride), non-ionic surfactants, polyethyleneoxide/polypropyleneoxide block copolymers (Pluronics/Tetronics, Triton X-100, Dodecyl β-D-glucopyranoside) or polymeric surfactants (Tween-40, Tween-80, Brij-35), fusidic acid derivatives—(e.g. sodium tauro-dihydrofusidate etc.), long-chain fatty acids and salts thereof C₆-C₁₂ (eg. oleic acid and caprylic acid), acylcarnitines and derivatives, N^{α}-acylated derivatives of lysine, arginine or histidine, or side-chain acylated derivatives of lysine or arginine, N-acylated derivatives of dipeptides comprising any combination of lysine, arginine or histidine and a neutral or acidic amino acid, N^{α}-acylated derivative of a tripeptide comprising any combination of a neutral amino acid and two charged amino acids, or the surfactant may be selected from the group of imidazoline derivatives, or mixtures thereof.

Each one of these specific surfactants constitutes an alternative embodiment of the invention.

In a further embodiment according to any one of the embodiments above the surfactant is a poloxamer, such as poloxamer 188.

In a further embodiment according to any one of the embodiments above the surfactant is a polysorbate, such as polysorbate-20.

In another embodiment according to any one of the embodiments above the invention relates to a pharmaceutical composition, wherein the insulin peptide is selected from the group consisting of human insulin, B28 Asp human insulin, B28 Lys B29 Pro human insulin, B3 Lys B29 Glu human insulin or B29-N^{ε}-myristoyl-des(B30) human insulin, and the insulinotropic peptide is selected from Arg³⁴ Lys²⁶(N^{ε}-(γ-Glu(N^{α}-hexadecanoyl)))-GLP-1(7-37), HGEGTFTSDLSKQMEEEAVRLFIEWLKNGGPSSGAPPSKKKKKK-NH2 or exendin-4, and the ligand for the His^{B10} anion site is selected from any one of the embodiments 2 to 204 and the surfactant selected from a polysorbate or poloxamer.

In another embodiment according to any one of the embodiments above the invention relates to a pharmaceutical composition, wherein the insulin peptide is selected from the group consisting of human insulin, B28 Asp human insulin, B28 Lys B29 Pro human insulin, B3 Lys B29 Glu human insulin or B29-N^{ε}-myristoyl-des(B30) human insulin, and the insulinotropic peptide is selected from Arg³⁴ Lys²⁶(N^{ε}-(γ-Glu(N^{α}-hexadecanoyl)))-GLP-1(7-37), HGEGTFTSDLSKQMEEEAVRLFIEWLKNGGPSSGAPPSKKKKKK-NH2 or exendin-4, and the ligand for the His^{B10} anion site is the SCN⁻ anion, and the surfactant is selected from a polysorbate or poloxamer.

In another embodiment according to any one of the embodiments above the invention relates to a pharmaceutical composition, wherein the insulin peptide is selected from the group consisting of human insulin, B28 Asp human insulin, B28 Lys B29 Pro human insulin, B3 Lys B29 Glu human insulin or B29-N^{ε}-myristoyl-des(B30) human insulin, and the insulinotropic peptide is selected from Arg³⁴ Lys²⁶(N^{ε}-(γ-Glu(N^{α}-hexadecanoyl)))-GLP-1(7-37), HGEGTFTSDLSKQMEEEAVRLFIEWLKNGGPSSGAPPSKKKKKK-NH2 or exendin-4, and the ligand for the His^{B10} anion site is the Cl⁻ anion, and the surfactant is selected from a polysorbate or poloxamer.

In another embodiment according to any one of the embodiments above the invention relates to a pharmaceutical composition, wherein the insulin peptide is selected from the group consisting of human insulin, B28 Asp human insulin, or B29-N^{ε}-myristoyl-des(B30) human insulin, and the insulinotropic peptide is selected from Arg³⁴ Lys²⁶(N^{ε}-(γ-Glu(N^{α}-hexadecanoyl)))-GLP-1(7-37), and the ligand for the His^{B10} anion site is selected from any one of the embodiments 2 to 204 and the surfactant selected from a polysorbate or poloxamer.

In another embodiment according to any one of the embodiments above the invention relates to a pharmaceutical composition, wherein the insulin peptide is selected from the group consisting of human insulin, B28 Asp human insulin, or B29-N^{ε}-myristoyl-des(B30) human insulin, and the insulinotropic peptide is selected from Arg³⁴ Lys²⁶(N^{ε}-(γ-Glu(N^{α}-hexadecanoyl)))-GLP-1(7-37), and the ligand for the His^{B10} anion site is the SCN⁻ anion, and the surfactant is selected from a polysorbate or poloxamer.

In another embodiment according to any one of the embodiments above the invention relates to a pharmaceutical composition, wherein the insulin peptide is selected from the group consisting of human insulin, B28 Asp human insulin, or B29-N^{ε}-myristoyl-des(B30) human insulin, and the insulinotropic peptide is selected from Arg³⁴ Lys²⁶(N-(γ-Glu(N^{α}-hexadecanoyl)))-GLP-1 (7-37), and the ligand for the His^{B10} anion site is the Cl⁻ anion, and the surfactant is selected from a polysorbate or poloxamer.

In another embodiment the invention provides a pharmaceutical composition comprising 0-6 moles zinc²⁺ ions per mole insulin hexamer.

In another embodiment the invention provides a pharmaceutical composition comprising 2-3 moles zinc²⁺ ions per mole insulin hexamer.

In another embodiment the invention provides a pharmaceutical composition comprising 3-6 moles zinc²⁺ ions per mole insulin hexamer.

In one embodiment of the invention the concentration of added ligand for the zinc site is between 0.2 and 39 times the minimum of either [Zn²⁺ ] or ⅓*[insulin].

In one embodiment of the invention the concentration of added ligand for the zinc site is between 0.2 and 10 times the minimum of either [Zn²⁺] or ⅓*[insulin].

The insulin composition of the present invention may have a pH value in the range of 6.5 to 9, e.g. 7 to 8.5, or in the range of 7.4 to 8.2.

In another aspect the present invention relates to a method for treatment of hyperglycemia comprising parenteral administration of an effective amount of a pharmaceutical composition according to any one of the embodiments above. When the pharmaceutical compositions according to any one of the embodiments above are administered by a pump, it is typically administered continuously or discontinuously via at least 10 administrations or more per day. In one aspect the method of treatment comprises administration of an effective amount of the pharmaceutical composition according to any one of the embodiments above which is from about 30 μL/day to about 600 μL/day, such as from about 60 μL/day to about 360 μL/day. In another embodiment hereof the method comprises a pharmaceutical composition according to any one of the embodiments above for administration by subcutaneous injection.

In another embodiment the method comprises a pharmaceutical composition according to any one of the embodiments above for administration by a pump.

In another embodiment the method comprises administration by a pump which delivers a discontinuous amount of said pharmaceutical composition according to any one of the embodiments above.

In another embodiment the method comprises administration by a pump which delivers a discontinuous amount of said pharmaceutical composition according to any one of the embodiments above wherein said discontinuous administration of said pharmaceutical composition is by a pulse dosing for a period of time which is less than the period between pulses.

### Combination Treatment

In a further aspect of the present invention the compositions of the present invention may be administered in combination with one or more further active substances in any suitable ratios. Such further active agents may be selected from antidiabetic agents, antihyperlipidemic agents, antiobesity agents, antihypertensive agents and agents for the treatment of complications resulting from or associated with diabetes.

Suitable antidiabetic agents include orally active hypoglycemic agents.

Suitable orally active hypoglycemic agents preferably include imidazolines, sulfonylureas, biguanides, meglitinides, oxadiazolidinediones, thiazolidinediones, insulin sensitizers, α-glucosidase inhibitors, agents acting on the ATP-dependent potassium channel of the pancreatic β-cells eg potassium channel openers such as those disclosed in WO 97/26265, WO 99/03861 and WO 00/37474 (Novo Nordisk A/S) which are incorporated herein by reference, potassium channel openers, such as ormitiglinide, potassium channel blockers such as nateglinide or BTS-67582, glucagon antagonists such as those disclosed in WO 99/01423 and WO 00/39088 (Novo Nordisk A/S and Agouron Pharmaceuticals, Inc.), all of which are incorporated herein by reference, GLP-1 agonists such as those disclosed in WO 00/42026 (Novo Nordisk A/S and Agouron Pharmaceuticals, Inc.), which are incorporated herein by reference, DPP-IV (dipeptidyl peptidase-IV) inhibitors, PTPase (protein tyrosine phosphatase) inhibitors, inhibitors of hepatic enzymes involved in stimulation of gluconeogenesis and/or glycogenolysis, glucose uptake modulators, GSK-3 (glycogen synthase kinase-3) inhibitors, compounds modifying the lipid metabolism such as antihyperlipidemic agents and antilipidemic agents, compounds lowering food intake, and PPAR (peroxisome proliferator-activated receptor) and RXR (retinoid X receptor) agonists such as ALRT-268, LG-1268 or LG-1069.

In one embodiment of the present invention, the compositions of the present invention may be administered in combination with a sulphonylurea eg tolbutamide, chlorpropamide, tolazamide, glibenclamide, glipizide, glimepiride, glicazide or glyburide.

In one embodiment of the present invention, the compositions of the present invention may be administered in combination with a biguanide eg metformin.

In one embodiment of the present invention, the compositions of the present invention may be administered in combination with a meglitinide eg repaglinide or senaglinide/nateglinide.

In one embodiment of the present invention, the compositions of the present invention may be administered in combination with a thiazolidinedione insulin sensitizer eg troglitazone, ciglitazone, pioglitazone, rosiglitazone, isaglitazone, darglitazone, englitazone, CS-011/CI-1037 or T 174 or the compounds disclosed in WO 97/41097 (DRF-2344), WO 97/41119, WO 97/41120, WO 00/41121 and WO 98/45292 (Dr. Reddy's Research Foundation), which are incorporated herein by reference.

In one embodiment of the present invention the compositions of the present invention may be administered in combination with an insulin sensitizer eg such as GI 262570, YM-440, MCC-555, JTT-501, AR-H039242, KRP-297, GW-409544, CRE-16336, AR-H049020, LY510929, MBX-102, CLX-0940, GW-501516 or the compounds disclosed in WO 99/19313 (NN622/DRF-2725), WO 00/50414, WO 00/63191, WO 00/63192, WO 00/63193 (Dr. Reddy's Research Foundation) and WO 00/23425, WO 00/23415, WO 00/23451, WO 00/23445, WO 00/23417, WO 00/23416, WO 00/63153, WO 00/63196, WO 00/63209, WO 00/63190 and WO 00/63189 (Novo Nordisk A/S), which are incorporated herein by reference.

In one embodiment of the present invention the compositions of the present invention may be administered in combination with an α-glucosidase inhibitor eg voglibose, emiglitate, miglitol or acarbose.

In one embodiment of the present invention the compositions of the present invention may be administered in combination with a glycogen phosphorylase inhibitor eg the compounds described in WO 97/09040 (Novo Nordisk A/S).

In one embodiment of the present invention the compositions of the present invention may be administered in combination with an agent acting on the ATP-dependent potassium channel of the pancreatic β-cells eg tolbutamide, glibenclamide, glipizide, glicazide, BTS-67582 or repaglinide.

In one embodiment of the present invention the compositions of the present invention may be administered in combination with nateglinide.

In one embodiment of the present invention the compositions of the present invention may be administered in combination with an antihyperlipidemic agent or a antilipidemic agent eg cholestyramine, colestipol, clofibrate, gemfibrozil, lovastatin, pravastatin, simvastatin, probucol or dextrothyroxine.

Furthermore, the compositions of the present invention may be administered in combination with one or more antiobesity agents or appetite regulating agents.

Such agents may be selected from the group consisting of CART (cocaine amphetamine regulated transcript) agonists, NPY (neuropeptide Y) antagonists, MC3 (melanocortin 3) agonists, MC4 (melanocortin 4) agonists, orexin antagonists, TNF (tumor necrosis factor) agonists, CRF (corticotropin releasing factor) agonists, CRF BP (corticotropin releasing factor binding protein) antagonists, urocortin agonists, P3 adrenergic agonists such as CL-316243, AJ-9677, GW-0604, LY362884, LY377267 or AZ-40140, MSH (melanocytestimulating hormone) agonists, MCH (melanocyte-concentrating hormone) antagonists, CCK (cholecystokinin) agonists, serotonin reuptake inhibitors (fluoxetine, seroxat or citalopram), serotonin and norepinephrine reuptake inhibitors, 5HT (serotonin) agonists, bombesin agonists, galanin antagonists, growth hormone, growth factors such as prolactin or placental lactogen, growth hormone releasing compounds, TRH (thyreotropin releasing hormone) agonists, UCP 2 or 3 (uncoupling protein 2 or 3) modulators, leptin agonists, DA (dopamine) agonists (bromocriptin, doprexin), lipase/amylase inhibitors, PPAR modulators, RXR modulators, TR β agonists, adrenergic CNS stimulating agents, AGRP (agouti related protein) inhibitors, H3 histamine antagonists such as those disclosed in WO 00/42023, WO 00/63208 and WO 00/64884, which are incorporated herein by reference, exendin-4, GLP-1 agonists, ciliary neurotrophic factor, and oxyntomodulin. Further antiobesity agents are bupropion (antidepressant), topiramate (anticonvulsant), ecopipam (dopamine D1/D5 antagonist) and naltrexone (opioid antagonist).

In one embodiment of the present invention the antiobesity agent is leptin.

In one embodiment of the present invention the antiobesity agent is a serotonin and norepinephrine reuptake inhibitor eg sibutramine.

In one embodiment of the present invention the antiobesity agent is a lipase inhibitor eg orlistat.

In one embodiment of the present invention the antiobesity agent is an adrenergic CNS stimulating agent eg dexamphetamine, amphetamine, phentermine, mazindol phendimetrazine, diethylpropion, fenfluramine or dexfenfluramine.

Furthermore, compositions of the present invention may be administered in combination with one or more antihypertensive agents. Examples of antihypertensive agents are β-blockers such as alprenolol, atenolol, timolol, pindolol, propranolol and metoprolol, ACE (angiotensin converting enzyme) inhibitors such as benazepril, captopril, enalapril, fosinopril, lisinopril, quinapril and ramipril, calcium channel blockers such as nifedipine, felodipine, nicardipine, isradipine, nimodipine, diltiazem and verapamil, and α-blockers such as doxazosin, urapidil, prazosin and terazosin. Further reference can be made to Remington: The Science and Practice of Pharmacy, 19th Edition, Gennaro, Ed., Mack Publishing Co., Easton, Pa., 1995.

In another aspect of the present invention, the compositions of the present invention may be administered in combination with more than one of the above-mentioned compounds e.g. in combination with metformin and a sulphonylurea such as glyburide; a sulphonylurea and acarbose; nateglinide and metformin; acarbose and metformin; a sulfonylurea, metformin and troglitazone.

It should be understood that any suitable combination of the compositions of the present invention with diet and/or exercise, one or more of the above-mentioned compounds and optionally one or more other active substances are considered to be within the scope of the present invention.

### EXAMPLES

### Example 1

Insulin Aspart (B28 Asp human insulin) and liraglutide mixtures were prepared with constant aspart concentration (0.6 mM) and increasing concentration of liraglutide (going from 0 to 2.4 mM). Mixtures are formulated at pH 7.5, 1.6% w/v d-glycerol, 0.3 mM Zn(Ac)₂, 30 mM d-phenol (deuterated phenol in order to avoid any proton NMR signal). The proton NMR spectra for each mix ratio were recorded and displayed showing the region between 5 and 6 ppm. This region of the proton NMR signal only included signals from aspart, no signals from liraglutide were present.

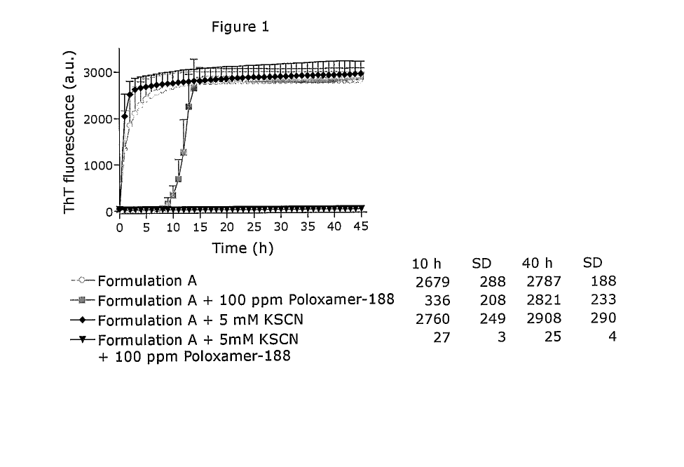

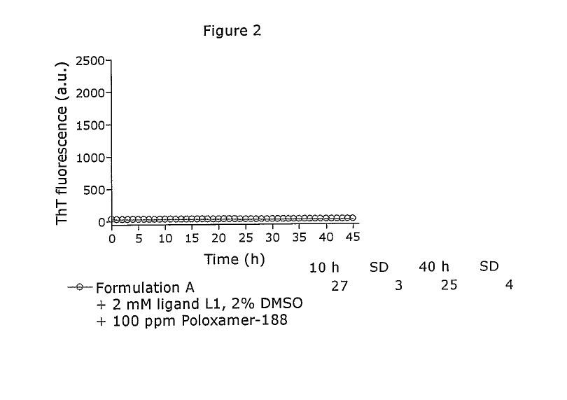

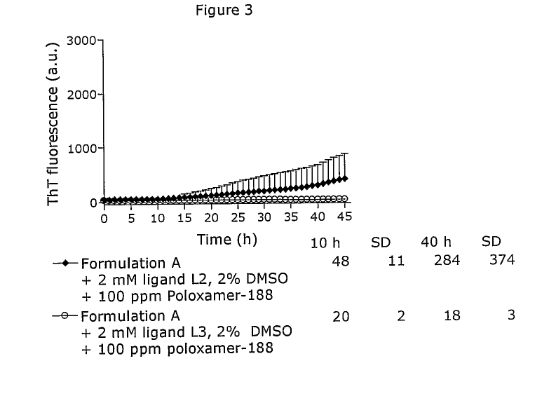

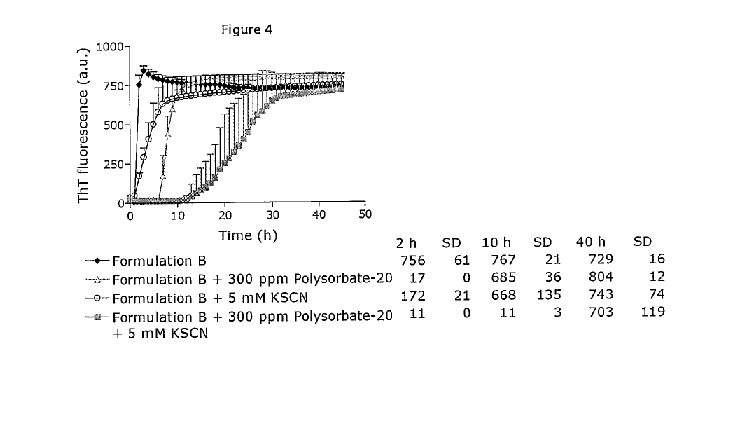

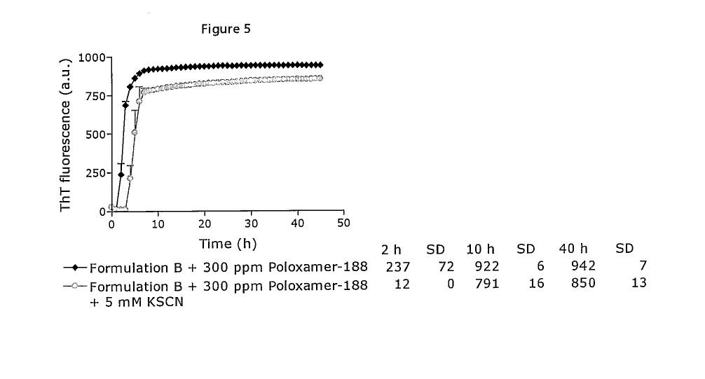

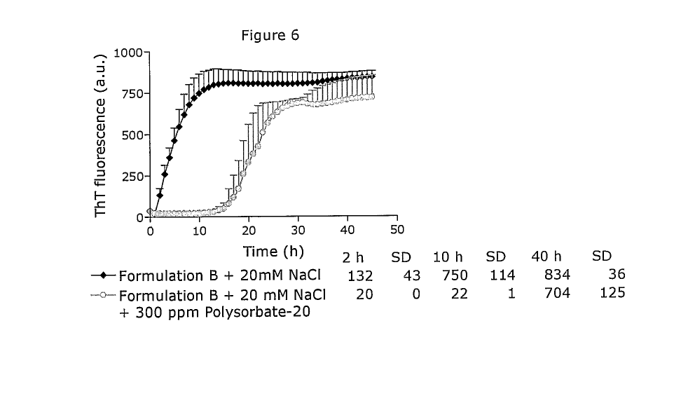

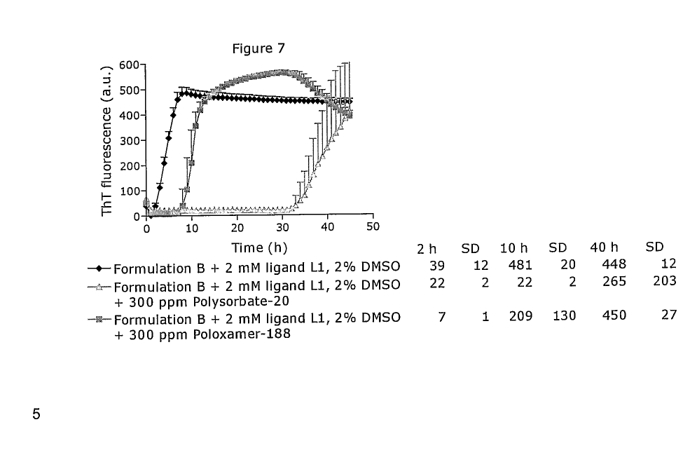

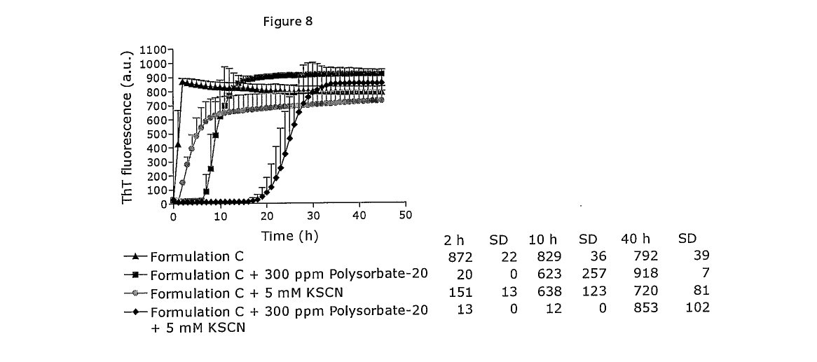

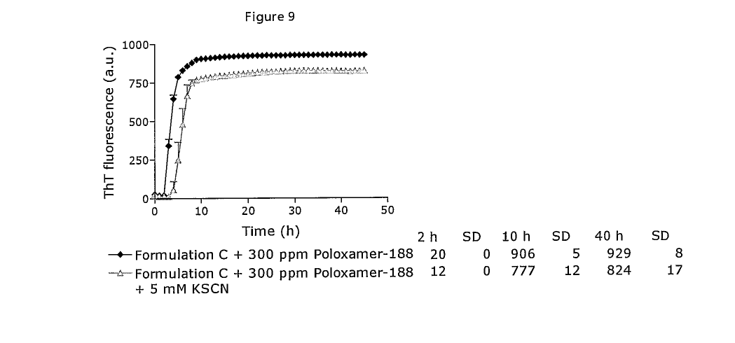

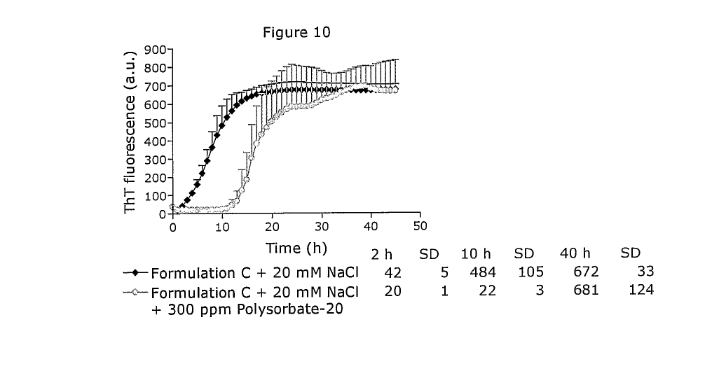

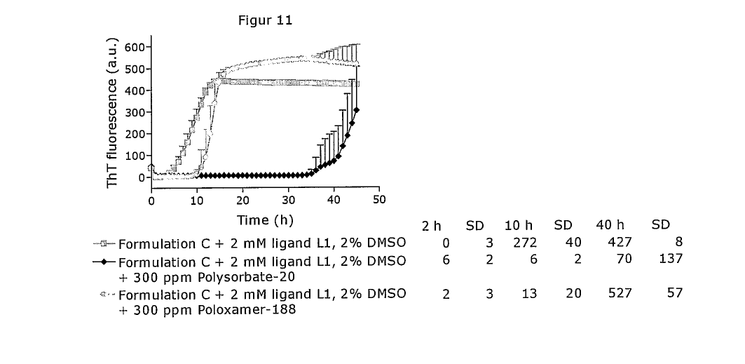

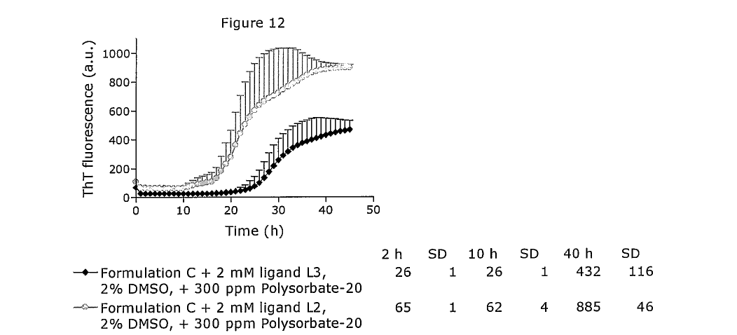

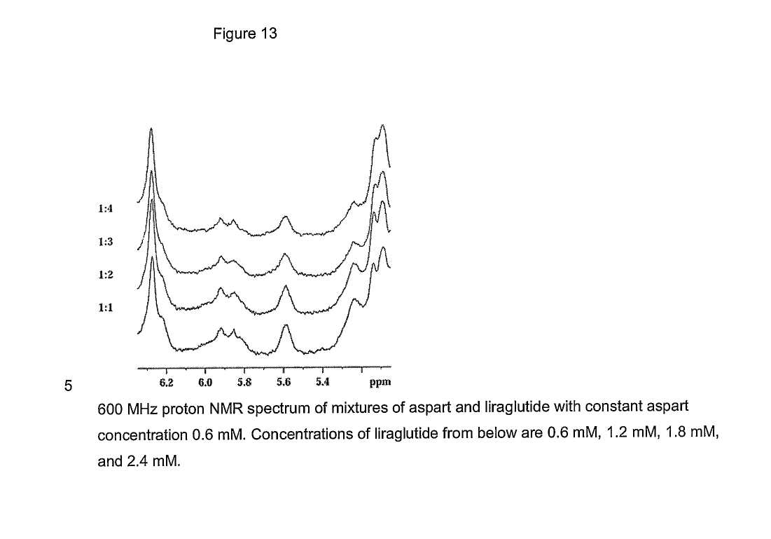

FIG. 13 shows that as the concentration of liraglutide increased the signal at 5.6 ppm (the alpha proton of Cys A6) decreased and lost intensity. This particular signal from Cys A6 indicates that aspart is in the hexameric form designated R₆. As liraglutide concentration increased in the mixtures, the concentration of aspart R₆ hexamers was decreased, and instead less stable forms of aspart were present.

Ligands for the His^{B10} anion site of the R₆ hexameric aspart entity will stabilize this R₆ conformation. Two such ligands were tested under the same conditions as was used in the experiment depicted in FIG. 13 with the addition of ligands at 3 mM concentration. NMR spectra of mixtures with added ligand displayed a significantly increased intensity of the resonance of the alpha proton of Cys A6 (at 5.6 ppm) and generally several signals between 5 and 6 ppm. Several of these signals were resonances from the R₆ hexameric unit showing that this form of aspart has been significantly stabilized.

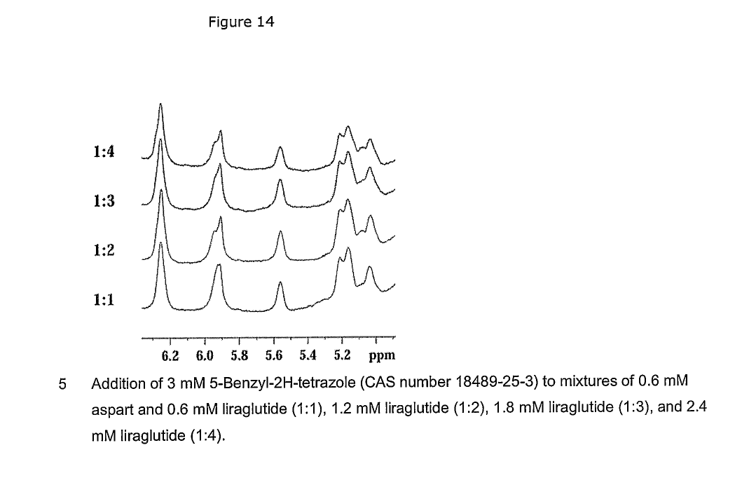

FIG. 14 shows spectra corresponding to those in FIG. 13 of the same mixtures except for the addition of 5-Benzyl-2H-tetrazole.

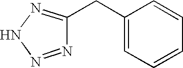

### 5-Benzyl-2H-tetrazole

The presence of a ligand for the His^{B10} anion site surprisingly narrowed the resonance lines (stabilized the R₆ conformation) and increased the signal intensity showing that the general content of the R₆ conformers has increased dramatically.

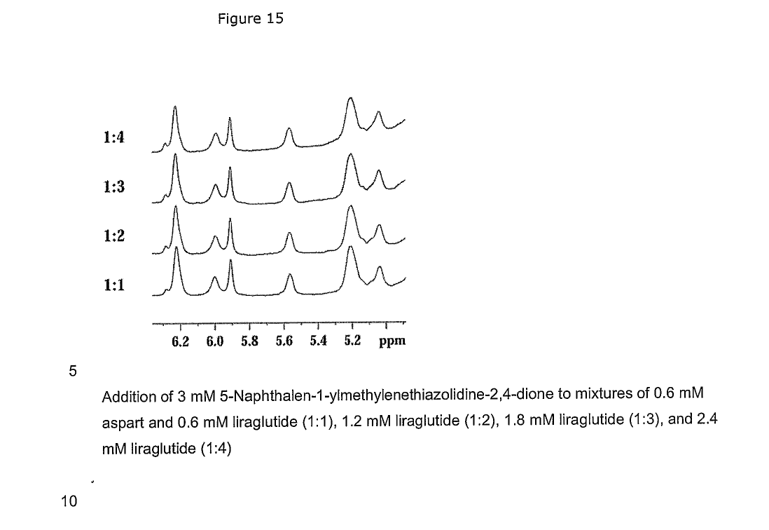

FIG. 15 shows spectra corresponding to FIG. 14, except that a different ligand has been used, 5-Naphthalen-1-ylmethylenethiazolidine-2,4-dione:

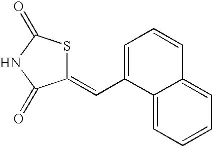

The present ligand binds even tighter than the 5-Benzyl-2H-tetrazole used in the experiments shown in FIG. 14. The invariant aspart resonances under increasing liraglutide concentration demonstrate the ability of such ligands to bind to the Zn-binding pocket of aspart.

### Example 2

Mixtures of human insulin and liraglutide at pH 7.4, 1.6 w/v glycerol, 0.3 mM Zinc acetate with the following concentrations were prepared for evaluation of storage stability:

| | | | |
|---|---|---|---|
| Sample number | Human insulin | Liraglutide | Observation^{§} |

| | | | | | |
|---|---|---|---|---|---|
| 1 | 0 | mM | 6.0 | mM | 15 |
| 2 | 1.2 | mM | 0 | mM | — |
| 3 | 0.6 | mM | 0.6 | mM | — |
| 4 | 0.6 | mM | 1.2 | mM | — |
| 5 | 0.6 | mM | 1.8 | mM | 48 |
| 6 | 0.6 | mM | 2.4 | mM | 17 |
| 7 | 0.6 | mM | 3.0 | mM | 17 |

^{§}Precipitation, cloudiness or other sources of non transparency of samples were visually evaluated while the samples were stored at room temperature (22 degrees Celcius) for two months. The reported numbers are the number of days after the sample preparation when precipitation, cloudiness or other sources of non transparency of samples were observed.

Samples with high content of liraglutide have a poor physical stability.

### Example 3

Mixtures of aspart and liraglutide at pH 7.4, 1.6 w/v glycerol, 0.3 mM Zinc acetate with the following concentrations were prepared for evaluation of storage stability

| Sample number | aspart | Liraglutide | Observation^{§} |
|---|---|---|---|
| 1 | 0.6 mM | 0.6 mM | — |
| 2 | 0.6 mM | 1.2 mM | — |
| 3 | 0.6 mM | 1.8 mM | — |
| 4 | 0.6 mM | 2.4 mM | — |
| 5 | 0.6 mM | 3.0 mM | 17 |

^{§}Precipitation, cloudiness or other sources of non transparency of samples were visually evaluated while the samples were stored at room temperature (22 degrees Celcius) for two months. The reported numbers are the number of days after the sample preparation when precipitation, cloudiness or other sources of non transparency of samples were observed.

All samples remained transparent throughout the two month storage period.

### Example 4

Mixtures of aspart and liraglutide at pH 7.9, 1.6 w/v glycerol, 0.3 mM Zinc acetate, and 3.0 mM 5-Benzyl-2H-tetrazole with the following concentrations were prepared for evaluation of storage stability.

| Sample number | aspart | Liraglutide | Observation^{§} |
|---|---|---|---|
| 1 | 0.6 mM | 0.6 mM | — |
| 2 | 0.6 mM | 1.2 mM | — |
| 3 | 0.6 mM | 1.8 mM | — |
| 4 | 0.6 mM | 2.4 mM | — |
| 5 | 0.6 mM | 3.0 mM | — |

^{§}Precipitation, cloudiness or other sources of non transparency of samples were visually evaluated while the samples were stored at room temperature (22 degrees Celcius) for two months. The reported numbers are the number of days after the sample preparation when precipitation, cloudiness or other sources of non transparency of samples were observed.

All samples remained transparent throughout the two month storage period when stabilized with 3.0 mM 5-Benzyl-2H-tetrazole.

### Example 5

Mixtures of aspart and liraglutide at pH 7.9, 1.6 w/v glycerol, 0.3 mM Zinc acetate, and a ligand as specified in the table with aspart at 0.6 mM concentration and liraglutide 1.2 mM were prepared for evaluation of storage stability.

| Concentration | | Ligand |
|---|---|---|
| 0.35 | mM | 7-bromo-3-hydroxy-2-naphthoic acid |
| 1.0 | mM | 7-bromo-3-hydroxy-2-naphthoic acid |
| 0.35 | mM | 4-[3-(1h-tetrazol-5-yl)carbazol-9-ylmethyl]benzoic acid |
| 1.0 | mM | 4-[3-(1h-tetrazol-5-yl)carbazol-9-ylmethyl]benzoic acid |
| 0.35 | mM | [4-(2,4-dioxothiazolidin-5-ylidenemethyl)naphthalen-1- |
| | | yloxy]acetic acid |
| 1.0 | mM | [4-(2,4-dioxothiazolidin-5-ylidenemethyl)naphthalen-1- |
| | | yloxy]acetic acid |

All ligands have high affinity to the Zn-binding pocket of human insulin and aspart.

All samples remained transparent for the two month storage period.

### Example 6

FIGS. 1-12 show the physical stability (i.e. the tendency to fibrillate) for three different mixes of an insulin and liraglutide with various additions of surfactants or/and a ligand for the His^{B10} anion sites.

Formulation A (shown in FIGS. 1-3) consists of: 1.2 mM liraglutide (Arg³⁴ Lys²⁶(N^{ε}-(γ-Glu(N^{α}-hexadecanoyl)))-GLP-1(7-37)), 0.6 mM insulin aspart (B28 Asp human insulin), 0.2 mM Zn²⁺ (corresponding to 2 Zn²⁺ ions per insulin hexamer),14 mg/ml propylene glycol, 60 mM phenol, 5 mM phosphate, pH 7.7.

Formulation B (shown in FIGS. 4-7) consists of: 2.4 mM insulin detemir (B29-N^{ε}-myristoyl-des(B30) human insulin), 1.6 mM Zn²⁺ (corresponding to 4 Zn²⁺ ions/insulin hexamer), 1.2 mM liraglutide, 14 mg/ml propylene glycol, 60 mM phenol, 5 mM phosphate, pH 7.7.

Formulation C (shown in FIGS. 8-12) consists of: 2.4 mM insulin detemir, 2.0 mM Zn²⁺ (corresponding to 5 Zn²⁺ ions/insulin hexamer), 1.2 mM liraglutide, 14 mg/ml propylene glycol, 60 mM phenol, 5 mM phosphate, pH 7.7.

Formulation A fibrillates almost instantaneously, see FIG. 1. The addition of 100 ppm Poloxamer-188 introduces a lag time of approximately 10 hours, hence increasing the physical stability of formulation A. The addition of 5 mM KSCN (of which the SCN⁻ anion is a ligand for the His^{B10} anion sites) does not increase the stability of formulation A. However, adding both 100 ppm Poloxamer-188 and 5 mM KSCN to formulation A increases the stability significantly and no fibrillation at all is observed during the assay time of 45 hours, see FIG. 1. This stability is significantly higher than the stabilising effect obtained by adding 100 ppm Poloxamer-188 alone. Hence, a surprising significantly increased stability occurs when adding both a surfactant and a ligand for the His^{B10} anion sites to formulation A.

Likewise, FIG. 2 shows that adding a ligand L1, the compound of Example 533, which can be seen in WO 2004/056347 (Novo Nordisk), page 226, which is hereby incorporated by reference, to formulation A with 100 ppm Poloxamer-188 increases the stability further compared to formulation A with 100 ppm Poloxamer-188 as shown in FIG. 1. The ligand L1 is added from an appropriate stock solution in DMSO, resulting in a final DMSO concentration of 2% in the sample.

In FIG. 3 the addition of both 100 ppm Poloxamer-188 and 2 mM of a ligand L2, the compound of Example 284, which can be seen in WO 2004/056347 (Novo Nordisk), page 171-172, which is hereby incorporated by reference, to Formulation A results in a lag time of approximately 15 hours, compared to the lag time of approx. 10 hours for formulation A with 100 ppm Poloxamer-188 alone as shown in FIG. 1. Furthermore, the fibrillation rate for the sample with both Poloxamer-188 and the ligand L2, is much slower than the sample with Poloxamer-188 alone. FIG. 3 also shows that the addition of both 100 ppm Poloxamer-188 and 2 mM of a ligand L3, the compound of Example 283, which can be seen in WO 2004/056347 (Novo Nordisk), page 171, which is hereby incorporated by reference, prolongs the lag time for fibrillation to more than 45 hours compared to the lag time of approx. 10 hours for formulation A with only Poloxamer-188 as shown in FIG. 1.

FIG. 4 shows that formulation B starts to fibrillate after a very short lag time of approx. 1 hour. Adding 300 ppm Polysorbate-20 increases the lag time to approx. 5 hours. The addition of 5 mM KSCN to formulation B slows the fibrillation rate. However, the addition of both 300 ppm Polysorbate-20 and 5 mM KSCN to formulation B prolongs the lag time to approx. 10 hours. Again, a surprising synergistic effect stabilising formulation B is obtained by adding both Polysorbate-20 and KSCN.

FIG. 5 shows the fibrillation tendency of formulation B with 300 ppm Poloxamer-188 added. When further adding 5 mM KSCN, the lag time is approx. 3 hours, which should be compared with the lag time of 1 hour for formulation B with 5 mM KSCN alone as shown in FIG. 4.

FIG. 6 shows that adding 20 mM NaCl (of which the Cl⁻ anion is a ligand for the His^{B10} anion sites) to formulation B reduces the fibrillation rate. Adding both 20 mM NaCl and 300 ppm Polysorbate-20 results in a lag time of approx. 15 hours, longer than the lag time obtained by adding Polysorbate-20 alone to formulation B as shown in FIG. 4.

FIG. 7 shows that adding both 300 ppm Polysorbate-20 and 2 mM of the ligand L1 to formulation B results in a lag time of more than 30 hours, significantly longer than that obtained by adding either 300 ppm Polysorbate-20 alone (lag time approx. 5 hours, see FIG. 4) or the ligand L1 alone (see FIG. 7). Likewise, the combination of both 300 ppm Poloxamer-188 and 2 mM of the ligand L1 results in a longer lag time (approx. 8 hours) than obtained by addition of either Poloxamer-188 (see FIG. 5) or the ligand L1 (see FIG. 7) to formulation B.

The stability of formulation C is shown in FIG. 8. It fibrillates almost instantaneously. Adding 300 ppm Polysorbate-20 increases the lag time to approx. 6 hours. Adding 5 mM KSCN to formulation C increases the lag time and reduces the fibrillation rate. As observed with the previous two formulations, the addition of both 300 ppm Polysorbate-20 and 5 mM KSCN results in a surprisingly long lag time of approx. 20 hours, see FIG. 8.

The addition of 300 ppm Poloxamer-188 to formulation C increases the lag time to approx. 2 hours. Further addition of 5 mM KSCN results in an even longer lag time, see FIG. 9. This lag time is also longer than with KSCN alone, compare with FIG. 8.

FIG. 10 shows that the addition of 20 mM NaCl to formulation C increases the lag time and slows the fibrillation rate compared to FIG. 8. Adding both 20 mM NaCl and 300 ppm Polysorbate-20 to formulation C results in lag time of more than 10 hours, which is longer than the 6 hours observed with Polysorbate-20 alone, see FIG. 8.

Adding 2 mM of the ligand L1, to formulation C increases the lag time to approx. 4 hours. Adding both the ligand L1, and 300 ppm Polysorbate-20 prolongs the lag time to more than 30 hours, see FIG. 11. This is a significant prolongation compared to the lag time of approx. 6 hours with Polysorbate-20 alone as shown in FIG. 8. Adding 300 ppm Poloxamer-188 to Formulation C with 2 mM of the ligand L1 the resulting lag time of approx. 10 hours is longer than with either Poloxamer-188 (see FIG. 9) or the ligand L1 (see FIG. 11) alone.

Similar results are obtained using the ligands L2 and L3 (see FIG. 12): In their presence the lag times are prolonged compared to formulation C with 300 ppm Polysorbate-20 alone (see FIG. 8).

1. A pharmaceutical composition comprising an insulinotropic peptide, an insulin peptide and a ligand for the His^{B10} anion site selected from the group consisting of carboxylates, dithiocarboxylates, phenolates, thiophenolates, alkylthiolates, sulfonamides, imidazoles, triazoles, 4-cyano-1,2,3-triazoles, pyrimidine-2,4,6-triones, benzimidazoles, benzotriazoles, purines, thiazolidinediones, tetrazoles, 5-mercaptotetrazoles, rhodanines, N-hydroxyazoles, hydantoines, thiohydantoines, barbiturates, naphthoic acids, salicylic acids, salts containing SCN⁻ anions and salts containing Cl⁻ anions.

2. The pharmaceutical composition according to claim 1 comprising an insulinotropic peptide, an insulin peptide and a ligand for the His^{B10} anion site selected from the group consisting of carboxylates, dithiocarboxylates, phenolates, thiophenolates, alkylthiolates, sulfonamides, imidazoles, triazoles, 4-cyano-1,2,3-triazoles, pyrimidine-2,4,6-triones, benzimidazoles, benzotriazoles, purines, thiazolidinediones, tetrazoles, 5-mercaptotetrazoles, rhodanines, N-hydroxyazoles, hydantoines, thiohydantoines, barbiturates, naphthoic acids and salicylic acids.

3. The pharmaceutical composition according to claim 1 comprising an insulinotropic peptide, an insulin peptide, a ligand for the His^{B10} anion site selected from the group consisting of carboxylates, dithiocarboxylates, phenolates, thiophenolates, alkylthiolates, sulfonamides, imidazoles, triazoles, 4-cyano-1,2,3-triazoles, pyrimidine-2,4,6-triones, benzimidazoles, benzotriazoles, purines, thiazolidinediones, tetrazoles, 5-mercaptotetrazoles, rhodanines, N-hydroxyazoles, hydantoines, thiohydantoines, barbiturates, naphthoic acids, salicylic acids, salts containing SCN⁻ anions and salts containing Cl⁻ anions, and optionally one or more of a surfactant.

4. The pharmaceutical composition according to claim 1 wherein the ligand for the His^{B10} anion site is 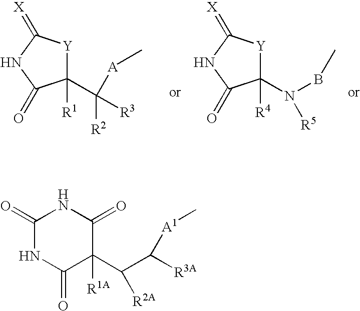
wherein
X is ═O, ═S or ═NH
Y is —S—, —O— or —NH—
R¹, R^{1A} and R⁴ are independently selected from hydrogen or C₁-C₆-alkyl,
R² and R^{2A} are hydrogen or C₁-C₆-alkyl or aryl, R¹ and R² may optionally be combined to form a double bond, R^{1A} and R^{2A} may optionally be combined to form a double bond,
R³, R^{3A} and R⁵ are independently selected from hydrogen, halogen, aryl optionally substituted with one or more substituents independently selected from R¹⁶, C₁-C₆-alkyl, or —C(O)NR¹¹R¹²,
A, A¹ and B are independently selected from C₁-C₆-alkyl, aryl, aryl-C₁-C₆-alkyl, —NR¹¹-aryl, aryl-C₂-C₆-alkenyl or heteroaryl, wherein the alkyl or alkenyl is optionally substituted with one or more substituents independently selected from R⁶ and the aryl or heteroaryl is optionally substituted with up to four substituents R⁷, R⁸, R⁹, and R¹⁰,
A and R³ may be connected through one or two valence bonds, B and R⁵ may be connected through one or two valence bonds,
R⁶ is independently selected from halogen, —CN, —CF₃, —OCF₃, aryl, —COOH and —NH₂,
R⁷, R⁸, R⁹ and R¹⁰ are independently selected from
hydrogen, halogen, —CN, —CH₂CN, —CHF₂, —CF₃, —OCF₃, —OCHF₂, —OCH₂CF₃, —OCF₂CHF₂, —S(O)₂CF₃, —OS(O)₂CF₃, —SCF₃, —NO₂, —OR¹¹, —NR¹¹R¹², —SR¹¹, —NR¹¹S(O)₂R¹², —S(O)₂NR¹¹R¹², —S(O)NR¹¹R¹², —S(O)R¹¹, —S(O)₂R¹¹, —OS(O)₂R¹¹, —C(O)NR¹¹R¹², —OC(O)NR¹¹R¹², —NR¹¹C(O)R¹², —CH₂C(O)NR¹¹R¹², —OC₁-C₆-alkyl-C(O)NR¹¹R¹², —CH₂OR¹¹, —CH₂OC(O)R¹¹, —CH₂NR¹¹R¹², —OC(O)R¹¹, —OC₁-C₁₅-alkyl-C(O)OR¹¹, —OC₁-C₆-alkyl-OR¹¹, —SC₁-C₆-alkyl-C(O)OR¹¹, —C₂-C₆-alkenyl-C(═O)OR¹¹, —NR¹¹—C(═O)—C₁-C₆-alkyl-C(═O)OR¹¹, —NR¹¹—C(═O)—C₁-C₆-alkenyl-C(═O)OR¹¹, —C(O)OR¹¹, C(O)R¹¹, or —C₂-C₆-alkenyl-C(═O)R¹¹, ═O, or —C₂-C₆-alkenyl-C(═O)—NR¹¹R¹²,
C₁-C₆-alkyl, C₂-C₆-alkenyl or C₂-C₆-alkynyl, each of which may optionally be substituted with one or more substituents independently selected from R¹³,
aryl, aryloxy, aryloxycarbonyl, aroyl, arylsulfanyl, aryl-C₁-C₆-alkoxy, aryl-C₁-C₆-alkyl, aryl-C₂-C₆-alkenyl, aroyl-C₂-C₆-alkenyl, aryl-C₂-C₆-alkynyl, heteroaryl, heteroaryl-C₁-C₆-alkyl, heteroaryl-C₂-C₆-alkenyl, heteroaryl-C₂-C₆-alkynyl, or C₃-C₆ cycloalkyl,
of which each cyclic moiety may optionally be substituted with one or more substituents independently selected from R¹⁴,
R¹¹ and R¹² are independently selected from hydrogen, OH, C₁-C₂₀-alkyl, aryl-C₁-C₆-alkyl or aryl, wherein the alkyl groups may optionally be substituted with one or more substituents independently selected from R¹⁵, and the aryl groups may optionally be substituted one or more substituents independently selected from R¹⁶; R¹¹ and R¹² when attached to the same nitrogen atom may form a 3 to 8 membered heterocyclic ring with the said nitrogen atom, the heterocyclic ring optionally containing one or two further heteroatoms selected from nitrogen, oxygen and sulphur, and optionally containing one or two double bonds,
R¹³ independently selected from halogen, —CN, —CF₃, —OCF₃, —OR¹¹, —C(O)OR¹¹, —NR¹¹R¹², and —C(O)NR¹¹R¹²,
R¹⁴ is independently selected from halogen, —C(O)OR¹¹, —CH₂C(O)OR¹¹, —CH₂OR¹¹, —CN, —CF₃, —OCF₃, —NO₂, —OR¹¹, —NR¹¹R¹², —NR¹¹C(O)R¹¹, —S(O)₂R¹¹, aryl and C₁-C₆-alkyl,
R¹⁵ is independently selected from halogen, —CN, —CF₃, ═O, —OCF₃, —OC₁-C₆-alkyl, —C(O)OC₁-C₆-alkyl, —COOH and —NH₂,
R¹⁶ is independently selected from halogen, —C(O)OC₁-C₆-alkyl, —COOH, —CN, —CF₃, —OCF₃, —NO₂, —OH, —OC₁-C₆-alkyl, —NH₂, C(═O) or C₁-C₆-alkyl, or any enantiomer, diastereomer, including a racemic mixture, tautomer as well as a salt thereof with a pharmaceutically acceptable acid or base.

5. The pharmaceutical composition according to claim 4 wherein X is ═O or ═S.

6. The pharmaceutical composition according to claim 4 wherein Y is —O— or —S—.

7. The pharmaceutical composition according to claim 4 wherein A is aryl optionally substituted with up to four substituents, R⁷, R⁸, R⁹, and R¹⁰ which may be the same or different.

8. The pharmaceutical composition according to claim 7 wherein A is selected from ArG1 optionally substituted with up to four substituents, R⁷, R⁸, R⁹, and R¹⁰ which may be the same or different.

9. The pharmaceutical composition according to claim 8 wherein A is phenyl or naphtyl optionally substituted with up to four substituents, R⁷, R⁸, R⁹, and R¹⁰ which may be the same or different.

10. The pharmaceutical composition according to claim 9 wherein A is

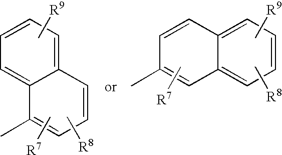

11. The pharmaceutical composition according to claim 4 wherein R¹ is hydrogen.

12. The pharmaceutical composition according to claim 4 wherein R² is hydrogen.

13. The pharmaceutical composition according to claim 4 wherein R¹ and R² are combined to form a double bond.

14. The pharmaceutical composition according to claim 4 wherein R³ is C₁-C₆-alkyl, halogen, or C(O)NR¹⁶R¹⁷.

15. The pharmaceutical composition according to claim 4 wherein B is phenyl optionally substituted with up to four substituents, R⁷, R⁸, R⁹, and R¹⁰ which may be the same or different.

16. The pharmaceutical composition according to claim 4 wherein R⁷, R⁸, R⁹ and R¹⁰ are independently selected from
hydrogen, halogen, —NO₂, —OR¹¹, —NR¹¹R¹², —SR¹¹, —NR¹¹S(O)₂R¹², —S(O)₂NR¹¹R¹², —S(O)NR¹¹R¹², —S(O)R¹¹, —S(O)₂R¹¹, —OS(O)₂R¹¹, —NR¹¹C(O)R¹², —CH₂OR¹¹, —CH₂OC—(O)R¹¹, —CH₂NR¹¹R¹², —OC(O)R¹¹, —OC₁-C₆-alkyl-C(O)OR¹¹, —OC₁-C₆-alkyl-C(O)—NR¹¹R¹², —OC₁-C₆-alkyl-OR¹¹, —SC₁-C₆-alkyl-C(O)OR¹¹, —C₂-C₆-alkenyl-C(═O)OR¹¹, —C(O)OR¹¹, or —C₂-C₆-alkenyl-C(═O)R¹¹,
C₁-C₆-alkyl, C₂-C₆-alkenyl or C₂-C₆-alkynyl, which may each optionally be substituted with one or more substituents independently selected from R¹³
aryl, aryloxy, aroyl, arylsulfanyl, aryl-C₁-C₆-alkoxy, aryl-C₁-C₆-alkyl, aryl-C₂-C₆-alkenyl, aroyl-C₂-C₆-alkenyl, aryl-C₂-C₆-alkynyl, heteroaryl, heteroaryl-C₁-C₆-alkyl, wherein each of the cyclic moieties optionally may be substituted with one or more substituents independently selected from R¹⁴.

17. The pharmaceutical composition according to claim 16 wherein R⁷, R⁸, R⁹ and R¹⁰ are independently selected from
hydrogen, halogen, —NO₂, —OR¹¹, —NR¹¹R¹², —SR¹¹, —S(O)₂R¹¹, —OS(O)₂R¹¹, —CH₂OC—(O)R¹¹, —OC(O)R¹¹, —OC₁-C₆-alkyl-C(O)OR¹¹, —OC₁-C₆-alkyl-OR¹¹, —SC₁-C₆-alkyl-C—(O)OR¹¹, —C(O)OR¹¹, or —C₂-C₆-alkenyl-C(═O)R¹¹,
C₁-C₆-alkyl or C₁-C₆— which may each optionally be substituted with one or more substituents independently selected from R¹³
aryl, aryloxy, aroyl, aryl-C₁-C₆-alkoxy, aryl-C₁-C₆-alkyl, heteroaryl,
of which each of the cyclic moieties optionally may be substituted with one or more substituents independently selected from R¹⁴.

18. The pharmaceutical composition according to claim 4 wherein R¹¹ and R¹² are independently selected from hydrogen, C₁-C₂₀-alkyl, aryl or aryl-C₁-C₆-alkyl, wherein the alkyl groups may optionally be substituted with one or more substituents independently selected from R¹⁵, and the aryl groups may optionally be substituted one or more substituents independently selected from R¹⁶; R¹¹ and R¹² when attached to the same nitrogen atom may form a 3 to 8 membered heterocyclic ring with the said nitrogen atom, the heterocyclic ring optionally containing one or two further heteroatoms selected from nitrogen, oxygen and sulphur, and optionally containing one or two double bonds.

19. The pharmaceutical composition according to claim 18 wherein R¹¹ and R¹² are independently selected from phenyl or phenyl-C₁-C₆-alkyl.

20. The pharmaceutical composition according to claim 4 wherein R¹³ is independently selected from halogen, CF₃, OR¹¹ or NR¹¹, R¹².

21. The pharmaceutical composition according to claim 4 wherein R¹⁴ is independently selected from halogen, —C(O)OR¹¹, —CN, —CF₃, —OR¹¹, S(O)₂R¹¹, and C₁-C₆alkyl.

22. The pharmaceutical composition according to claim 4 wherein R¹⁵ is independently selected from halogen, —CN, —CF₃, —C(O)OC₁-C₆-alkyl, and —COOH.

23. The pharmaceutical composition according to claim 4 wherein R¹⁶ is independently selected from halogen, —C(O)OC₁-C₆-alkyl, —COOH, —NO₂, —OC₁-C₆-alkyl, —NH₂, C(═O) or C₁-C₆-alkyl.

24. The pharmaceutical composition according to claim 1 wherein the ligand for the His^{B10} anion site is the SCN⁻ anion.

25. The pharmaceutical composition according to claim 1 wherein the ligand for the His^{B10} anion site is the Cl⁻ anion.

26. The pharmaceutical composition according to claim 1 wherein the insulin peptide is selected from the group consisting of human insulin, an analogue thereof, a derivative thereof, and combinations of any of these.

27. The pharmaceutical composition according to claim 26 wherein the insulin is an analogue of human insulin selected from the group consisting of
i. An analogue wherein position B28 is Asp, Lys, Leu, Val, or Ala and position B29 is Lys or Pro; and
ii. des(B28-B30), des(B27) or des(B30) human insulin.

28. The pharmaceutical composition according to claim 27, wherein the human insulin analogue is Asp^{B28}-human insulin.

29. The pharmaceutical composition according to claim 26, wherein the human insulin analogue is B29-N^{ε}-myristoyl-des(B30) human insulin.

30. The pharmaceutical composition according to claim 1 wherein the insulinotropic peptide is GLP-1(7-37), a GLP-1(7-37) analogue, a derivative of GLP-1(7-37), or a derivative of a GLP-1(7-37) analogue.

31. The pharmaceutical composition according to claim 30 wherein the GLP-1(7-37) analogue is selected from the group consisting of Arg³⁴-GLP-1(7-37), Gly⁸-GLP-1(7-36)-amide, Gly⁸-GLP-1(7-37), Val⁸-GLP-1(7-36)-amide, Val⁸-GLP-1(7-37), Val⁸Asp²²-GLP-1(7-36)-amide, Val⁸Asp²²-GLP-1(7-37), Val⁸Glu²²-GLP-1(7-36)-amide, Val⁸Glu²²-GLP-1(7-37), Val⁸Lys²²-GLP-1(7-36)-amide, Val⁸Lys²²-GLP-1(7-37), Val⁸Arg²²-GLP-1(7-36)-amide, Val⁸Arg²²-GLP-1(7-37), Val⁸His²²-GLP-1(7-36)-amide, Val⁸His²²-GLP-1(7-37), Val⁸Trp¹⁶Glu²²-GLP-1(7-37), Val⁸Glu²²Val²⁵-GLP-1(7-37), Val⁸Tyr¹⁶Glu²²-GLP-1(7-37), Val⁸Trp¹⁶Glu²²-GLP-1(7-37), Val⁸Leu¹⁶Glu²²-GLP-1(7-37), Val⁸Tyr¹⁸Glu²²-GLP-1(7-37), Val⁸Glu²²His³⁷-GLP-1(7-37), Val⁸Glu²²,Ile³³-GLP-1(7-37), Val⁸Trp¹⁶Glu²²Val²⁵Ile³³-GLP-1(7-37), Val⁸Trp¹⁶Glu²²Ile³³-GLP-1(7-37), Val⁸Glu²² Val²⁵Ile³³-GLP-1(7-37), Val⁸Trp¹⁶Glu²²Val²⁵-GLP-1(7-37), analogues thereof and derivatives of any of these.

32. The pharmaceutical composition according to claim 30 wherein the derivative of a GLP-1(7-37) analogue is GLP-1(7-36)-amide.

33. The pharmaceutical composition according to claim 30 wherein the derivative of a GLP-1(7-37) analogue is Arg³⁴, Lys²⁶(N^{ε}-(γ-Glu(N^{α}-hexadecanoyl)))GLP-1(7-37).

34. The pharmaceutical composition according to claim 1 wherein the insulinotropic peptide is exendin-4, an exendin-4 analogue, a derivative of exendin-4, or a derivative of an exendin-4 analogue.

35. The pharmaceutical composition according to claim 34 wherein the exendin-4 analogue is exendin-3 or ZP-10 (HGEGTFTSDLSKQMEEEAVRLFIEWLKNGGPSSGAPPSKKKKKK-NH2).

36. The pharmaceutical composition according to claim 1 wherein said pharmaceutical composition comprises 0-6 moles zinc²⁺ ions per mole insulin hexamer.

37. The pharmaceutical composition according to claim 36 comprising 2-3 moles zinc²⁺ ions per mole insulin hexamer.

38. The pharmaceutical composition according to claim 36 comprising 3-6 moles zinc²⁺ ions per mole insulin hexamer.

39. The pharmaceutical composition according to claim 1 wherein said pharmaceutical composition c have a pH value in the range of 6.5 to 9, e.g. 7 to 8.5, or in the range of 7.4 to 8.2.

40. The pharmaceutical composition according to claim 1 wherein the pharmaceutical composition comprises a surfactant.

41. The pharmaceutical composition according to claim 40 wherein the surfactant is selected from polysorbate and poloxamer.

42. The pharmaceutical composition according to claim 41 wherein the surfactant is polysorbate-20.

43. The pharmaceutical composition according to claim 41 wherein the surfactant is poloxamer 188.

44. The pharmaceutical composition according to claim 1 wherein the pharmaceutical composition comprises a preservative.

45. The pharmaceutical composition according to claim 44 wherein the preservative is phenol, m-cresol or a mixture thereof.

46. The pharmaceutical composition according to claim 1, wherein the pharmaceutical composition comprises a stabiliser.

47. The pharmaceutical composition according to claim 46, wherein the stabiliser is selected from the group consisting of L-glycine, L-histidine and arginine.

48. The pharmaceutical composition according to claim 46, wherein the stabiliser is a polyethylene glycol.

49. The pharmaceutical composition according to claim 1 wherein the pharmaceutical composition comprises an isotonicity agent.

50. The pharmaceutical composition according to claim 49 wherein the isotonicity agent is selected from glycerol, mannitol sorbitol, propylene glycol or a mixture thereof.

51. The pharmaceutical composition according to claim 1 wherein the pharmaceutical composition comprises a buffer substance.

52. The pharmaceutical composition according to claim 51 wherein the buffer substance is sodium phosphate, TRIS, HEPES, glycine and sodium citrate, or a mixture thereof.

53. The pharmaceutical composition according to claim 4 wherein the insulin peptide is selected from the group consisting of human insulin, B28 Asp human insulin, B28 Lys B29 Pro human insulin, B3 Lys B29 Glu human insulin or B29-N^{ε}-myristoyl-des(B30) human insulin, and the insulinotropic peptide is selected from Arg³⁴ Lys²⁶(N^{ε}-(γ-Glu(N^{α}-hexadecanoyl)))-GLP-1(7-37), HGEGTFTSDLSKQMEEEAVRLFIEWLKNGGPSSGAPPSKKKKKK-NH2 or exendin-4.

54. The pharmaceutical composition according to claim 1 wherein the insulin peptide is selected from the group consisting of human insulin, B28 Asp human insulin, B28 Lys B29 Pro human insulin, B3 Lys B29 Glu human insulin or B29-N^{ε}-myristoyl-des(B30) human insulin, and the insulinotropic peptide is selected from Arg³⁴ Lys²⁶(N^{ε}-(γ-Glu(N^{α}-hexadecanoyl)))-GLP-1(7-37), HGEGTFTSDLSKQMEEEAVRLFIEWLKNGGPSSGAPPSKKKKKK-NH2 or exendin-4, and the ligand for the His^{B10} anion site is a SCN⁻ anion.

55. The pharmaceutical composition according to claim 1 wherein the insulin peptide is selected from the group consisting of human insulin, B28 Asp human insulin, B28 Lys B29 Pro human insulin, B3 Lys B29 Glu human insulin or B29-N^{ε}-myristoyl-des(B30) human insulin, and the insulinotropic peptide is selected from Arg³⁴ Lys²⁶(N^{ε}-(γ-Glu(N^{α}-hexadecanoyl)))-GLP-1(7-37), HGEGTFTSDLSKQMEEEAVRLFIEWLKNGGPSSGAPPSKKKKKK-NH2 or exendin-4, and the ligand for the His^{B10} anion site is a Cl⁻ anion.

56. The pharmaceutical composition according to claim 4 wherein the insulin peptide is selected from the group consisting of human insulin, B28 Asp human insulin, B28 Lys B29 Pro human insulin, B3 Lys B29 Glu human insulin or B29-N^{ε}-myristoyl-des(B30) human insulin, and the insulinotropic peptide is selected from Arg³⁴ Lys²⁶(N^{ε}-(γ-Glu(N^{α}-hexadecanoyl)))-GLP-1(7-37), HGEGTFTSDLSKQMEEEAVRLFIEWLKNGGPSSGAPPSKKKKKK-NH2 or exendin-4, and a surfactant selected from a polysorbate or poloxamer.

57. The pharmaceutical composition according to claim 1 wherein the insulin peptide is selected from the group consisting of human insulin, B28 Asp human insulin, B28 Lys B29 Pro human insulin, B3 Lys B29 Glu human insulin or B29-N^{ε}-myristoyl-des(B30) human insulin, and the insulinotropic peptide is selected from Arg³⁴ Lys²⁶(N^{ε}-(γ-Glu(N^{α}-hexadecanoyl)))-GLP-1(7-37), HGEGTFTSDLSKQMEEEAVRLFEWLKNGGPSSGAPPSKKKKKK-NH2 or exendin-4, and the ligand for the His^{B10} anion site is a SCN⁻ anion, and a surfactant selected from a polysorbate or poloxamer.

58. The pharmaceutical composition according to claim 1 wherein the insulin peptide is selected from the group consisting of human insulin, B28 Asp human insulin, B28 Lys B29 Pro human insulin, B3 Lys B29 Glu human insulin or B29-N^{ε}-myristoyl-des(B30) human insulin, and the insulinotropic peptide is selected from Arg³⁴ Lys²⁶(N^{ε}-(γ-Glu(N^{α}-hexadecanoyl)))-GLP-1(7-37), HGEGTFTSDLSKQMEEEAVRLFIEWLKNGGPSSGAPPSKKKKKK-NH2 or exendin-4, and the ligand for the His^{B10} anion site is a Cl⁻ anion, and a surfactant is selected from a polysorbate or poloxamer.

59. A method for treating hyperglycemia comprising administering to a patient in need thereof a pharmaceutically effective amount of a pharmaceutical composition according to claim 1.

60. (canceled)
